(19) United States
(12) Patent Application Publication
(10) Pub. No.: US 2025/0214996 A1
(43) Pub. Date: Jul. 3, 2025

(54) SALTS AND SOLID FORMS OF AN ESTROGEN RECEPTOR ANTAGONIST

(71) Applicant: Olema Pharmaceuticals, Inc., San Francisco, CA (US)

(72) Inventors: David C. Myles, Berkeley, CA (US); Rampurna Prasad Gullapalli, San Diego, CA (US); Jing Jim Zhang, San Francisco, CA (US); David Askin, Warren, NJ (US); Thomas San le, Evanston, IL (US); Mitulkumar Patel, Robbinsville, NJ (US); Ekaterina Albert, West Lafayette, IN (US); Liping Wang, Tianjin (CN); Lina Yang, Tianjin (CN); Shuyan Huang, Tianjin (CN)

(21) Appl. No.: 18/867,291

(22) PCT Filed: May 19, 2023
(86) PCT No.: PCT/US23/22983

§ 371 Date: Nov. 19, 2024

(30) Foreign Application Priority Data
May 20, 2022 (WO) PCT/CN2022/094230

(51) Int. Cl. C07D 471/04; A61K 31/437; A61K 45/06

(52) U.S. Cl. CPC C07D 471/04; A61K 31/437; A61K 45/06

(57) ABSTRACT

The present disclosure provides solid and salt forms of an estrogen receptor (ER) inhibitor, compositions thereof and N methods of treating a ER-mediated disorder.

### RELATED APPLICATIONS

This application claims priority to and benefit of PCT Application No. PCT/CN2022/094230, filed May 20, 2022, the entire contents of which is hereby incorporated by reference.

### BACKGROUND

The estrogen receptor (ER) plays important roles in various cancers, including breast cancers. A variety of treatments have been developed to target the estrogen receptor and/or its activities.

### SUMMARY

There remains a need for anti-estrogen agents that can completely inhibit estrogen receptors, including those coded for by both wild-type and mutant versions (e.g., those containing activating mutations) of the gene encoding Estrogen Receptor-alpha (ERα), Estrogen Receptor 1 (ESR1). Selective estrogen receptor modulators (SERMs) or degraders (SERDs) are a particularly useful or promising tools for such therapy. Recently, classes of estrogen receptor antagonists, termed Complete Estrogen Receptor Antagonists (CERANs) have emerged as promising therapies for completely inhibiting the estrogen receptor.

CERANs are considered “complete” as compared to other estrogen receptor antagonists because they inactivate two distinct transcriptional activation functions (AF1 and AF2) of the estrogen receptor. Previous therapies that are not CERANs fail when activation mutations in the gene that codes for estrogen receptor 1 allows for activation of both AF1 and AF2 even in the absence of estrogen. The present disclosure provides salts, solid forms, and compositions and uses thereof of a compound useful for complete antagonism of the estrogen receptor, providing an option for treatment for subjects suffering from a cancer, and/or wherein the subject carries a mutation of estrogen receptor 1 (ESR1).

The compound (1R,3R)-2-(2-fluoro-2-methylpropyl)-3-methyl-1-(4-((1-propylazetidin-3-yl)oxy)phenyl)-2,3,4,9-tetrahydro-1H-pyrido[3,4-b]indole (“Compound 1”):

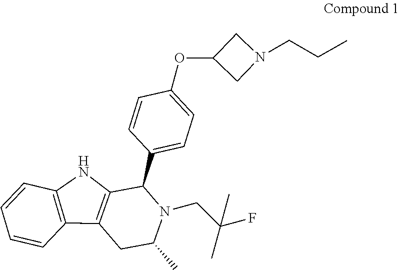 [SMILES: CCCN1CC(Oc2ccc([C@@H]3c4[nH]c5ccccc5c4C[C@@H](C)N3CC(C)(C)F)cc2)C1]

is a complete estrogen receptor antagonist published in PCT Publication No. WO 2017/059139 (the entire contents of which are hereby incorporated by reference), designated as Compound B. There remains a need for identifying salt, solid, hydrate, and/or solvate forms of Compound 1 useful for various therapeutic applications.

In some embodiments, the present disclosure provides one or more solid forms of Compound 1.

In some embodiments, the present disclosure provides a solid form of Compound 1, wherein Compound 1 is a free base.

In some embodiments, the present disclosure provides one or more solvates of Compound 1, (e.g., a complex of Compound 1 and a solvent, including, for example, acetonitrile, acetone, dimethylsulfoxide, tetrahydrofuran, dioxane, N-methylpyrrolidone, and ethyl acetate solvates).

In some embodiments, the present disclosure provides one or more crystalline forms of Compound 1.

In some embodiments, the present disclosure provides salt forms of Compound 1, designated as Compound 2:

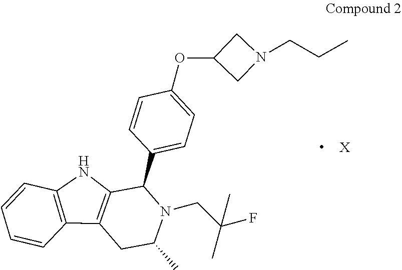

wherein X is a co-former selected from the group consisting of maleic acid, fumaric acid, oxalic acid, and phosphoric acid.

In some embodiments, the present disclosure provides one or more solvates of Compound 2 (e.g., a complex of Compound 2 and a solvent, including, for example, acetonitrile, ethyl acetate, methyl isobutyl ketone, and tert-butyl acetate solvates). In some embodiments, the present disclosure provides one or more hydrates of Compound 2. In some embodiments, the present disclosure provides one or more unsolvated forms of Compound 2.

In some embodiments, the present disclosure provides one or more crystalline forms of Compound 2.

In some embodiments, the present disclosure provides methods of inhibiting the estrogen receptor, or a mutation thereof, in a biological sample comprising contacting said biological sample with an estrogen receptor antagonist (e.g., a form of Compound 1 or Compound 2 provided herein).

In some embodiments, the present disclosure provides compositions comprising one or more forms of Compound 1 or Compound 2 provided herein. In some embodiments, the present disclosure provides pharmaceutical compositions comprising one or more forms of Compound 1 or Compound 2 provided herein and a pharmaceutically acceptable carrier.

In some embodiments, the present disclosure provides methods of treating patients or subjects suffering from a cancer related to the estrogen receptor or mutations of the estrogen receptor, comprising administering an estrogen receptor antagonist (e.g., a form of Compound 1 or Compound 2 provided herein).

In some embodiments, the present disclosure provides methods of treating estrogen receptor (ER)-associated diseases, disorders, and conditions (e.g., cancer) and/or for otherwise modulating (e.g., inhibiting) the estrogen receptor in the brain, comprising administering an estrogen receptor antagonist (e.g., a form of Compound 1 or Compound 2 provided herein).

In some embodiments, the present disclosure provides methods of treating an ER-associated disease disorder or condition (e.g., an ER-associated cancer, including but not limited to one that is or comprises tumor(s) in the brain such as brain metastases) by administering a particular complete estrogen receptor antagonist (e.g., a form of Compound 1 or Compound 2 provided herein) according to a regimen that achieves preferential accumulation in tumor relative to plasma in the patient (i.e., achieves accumulation in tumor to a concentration above that in plasma).

### BRIEF DESCRIPTION OF THE DRAWINGS

FIG. 1 is a series of X-ray powder diffraction (XRPD) patterns for Compound 1 Form A solvates (Acetonitrile Solvate, Acetone Solvate, and Tetrahydrofuran Solvate).

FIG. 2 is a thermogravimetric analysis (TGA) curve (top) and a differential scanning calorimetry (DSC) curve (bottom) of Compound 1 Form A Acetonitrile Solvate.

FIG. 3 is a series of XRPD patterns from samples of Compound 1 Form A Acetonitrile Solvate before and after heating at 70° C. and 80° C.

FIG. 4 is a dynamic vapor sorption (DVS) plot of Compound 1 Form A Acetonitrile Solvate.

FIG. 5 is series of XRPD patterns from samples of Compound 1 Form A Acetonitrile Solvate before and after DVS.

FIG. 6 is a XRPD pattern of Compound 1 Form A Acetonitrile Solvate.

FIG. 7 is additional TGA (top) and DSC (bottom) curves of Compound 1 Form A Acetonitrile Solvate.

FIG. 8 is an Oak Ridge Thermal Ellipsoid Plot (ORTEP) diagram of an asymmetric unit of a Compound 1 Form A Acetonitrile Solvate crystal, generated from single crystal X-ray crystallography, displaying thermal ellipsoids at 50% confidence interval.

FIG. 9 is a TGA curve (top) and a DSC curve (bottom) of Compound 1 Form A Acetone Solvate.

FIG. 10 is an ORTEP diagram of an asymmetric unit of a Compound 1 Form A Acetone Solvate crystal, generated from single crystal X-ray crystallography, displaying thermal ellipsoids at 50% confidence interval.

FIG. 11 is a TGA (top) and DSC (bottom) curves of Compound 1 Form A Tetrahydrofuran Solvate.

FIG. 12 is a series of XRPD patterns for Compound 1 Form A solvates (in order from top: Acetonitrile Solvate, Dioxane Solvate, N-Methylpyrrolidone (NMP) Solvate, and Ethyl Acetate Solvate), as well as the Kapton film used for specimen preparation (bottom).

FIG. 13 is an XRPD pattern of Compound 1 Form A Dioxane Solvate.

FIG. 14 is a TGA curve (top) and a DSC curve (bottom) of Compound 1 Form A Dioxane Solvate.

FIG. 15 is an XRPD pattern of Compound 1 Form A Ethyl Acetate Solvate.

FIG. 16 is a TGA curve (top) and a DSC curve (bottom) of Compound 1 Form A N-Methylpyrrolidone Solvate.

FIG. 17 is an XRPD pattern of Compound 1 Form B.

FIG. 18 is a TGA curve (top) and a DSC curve (bottom) of Compound 1 Form B.

FIG. 19 is a series of XRPD patterns from samples of Compound 1 Form B before and after heating at 215° C.

FIG. 20 is an ORTEP diagram of an asymmetric unit of a Compound 1 Form B crystal, generated from single crystal X-ray crystallography, displaying thermal ellipsoids at 50% confidence interval.

FIG. 21 is an XRPD pattern of Compound 1 Malate Form A.

FIG. 22 is a TGA curve (top) and a DSC curve (bottom) of Compound 1 Malate Form A.

FIG. 23 is a ¹H NMR spectrum of Compound 1 Malate Form A in DMSO-d₆.

FIG. 24 is an XRPD pattern of Compound 1 Fumarate Form A Anhydrate.

FIG. 25 is a TGA curve (top) and a DSC curve (bottom) of Compound 1 Fumarate Form A Anhydrate.

FIG. 26 is a ¹H NMR spectrum of Compound 1 Fumarate Form A Anhydrate in DMSO-d₆.

FIG. 27 is an XRPD pattern of Compound 1 Fumarate Form A Ethyl Acetate Solvate.

FIG. 28 is an XRPD pattern of Compound 1 Fumarate Form A Ethyl Acetate Solvate with indexing results.

FIG. 29 is a TGA curve (top) and a DSC curve (bottom) of Compound 1 Fumarate Form A Ethyl Acetate Solvate.

FIG. 30 is an ORTEP diagram of an asymmetric unit of a Compound 1 Fumarate Form A Ethyl Acetate Solvate crystal, generated from single crystal X-ray crystallography, displaying thermal ellipsoids at 50% confidence interval.

FIG. 31 is an XRPD pattern of Compound 1 Fumarate Form C.

FIG. 32 is a series of XRPD patterns for Compound 1 Fumarates (Form D, Form F, Form G, and Form A Ethyl Acetate Solvate, from top).

FIG. 33 is an XRPD pattern of Compound 1 Fumarate Form D.

FIG. 34 is a TGA curve (top) and a DSC curve (bottom) of Compound 1 Fumarate Form D.

FIG. 35A is an XRPD pattern of Compound 1 Fumarate Form E.

FIG. 35B is a TGA curve (top) and a DSC curve (bottom) of Compound 1 Fumarate Form E.

FIG. 36A is an XRPD pattern of Compound 1 Fumarate Form E.

FIG. 36B is a TGA curve of Compound 1 Fumarate Form E.

FIG. 36C is a DSC curve of Compound 1 Fumarate Form E.

FIG. 36D is a DVS plot of Compound 1 Fumarate Form E.

FIG. 37 is an XRPD pattern of Compound 1 Fumarate Form F.

FIG. 38 is a TGA curve (top) and a DSC curve (bottom) of Compound 1 Fumarate Form F.

FIG. 39 is an XRPD pattern of Compound 1 Fumarate Form G.

FIG. 40 is a TGA curve (top) and a DSC curve (bottom) of Compound 1 Fumarate Form G.

FIG. 41 is a series of XRPD patterns for Compound 1 Fumarate Form C (prepared by various methods) and Compound 1 Fumarate Form J, as well as the Kapton film used for specimen preparation.

FIG. 42 is an additional XRPD pattern of Compound 1 Fumarate Form A Anhydrate.

FIG. 43 is a TGA curve (top) and a DSC curve (bottom) of Compound 1 Fumarate Form A Anhydrate.

FIG. 44 is a series of XRPD patterns from samples of Compound 1 Fumarate Form A Anhydrate before and after slurrying in isopropanol for 1 day.

FIG. 45 is an XRPD pattern of Compound 1 Oxalate Form A.

FIG. 46 is a TGA curve (top) and a DSC curve (bottom) of Compound 1 Oxalate Form A.

FIG. 47 is an XRPD pattern of Compound 1 Phosphate Form A.

FIG. 48 is a TGA curve (top) and a DSC curve (bottom) of Compound 1 Phosphate Form A.

FIG. 49 is an XRPD pattern of Compound 1 Phosphate Form B.

FIG. 50 is a TGA curve (top) and a DSC curve (bottom) of Compound 1 Phosphate Form B.

FIG. 51 is a DVS plot of Compound 1 Malate Form A.

FIG. 52 is a series of XRPD patterns from samples of Compound 1 Malate Form A before and after DVS.

FIG. 53 is a DVS plot of Compound 1 Fumarate Form A Anhydrate.

FIG. 54 is a series of XRPD patterns from samples of Compound 1 Fumarate Form A Anhydrate before and after DVS.

FIG. 55 is a DVS plot of Compound 1 Oxalate Form A.

FIG. 56 is a series of XRPD patterns from samples of Compound 1 Oxalate Form A before and after DVS.

FIG. 57 is a series of XRPD patterns from competitive slurry experiments of Compound 1 Fumarate Form E and Compound 1 Fumarate Form A Anhydrate in isopropanol.

FIG. 58 is a series of XRPD patterns from competitive slurry experiments of Compound 1 Fumarate Form E and Compound 1 Fumarate Form A Anhydrate in water.

FIG. 59 is a series of XRPD patterns from competitive slurry experiments of Compound 1 Fumarate Form E and Compound 1 Fumarate Form A Anhydrate in ethyl acetate.

### DETAILED DESCRIPTION

### Compound 1

Compound 1 is a complete estrogen receptor antagonist, published in PCT Publication No. WO 2017/059139 (the entirety of which is incorporated herein by reference), designated as Compound B. Exemplary methods for using Compound 1 are described in PCT Publication Nos. WO 2021/007146 and WO 2021/178846, the entirety of each of which is incorporated herein by reference.

A synthesis of Compound 1 is described in detail in Example 10 of WO 2017/059139, as well as in Example 1 herein.

The present disclosure encompasses the recognition that it is desirable to provide a form (e.g., a salt and/or solid form) of Compound 1 that, as compared to another form of Compound 1 (e.g., an amorphous form), imparts characteristics such as improved stability, hygroscopicity, flow properties, compressibility, ease of processing, consistency in manufacturing, particle size distribution, bulk density, pharmacokinetics, bioavailability, and ease of formulation. For example, Applicant recognized that, when developing a solid dosage form (e.g., tablet or capsule) comprising Compound 1, the amorphous form of Compound 1 exhibited certain properties, e.g., flow properties, bulk density, and handleability, which made the process for generating a solid dosage form comprising Compound 1 difficult. Accordingly, the present disclosure provides salts and solid forms of Compound 1 which overcome the problems identified above.

### Solid Forms of Compound 1

In some embodiments, the present disclosure provides a solid form of Compound 1. In some embodiments, the present disclosure provides one or more polymorphic solid forms of Compound 1. As used herein, the term “polymorph” refers to the ability of a compound to exist in one or more different crystal structures. For example, one or more polymorphs may vary in pharmaceutically relevant physical properties between one form and another, e.g., solubility, stability, and/or hygroscopicity.

It will be appreciated that a solid form can exist in a neat or unsolvated form, a hydrated form, a solvated form, and/or a heterosolvated form. In some embodiments, a solid form of Compound 1 is a crystalline solid form of Compound 1. In some embodiments, a crystalline solid form of Compound 1 does not have any water or solvent incorporated into the crystalline structure (i.e., is “unsolvated”). In some embodiments, a crystalline solid form of Compound 1 does not have any water incorporated into the crystalline structure (i.e., is an “anhydrate”). In some embodiments, a crystalline solid form of Compound 1 is both unsolvated and an anhydrate.

In some embodiments, a crystalline solid form of Compound 1 comprises one or more equivalents of water and/or solvent (i.e., are hydrates and/or solvates, respectively). As used herein, the term “solvate” refers to a solid form with a stoichiometric or non-stoichiometric amount of one or more solvents incorporated into the crystal structure. For example, a solvated or heterosolvated polymorph can comprise 0.05, 0.1, 0.2, 0.5, 1.0, 1.5, 2.0, etc. equivalents independently of one or more solvents incorporated into the crystal lattice. As used herein, the term “hydrate” refers to a solvate, wherein the solvent incorporated into the crystal structure is water.

In some embodiments, the present disclosure provides Compound 1 as an acetone, dimethyl sulfoxide, tetrahydrofuran, dioxane, N-methylpyrrolidone, or ethyl acetate solvate.

It will be appreciated that certain solvates and/or hydrates of Compound 1 may be isostructural, i.e., are forms with essentially the same crystal lattice structure and differing only in the identity of the solvent within the crystal lattice. For example, as described herein, Compound 1 Form A may exist in a variety of different isostructural solvate forms (e.g., Acetonitrile Solvate, Acetone Solvate, Tetrahydrofuran Solvate, Dioxane Solvate, Ethyl Acetate Solvate, and N-Methylpyrrolidone Solvate).

As provided herein, crystalline solid forms of Compound 1 have distinct XRPD peaks that are not reported in previous disclosures of Compound 1. Provided herein are crystalline solid forms of Compound 1, compositions thereof, and methods of using and preparing crystalline solid forms of Compound 1.

As used herein, the term “about” when used in reference to a degree 2-theta value refers to the stated value±0.2 degrees 2-theta.

In some embodiments, provided forms (e.g., forms of Compound 1 and Compound 2) are characterized by having peaks in its XRPD pattern selected from “substantially all” of a provided list, optionally within ±0.2 degrees 2-theta of the stated value. It will be appreciated that an XRPD pattern having “substantially all” of a provided list of peaks refers to an XRPD pattern that comprises at least 80% (e.g., 80%, 85%, 90%, 95%, 99% or 100%) of the listed peaks. In some embodiments, an XRPD pattern comprises at least 90% of the listed peaks. In some embodiments, an XRPD pattern comprises all of the listed peaks. In some embodiments, an XRPD pattern comprises all but one of the listed peaks. In some embodiments, an XRPD pattern comprises all but two of the listed peaks. In some embodiments, an XRPD pattern comprises all but three of the listed peaks.

In some embodiments, provided forms (e.g., forms of Compound 1 and Compound 2) are characterized by having a pattern or spectrum that is “substantially similar” to a Figure provided herein. It will be appreciated that a pattern or spectrum having “substantial similarity” to a Figure provided herein is one that comprises one or more features (e.g., position (degrees 2-theta) values, temperature values, % weight loss values, intensity, shape of curve, etc.) of the provided Figure so as to enable identification of the form (e.g., solid and/or salt form) characterized by the pattern or spectrum as being the same as the form characterized in the Figure. For example, in some embodiments, an XRPD pattern having substantial similarity to a provided Figure is one that comprises substantially all of the same peaks, optionally within ±0.2 degrees 2-theta of peaks in the reference Figure. In some embodiments, an XRPD pattern having substantial similarity to a provided Figure is one that comprises substantially all of the same peaks, optionally within ±0.2 degrees 2-theta of peaks in the reference Figure, with about the same intensities.

### Compound 1 Form A

In some embodiments, the present disclosure provides Compound 1 as Form A. In some embodiments, Compound 1 Form A is a solvate of acetonitrile, acetone, tetrahydrofuran, dioxane, ethyl acetate, or N-methylpyrrolidone.

### Compound 1 Form A Acetonitrile Solvate

In some embodiments, the present disclosure provides Compound 1 as Form A Acetonitrile Solvate.

In some embodiments, Compound 1 Form A Acetonitrile Solvate is characterized by one or more peaks in its XRPD pattern selected from those at about 10.34, about 11.49, about 17.66, about 19.00, and about 19.76 degrees 2-theta. In some embodiments, Compound 1 Form A Acetonitrile Solvate is characterized by two or more peaks in its XRPD pattern selected from those at about 10.34, about 11.49, about 17.66, about 19.00, and about 19.76 degrees 2-theta. In some embodiments, Compound 1 Form A Acetonitrile Solvate is characterized by three or more peaks in its XRPD pattern selected from those at about 10.34, about 11.49, about 17.66, about 19.00, and about 19.76 degrees 2-theta.

In some embodiments, Compound 1 Form A Acetonitrile Solvate is characterized by peaks in its XRPD pattern at about 10.34, about 11.49, about 17.66, about 19.00, and about 19.76 degrees 2-theta. In some embodiments, Compound 1 Form A Acetonitrile Solvate is characterized by peaks in its XRPD pattern at substantially all of:

Position ± 0.2 (degrees 2-Theta)

5.31
10.34
10.61
11.49
12.40
13.50
14.72
16.04
17.45
17.66
18.02
18.43
19.00
19.76
20.44
20.66
21.60
22.03
23.03
23.87
25.07
25.27
26.37
27.10
27.65
29.13
30.63

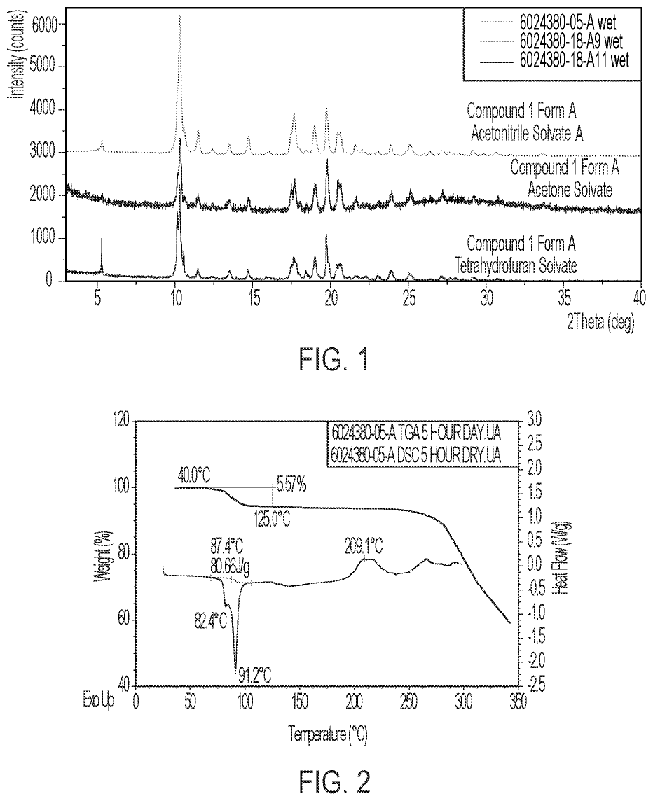

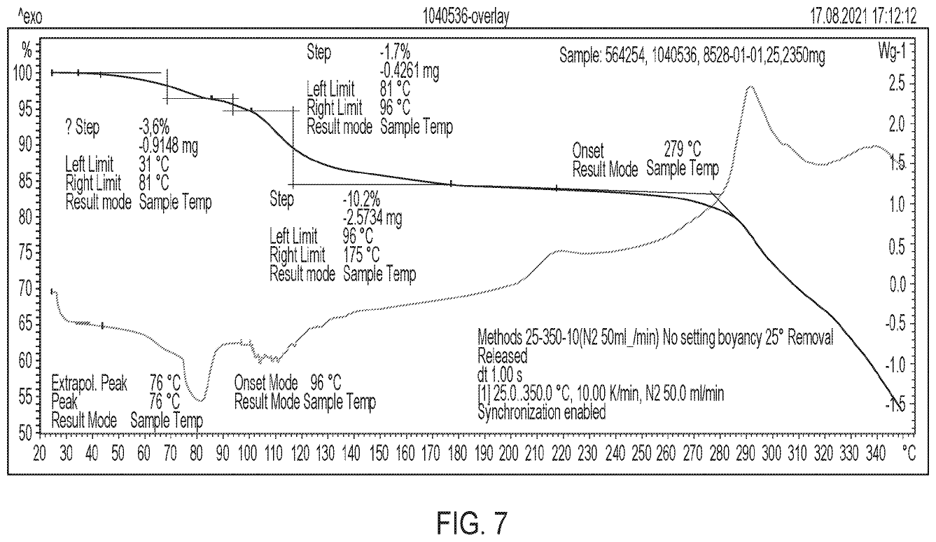

In some embodiments, Compound 1 Form A Acetonitrile Solvate is characterized by one or more of the following:
(i) an XRPD pattern substantially similar to that depicted in FIG. 1 (top) and/or FIG. 6;
(ii) a TGA pattern substantially similar to that depicted in FIG. 2 and/or FIG. 7; and
(iii) a DSC pattern substantially similar to that depicted in FIG. 2 and/or FIG. 7.

### Compound 1 Form A Acetone Solvate

In some embodiments, the present disclosure provides Compound 1 as Form A Acetone Solvate.

In some embodiments, Compound 1 Form A Acetone Solvate is characterized by one or more peaks in its XRPD pattern selected from those at about 10.33, about 17.69, about 19.05, about 19.80, and about 20.49 degrees 2-theta. In some embodiments, Compound 1 Form A Acetone Solvate is characterized by two or more peaks in its XRPD pattern selected from those at about 10.33, about 17.69, about 19.05, about 19.80, and about 20.49 degrees 2-theta. In some embodiments, Compound 1 Form A Acetone Solvate is characterized by three or more peaks in its XRPD pattern selected from those at about 10.33, about 17.69, about 19.05, about 19.80, and about 20.49 degrees 2-theta.

In some embodiments, Compound 1 Form A Acetone Solvate is characterized by peaks in its XRPD pattern at about 10.33, about 17.69, about 19.05, about 19.80, and about 20.49 degrees 2-theta. In some embodiments, Compound 1 Form A Acetone Solvate is characterized by peaks in its XRPD pattern at substantially all of:

Position ± 0.2 (degrees 2-Theta)

4.72
5.31
10.33
11.51
13.54
14.72
17.47
17.69
18.47
19.05
19.80
20.49
21.69
23.94
25.13
27.22
29.21
33.71

In some embodiments, Compound 1 Form A Acetone Solvate is characterized by one or more of the following:
(i) an XRPD pattern substantially similar to that depicted in FIG. 1 (middle);
(ii) a TGA pattern substantially similar to that depicted in FIG. 9; and
(iii) a DSC pattern substantially similar to that depicted in FIG. 9.

### Compound 1 Form A Tetrahydrofuran Solvate

In some embodiments, the present disclosure provides Compound 1 as Form A Tetrahydrofuran Solvate.

In some embodiments, Compound 1 Form A Tetrahydrofuran Solvate is characterized by one or more peaks in its XRPD pattern selected from those at about 5.29, about 10.16, about 10.31, about 10.57, and about 19.75 degrees 2-theta. In some embodiments, Compound 1 Form A Tetrahydrofuran Solvate is characterized by two or more peaks in its XRPD pattern selected from those at about 5.29, about 10.16, about 10.31, about 10.57, and about 19.75 degrees 2-theta degrees 2-theta. In some embodiments, Compound 1 Form A Tetrahydrofuran Solvate is characterized by three or more peaks in its XRPD pattern selected from those at about 5.29, about 10.16, about 10.31, about 10.57, and about 19.75 degrees 2-theta.

In some embodiments, Compound 1 Form A Tetrahydrofuran Solvate is characterized by peaks in its XRPD pattern at about 5.29, about 10.16, about 10.31, about 10.57, and about 19.75 degrees 2-theta. In some embodiments, Compound 1 Form A Tetrahydrofuran Solvate is characterized by peaks in its XRPD pattern at substantially all of:

Position ± 0.2 (degrees 2-Theta)

5.29
10.16
10.31
10.57
11.47
13.52
14.69
15.86
17.81
18.42
18.99
19.75
20.40
20.73
21.57
22.31
23.04
23.83
23.95
25.04
27.12
27.72
29.12

In some embodiments, Compound 1 Form A Tetrahydrofuran Solvate is characterized by one or more of the following:
(i) an XRPD pattern substantially similar to that depicted in FIG. 1 (bottom);
(ii) a TGA pattern substantially similar to that depicted in FIG. 11; and
(iii) a DSC pattern substantially similar to that depicted in FIG. 11.

### Compound 1 Form A Dioxane Solvate

In some embodiments, the present disclosure provides Compound 1 as Form A Dioxane Solvate.

In some embodiments, Compound 1 Form A Dioxane Solvate is characterized by one or more peaks in its XRPD pattern selected from those at about 10.10, about 10.25, about 17.54, about 18.82, about 19.64, and about 20.34 degrees 2-theta. In some embodiments, Compound 1 Form A Dioxane Solvate is characterized by two or more peaks in its XRPD pattern selected from those at about 10.10, about 10.25, about 17.54, about 18.82, about 19.64, and about 20.34 degrees 2-theta. In some embodiments, Compound 1 Form A Dioxane Solvate is characterized by three or more peaks in its XRPD pattern selected from those at about 10.10, about 10.25, about 17.54, about 18.82, about 19.64, and about 20.34 degrees 2-theta.

In some embodiments, Compound 1 Form A Dioxane Solvate is characterized by peaks in its XRPD pattern at about 10.10, about 10.25, about 17.54, about 18.82, about 19.64, and about 20.34 degrees 2-theta. In some embodiments, Compound 1 Form A Dioxane Solvate is characterized by peaks in its XRPD pattern at substantially all of:

Position ± 0.2 (degrees 2-Theta)

5.25
10.10
10.25
10.53
11.40
13.37
14.61
17.35
17.54
18.82
19.64
20.34
21.48
21.90

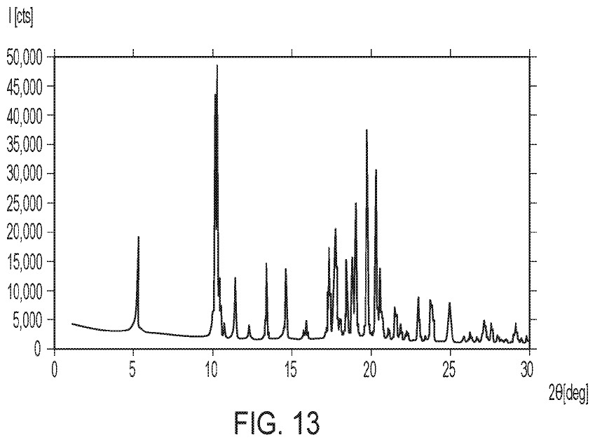

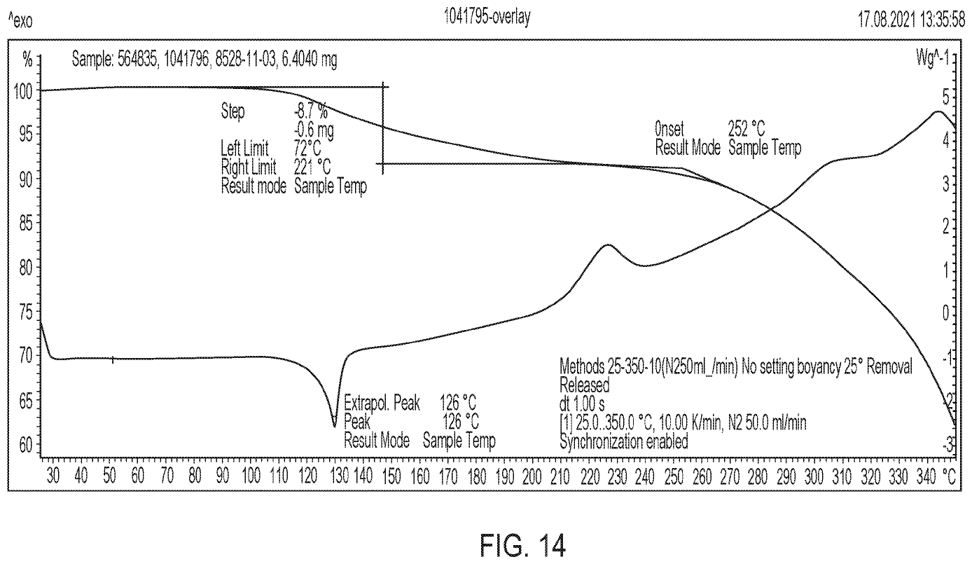

In some embodiments, Compound 1 Form A Dioxane Solvate is characterized by one or more of the following:
(i) an XRPD pattern substantially similar to that depicted in FIG. 13;
(ii) a TGA pattern substantially similar to that depicted in FIG. 14; and
(iii) a DSC pattern substantially similar to that depicted in FIG. 14.

### Compound 1 Form A Ethyl Acetate Solvate

In some embodiments, the present disclosure provides Compound 1 as Form A Ethyl Acetate Solvate.

In some embodiments, Compound 1 Form A Ethyl Acetate Solvate is characterized by one or more peaks in its XRPD pattern selected from those at about 10.10, about 10.25, about 17.54, about 18.82, about 19.64, and about 20.34 degrees 2-theta. In some embodiments, Compound 1 Form A Ethyl Acetate Solvate is characterized by two or more peaks in its XRPD pattern selected from those at about 10.10, about 10.25, about 17.54, about 18.82, about 19.64, and about 20.34 degrees 2-theta. In some embodiments, Compound 1 Form A Ethyl Acetate Solvate is characterized by three or more peaks in its XRPD pattern selected from those at about 10.10, about 10.25, about 17.54, about 18.82, about 19.64, and about 20.34 degrees 2-theta.

In some embodiments, Compound 1 Form A Ethyl Acetate Solvate is characterized by peaks in its XRPD pattern at about 10.10, about 10.25, about 17.54, about 18.82, about 19.64, and about 20.34 degrees 2-theta. In some embodiments, Compound 1 Form A Ethyl Acetate Solvate is characterized by peaks in its XRPD pattern at substantially all of:

Position ± 0.2 (degrees 2-Theta)

5.25
10.10
10.25
10.53
11.40
13.37
14.61
17.35
17.54
18.82
19.64
20.34
21.48
21.90

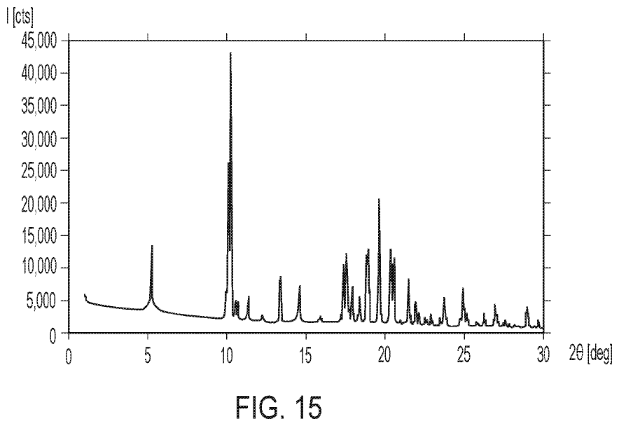

In some embodiments, Compound 1 Form A Ethyl Acetate Solvate is characterized by an XRPD pattern substantially similar to that depicted in FIG. 15.

### Compound 1 Form A N-Methylpyrrolidone Solvate

In some embodiments, the present disclosure provides Compound 1 as Form A N-Methylpyrrolidone Solvate.

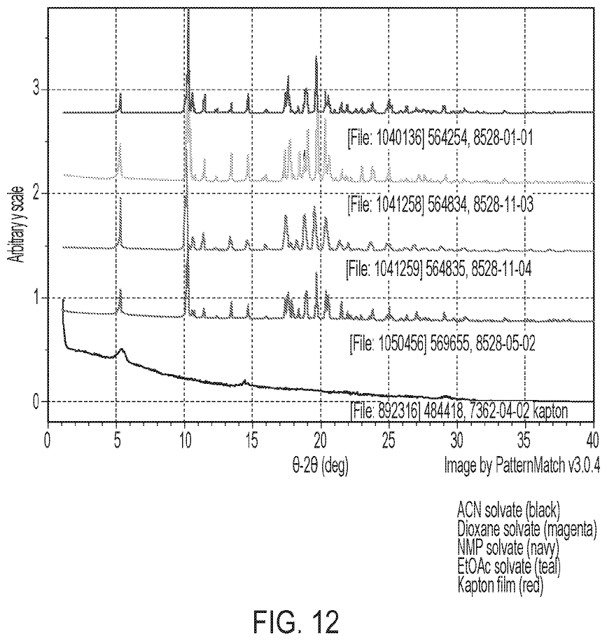

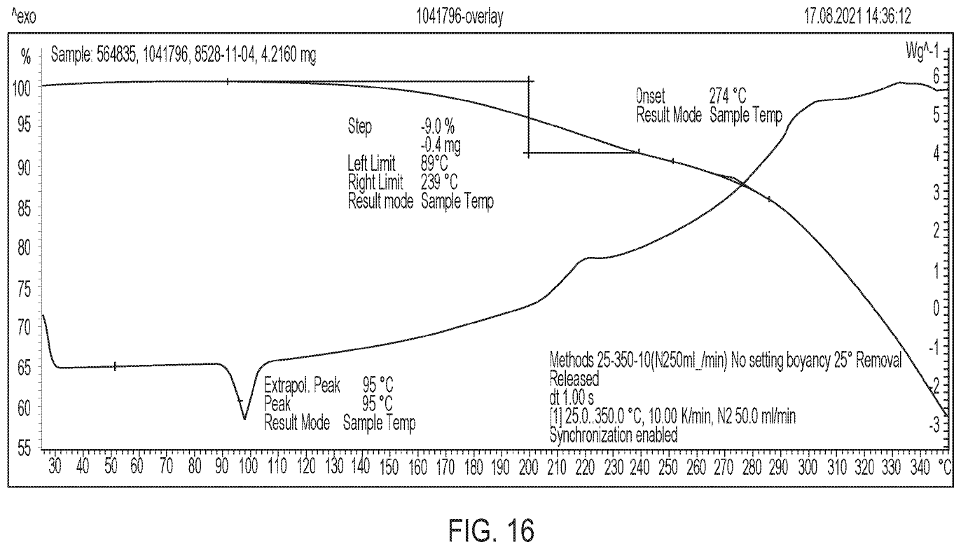

In some embodiments, Compound 1 Form A N-Methylpyrrolidone Solvate is characterized by one or more of the following:
(i) an XRPD pattern substantially similar to that depicted in FIG. 12 (third from top);
(ii) a TGA pattern substantially similar to that depicted in FIG. 16; and
(iii) a DSC pattern substantially similar to that depicted in FIG. 16.

### Compound 1 Form B

In some embodiments, the present disclosure provides Compound 1 as Form B. In some embodiments, Compound 1 Form B is a dimethylsulfoxide (DMSO) solvate.

In some embodiments, Compound 1 Form B is characterized by one or more peaks in its XRPD pattern selected from those at about 8.85, about 11.73., about 14.19, about 17.71, and about 21.61 degrees 2-theta. In some embodiments, Compound 1 Form B is characterized by two or more peaks in its XRPD pattern selected from those at about 8.85, about 11.73, about 14.19, about 17.71, and about 21.61 degrees 2-theta. In some embodiments, Compound 1 Form B is characterized by three or more peaks in its XRPD pattern selected from those at about 8.85, about 11.73, about 14.19, about 17.71, and about 21.61 degrees 2-theta.

In some embodiments, Compound 1 Form B is characterized by peaks in its XRPD pattern at about 8.85, about 11.73, about 14.19, about 17.71, and about 21.61 degrees 2-theta. In some embodiments, Compound 1 Form B is characterized by peaks in its XRPD pattern at substantially all of:

Position ± 0.2 (degrees 2-Theta)

7.09
8.45
8.85
10.28
11.73
12.14
13.07
13.37
13.78
14.19
15.10
15.67
16.07
16.58
16.96
17.71
18.14
18.62
18.85
19.43
19.73
20.16
20.67
21.34
21.61
22.85
23.57
24.11
24.69
24.99
26.30
26.65
28.31

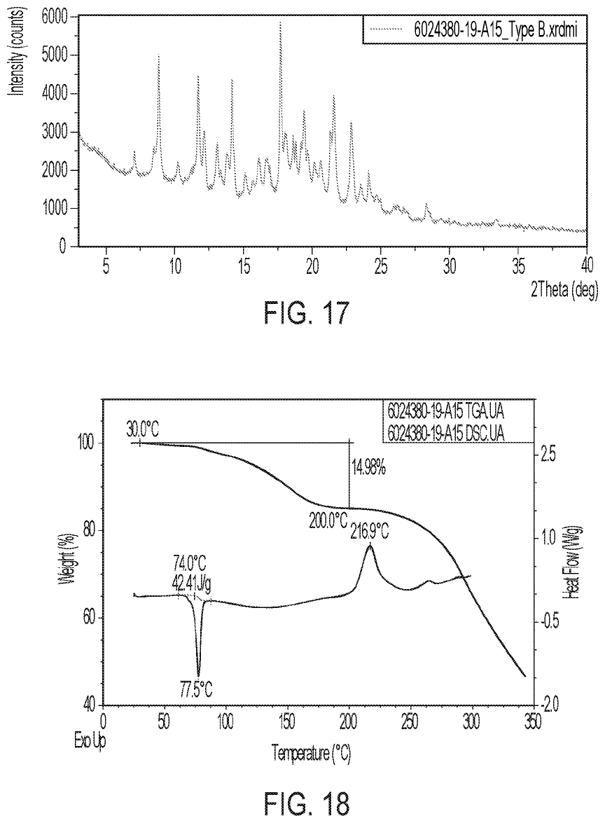

In some embodiments, Compound 1 Form B is characterized by one or more of the following:
(i) an XRPD pattern substantially similar to that depicted in FIG. 17;
(ii) a TGA pattern substantially similar to that depicted in FIG. 18; and
(iii) a DSC pattern substantially similar to that depicted in FIG. 18.

### Salt Forms of Compound 1

In some embodiments, the present disclosure provides complex forms of Compound 1 with a co-former. In some embodiments, the present disclosure provides a complex (otherwise referred to as a “salt” or “salt form”) comprising Compound 1 and a co-former X, designated as Compound 2:

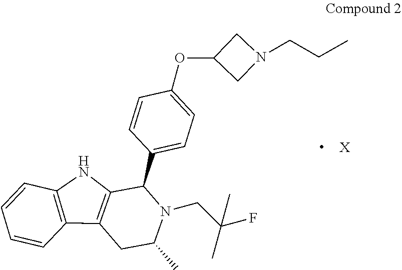

wherein X is a co-former selected from the group consisting of malic acid, fumaric acid, oxalic acid, and phosphoric acid. In some embodiments, X is malic acid. In some embodiments, X is fumaric acid. In some embodiments, X is oxalic acid. In some embodiments, X is phosphoric acid.

It will be appreciated that Compound 2 can exist in a neat or unsolvated form, a hydrated form, a solvated form, and/or a heterosolvated form. In some embodiments, Compound 2 is a neat or unsolvated crystal form and thus does not have any water or solvent incorporated into the crystal structure (and is referred to herein as an “anhydrate” form). In some embodiments, Compound 2 is a hydrated or solvated form. In some embodiments, Compound 2 is a heterosolvated form (e.g., hydrate/solvate form).

In some embodiments, the present disclosure provides Compound 2 as an acetonitrile, ethyl acetate, methyl isobutyl ketone, or tert-butyl acetate solvate.

It will be appreciated that certain solvates and/or hydrates of Compound 2 may be isostructural. For example, as described herein, Compound 2 may exist in a variety of different isostructural solvate forms (e.g., Compound 1 Fumarate Form A Anhydrate, Compound 1 Fumarate Form A Ethyl Acetate Solvate, Compound 1 Fumarate Form D, Compound 1 Fumarate Form F, or Compound 1 Fumarate Form G).

In some embodiments, the term “complex” is used herein to refer to a form comprising Compound 1 non-covalently associated with a co-former (e.g., X). Such non-covalent associations include, by way of example, ionic interactions, dipole-dipole interactions, πt-stacking interactions, hydrogen bond interactions, etc.

It will be appreciated that the term “complex” encompasses salt forms resulting from an ionic interaction between Compound 1 and an acid or base, as well as non-ionic associations between Compound 1 and a neutral species.

In some embodiments, the term “complex” is used herein to refer to a form comprising Compound 1 ionically associated with a co-former (e.g., X). Accordingly, in some such embodiments, the term “complex” is used herein to refer to a salt comprising Compound 1 and an acid or a base.

In some embodiments, a “complex” is an inclusion complex, a salt form, a co-crystal, or a clathrate, or hydrates and/or solvates thereof, etc. In some embodiments, the term “complex” is used to refer to a 1:1 ratio of Compound 1 and co-former (e.g., X). In some embodiments, the term “complex” does not necessarily indicate any particular ratio of Compound 1 to co-former (e.g., X). In some embodiments, a complex is a salt form, or a hydrate or solvate thereof. In some embodiments, a complex is a co-crystal form, or a hydrate or solvate thereof. In some embodiments, a complex is an inclusion complex, or a hydrate or solvate thereof. In some embodiments, a complex is a clathrate, or a hydrate or solvate thereof.

In some embodiments, co-former X and Compound 1 are ionically associated. In some embodiments, Compound 1 is non-covalently associated with co-former X.

A complex form of Compound 1 can exist in a variety of physical forms. For example, a complex form of Compound 1 can be in solution, suspension, or in solid form. In some embodiments, a complex form of Compound 1 is in solution form. In certain embodiments, a complex form of Compound 1 is in solid form. When a complex of Compound 1 is in solid form, said compound may be amorphous, crystalline, or a mixture thereof. In some embodiments, a complex form of Compound 1 is an amorphous solid. In some embodiments, a complex form of Compound 1 is a crystalline solid. Exemplary complex forms of Compound 1 are described in more detail below.

It will be appreciated that Compound 2 (i.e., a complex comprising Compound 1 and a co-former X) can comprise one equivalent of X. Accordingly, in some embodiments, complexes described herein comprise Compound 1 and one equivalent of X. In some embodiments, complexes described herein comprise Compound 1 and two equivalents of X. In some embodiments, complexes described herein comprise Compound 1 and three equivalents of X. In some embodiments, complexes described herein comprise Compound 1 and 0.5-2.5 equivalents of X (e.g., 0.5, 0.9, 1.2, 1.5, etc., equivalents of X).

In some embodiments, the present disclosure provides crystalline solid forms of Compound 2, compositions thereof, and methods of using and preparing crystalline solid forms of Compound 2.

### Compound 1 Malate

In some embodiments, the present disclosure provides a complex form comprising Compound 1 and malic acid (i.e., Compound 2, wherein X is malic acid). In some embodiments, a complex form comprises one equivalent of malic acid. In some embodiments, a complex form comprises two equivalents of malic acid. In some embodiments, the present disclosure provides a crystalline complex form comprising Compound 1 and malic acid. Compound 1 can exist in at least one crystalline malate salt form (“Compound 1 Malate”).

### Compound 1 Malate Form A

In some embodiments, the present disclosure provides Compound 2 as Compound 1 Malate Form A. In some embodiments, Compound 1 Malate Form A comprises a 2:1 ratio of malic acid to Compound 1. In some embodiments, Compound 1 Malate Form A is an anhydrate.

In some embodiments, Compound 1 Malate Form A is characterized by one or more peaks in its XRPD pattern selected from those at about 4.50, about 9.00, about 13.50, about 14.23, and about 18.59 degrees 2-theta. In some embodiments, Compound 1 Malate Form A is characterized by two or more peaks in its XRPD pattern selected from those at about 4.50, about 9.00, about 13.50, about 14.23, and about 18.59 degrees 2-theta. In some embodiments, Compound 1 Malate Form A is characterized by three or more peaks in its XRPD pattern selected from those at about 4.50, about 9.00, about 13.50, about 14.23, and about 18.59 degrees 2-theta.

In some embodiments, Compound 1 Malate Form A is characterized by peaks in its XRPD pattern at about 4.50, about 9.00, about 13.50, about 14.23, and about 18.59 degrees 2-theta. In some embodiments, Compound 1 Malate Form A is characterized by peaks in its XRPD pattern at substantially all of:

Position ± 0.2 (degrees 2-Theta)

4.50
9.00
10.06
12.53
12.73
13.50
14.23
17.91
18.59
19.53
20.18
21.06
22.59
23.06
24.36
27.56

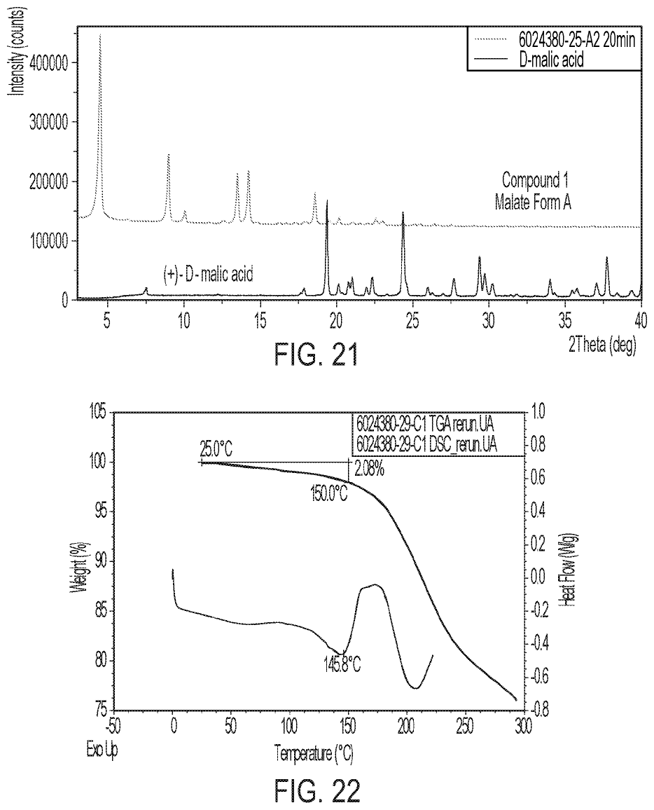

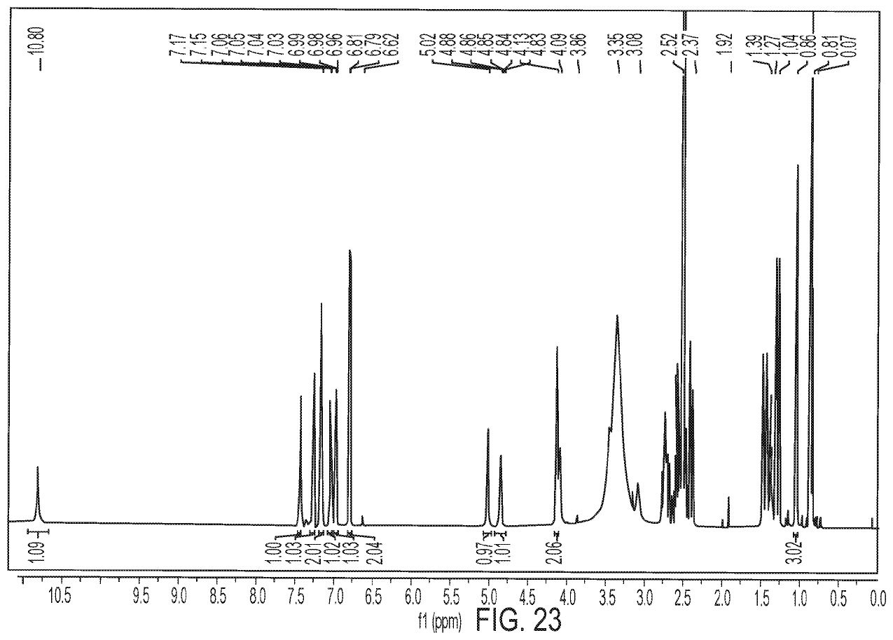

In some embodiments, Compound 1 Malate Form A is characterized by one or more of the following:
(i) an XRPD pattern substantially similar to that depicted in FIG. 21;
(ii) a TGA pattern substantially similar to that depicted in FIG. 22; and
(iii) a DSC pattern substantially similar to that depicted in FIG. 22.

### Compound 1 Fumarate

In some embodiments, the present disclosure provides a complex form comprising Compound 1 and fumaric acid (i.e., Compound 2, wherein X is fumaric acid). In some embodiments, a complex form comprises one equivalent of fumaric acid. In some embodiments, the present disclosure provides a crystalline complex form comprising Compound 1 and fumaric acid. Compound 1 can exist in several crystalline fumarate salt forms (“Compound 1 Fumarate”).

### Compound 1 Fumarate Form A Anhydrate

In some embodiments, the present disclosure provides Compound 2 as Compound 1 Fumarate Form A Anhydrate. In some embodiments, Compound 1 Fumarate Form A Anhydrate comprises a 1:1 ratio of fumaric acid to Compound 1. In some embodiments, Compound 1 Fumarate Form A is an anhydrate. In some embodiments, Compound 1 Fumarate Form A Anhydrate is an unsolvated anhydrate.

In some embodiments, Compound 1 Fumarate Form A Anhydrate is characterized by one or more peaks in its XRPD pattern selected from those at about 5.77, about 8.23, about 9.25, about 11.47, about 12.50, about 15.28, and about 17.23 degrees 2-theta. In some embodiments, Compound 1 Fumarate Form A Anhydrate is characterized by two or more peaks in its XRPD pattern selected from those at about 5.77, about 8.23, about 9.25, about 11.47, about 12.50, about 15.28, and about 17.23 degrees 2-theta. In some embodiments, Compound 1 Fumarate Form A Anhydrate is characterized by three or more peaks in its XRPD pattern selected from those at about 5.77, about 8.23, about 9.25, about 11.47, about 12.50, about 15.28, and about 17.23 degrees 2-theta.

In some embodiments, Compound 1 Fumarate Form A Anhydrate is characterized by peaks in its XRPD pattern at about 5.77, about 8.23, about 9.25, about 11.47, about 12.50, about 15.28, and about 17.23 degrees 2-theta. In some embodiments, Compound 1 Fumarate Form A Anhydrate is characterized by peaks in its XRPD pattern at substantially all of:

Position ± 0.2 (degrees 2-Theta)

5.77
8.23
9.25
11.47
12.50
13.20
13.54
13.95
14.16
15.28
16.39
17.23
17.95
18.40
19.24
19.53
19.98
20.39
21.04
22.58
23.18
24.19
24.76
25.15
25.77
26.47
27.91
28.90
29.51
34.92
38.93

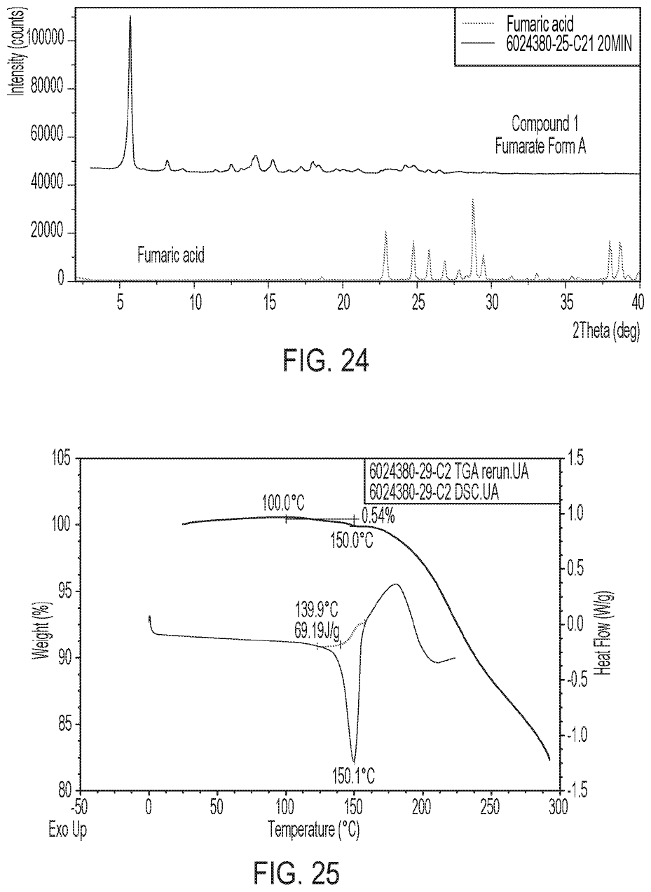

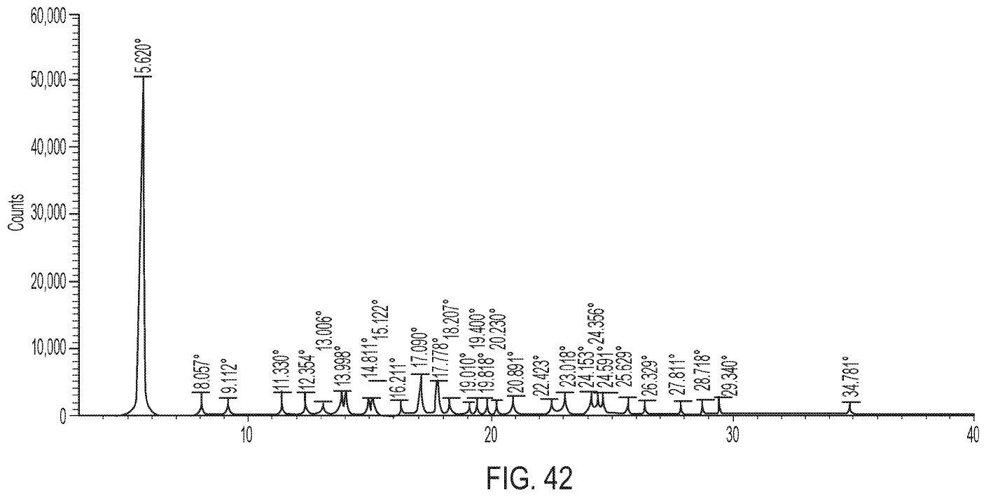

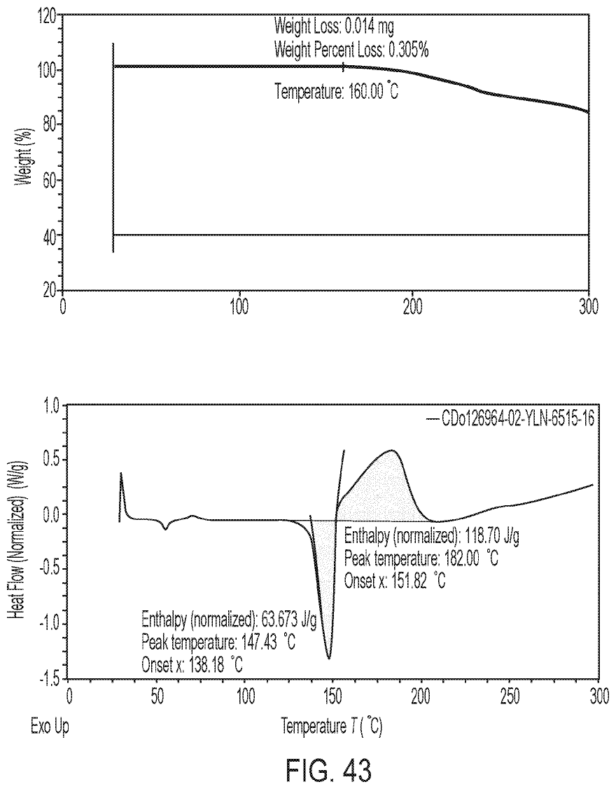

In some embodiments, Compound 1 Fumarate Form A Anhydrate is characterized by one or more of the following:
(i) an XRPD pattern substantially similar to that depicted in FIG. 24 and/or FIG. 42;
(ii) a TGA pattern substantially similar to that depicted in FIG. 25 and/or FIG. 43; and
(iii) a DSC pattern substantially similar to that depicted in FIG. 25 and/or FIG. 43.

### Compound 1 Fumarate Form A Ethyl Acetate Solvate

In some embodiments, the present disclosure provides Compound 2 as Compound 1 Fumarate Form A Ethyl Acetate Solvate. In some embodiments, Compound 1 Fumarate Form A Ethyl Acetate Solvate comprises a 1:1 ratio of fumaric acid to Compound 1. In some embodiments, Compound 1 Fumarate Form A is an ethyl acetate solvate.

In some embodiments, Compound 1 Fumarate Form A Ethyl Acetate Solvate is characterized by one or more peaks in its XRPD pattern selected from those at about 4.94, about 8.18, about 14.14, about 15.95, about 16.38, and about 18.54 degrees 2-theta. In some embodiments, Compound 1 Fumarate Form A Ethyl Acetate Solvate is characterized by two or more peaks in its XRPD pattern selected from those at about 4.94, about 8.18, about 14.14, about 15.95, about 16.38, and about 18.54 degrees 2-theta. In some embodiments, Compound 1 Fumarate Form A Ethyl Acetate Solvate is characterized by three or more peaks in its XRPD pattern selected from those at about 4.94, about 8.18, about 14.14, about 15.95, about 16.38, and about 18.54 degrees 2-theta.

In some embodiments, Compound 1 Fumarate Form A Ethyl Acetate Solvate is characterized by peaks in its XRPD pattern selected from those at about 4.94, about 8.18, about 14.14, about 15.95. about 16.38, and about 18.54 degrees 2-theta. In some embodiments, Compound 1 Fumarate Form A Ethyl Acetate Solvate is characterized by peaks in its XRPD pattern at substantially all of:

Position ± 0.2 (degrees 2-Theta)

4.94
8.18
11.85
12.54
14.14
14.68
14.96
15.95
16.38
17.25
18.12
18.54
19.86
20.71

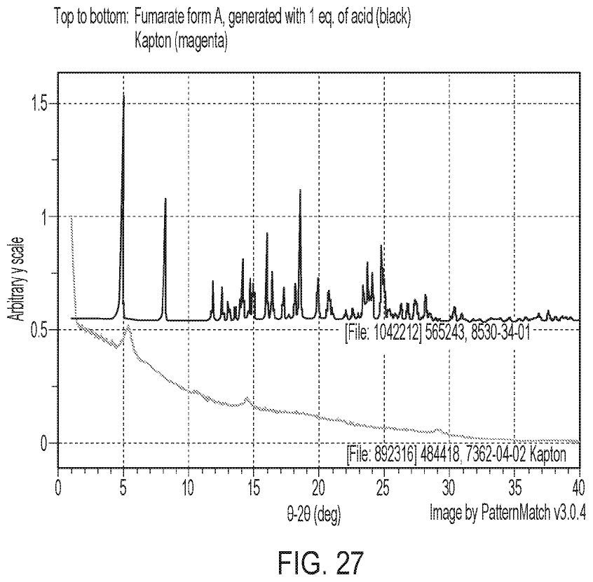

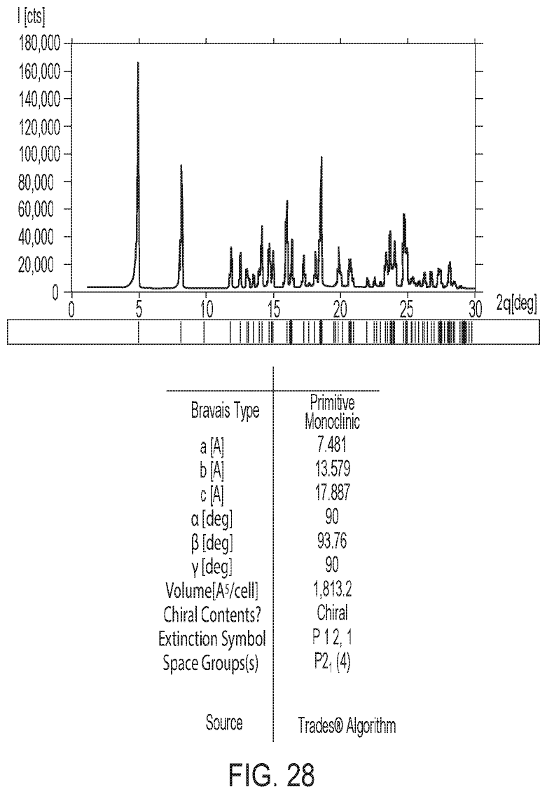

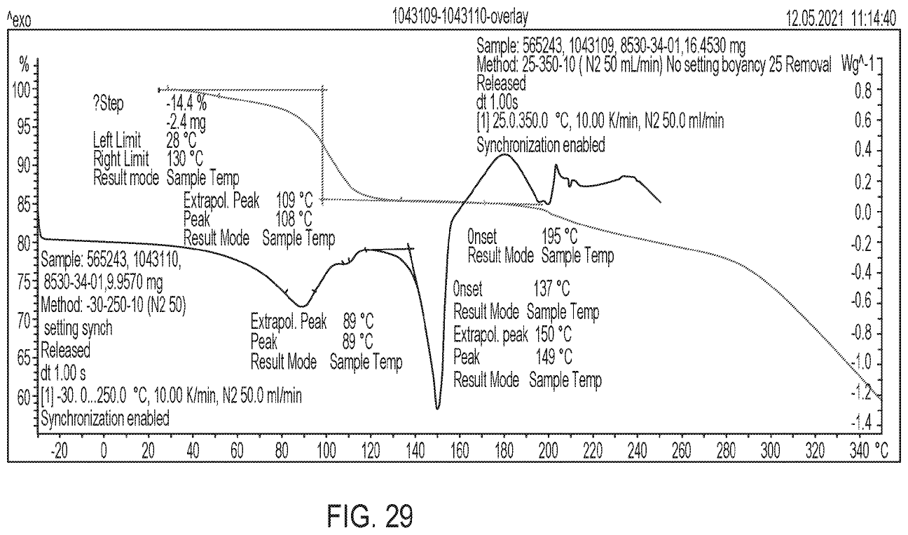

In some embodiments, Compound 1 Fumarate Form A Ethyl Acetate Solvate is characterized by one or more of the following:
(i) an XRPD pattern substantially similar to that depicted in FIG. 27 and/or FIG. 28;
(ii) a TGA pattern substantially similar to that depicted in FIG. 29; and
(iii) a DSC pattern substantially similar to that depicted in FIG. 29.

### Compound 1 Fumarate Form C

In some embodiments, the present disclosure provides Compound 2 as Compound 1 Fumarate Form C. In some embodiments, Compound 1 Fumarate Form C is hydrate.

In some embodiments, Compound 1 Fumarate Form C is characterized by one or more peaks in its XRPD pattern selected from those at about 5.67, about 8.14, about 12.40, about 13.84, about 14.04, and about 15.16 degrees 2-theta. In some embodiments, Compound 1 Fumarate Form C is characterized by two or more peaks in its XRPD pattern selected from those at about 5.67, about 8.14, about 12.40, about 13.84, about 14.04, and about 15.16 degrees 2-theta. In some embodiments, Compound 1 Fumarate Form C is characterized by three or more peaks in its XRPD pattern selected from those at about 5.67, about 8.14, about 12.40, about 13.84, about 14.04, and about 15.16 degrees 2-theta.

In some embodiments, Compound 1 Fumarate Form C is characterized by peaks in its XRPD pattern at about 5.67, about 8.14, about 12.40, about 13.84, about 14.04, and about 15.16, degrees 2-theta. In some embodiments, Compound 1 Fumarate Form C is characterized by peaks in its XRPD pattern at substantially all of:

Position ± 0.2 (degrees 2-Theta)

5.67
6.52
8.14
9.14
11.38
11.95
12.40
13.07
13.44
13.84
14.04
15.16
17.82
19.45

In some embodiments, Compound 1 Fumarate Form C is characterized by an XRPD pattern substantially similar to that depicted in FIG. 31.

### Compound 1 Fumarate Form D

In some embodiments, the present disclosure provides Compound 2 as Compound 1 Fumarate Form D. In some embodiments, Compound 1 Fumarate Form D comprises a 1:1 ratio of fumaric acid to Compound 1. In some embodiments, Compound 1 Fumarate Form D is a methyl isobutyl ketone (MIBK) solvate.

In some embodiments, Compound 1 Fumarate Form D is characterized by one or more peaks in its XRPD pattern selected from those at about 4.84, about 8.08, about 14.08, about 15.73, and about 18.40, degrees 2-theta. In some embodiments, Compound 1 Fumarate Form D is characterized by two or more peaks in its XRPD pattern selected from those at about 4.84, about 8.08, about 14.08, about 15.73, and about 18.40, degrees 2-theta. In some embodiments, Compound 1 Fumarate Form D is characterized by three or more peaks in its XRPD pattern selected from those at about 4.84, about 8.08, about 14.08, about 15.73, and about 18.40, degrees 2-theta.

In some embodiments, Compound 1 Fumarate Form D is characterized by peaks in its XRPD pattern at about 4.84, about 8.08, about 14.08, about 15.73, and about 18.40, degrees 2-theta. In some embodiments, Compound 1 Fumarate Form D is characterized by peaks in its XRPD pattern at substantially all of:

Position ± 0.2 (degrees 2-Theta)

4.84
8.08
12.49
13.46
14.08
14.09
14.11
14.54
14.83
15.73
16.18
17.02
18.40
20.41

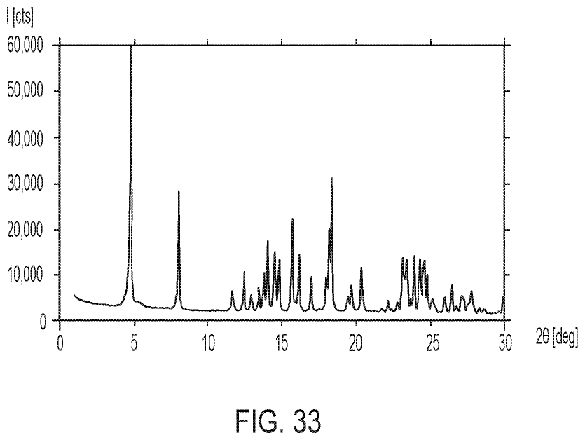

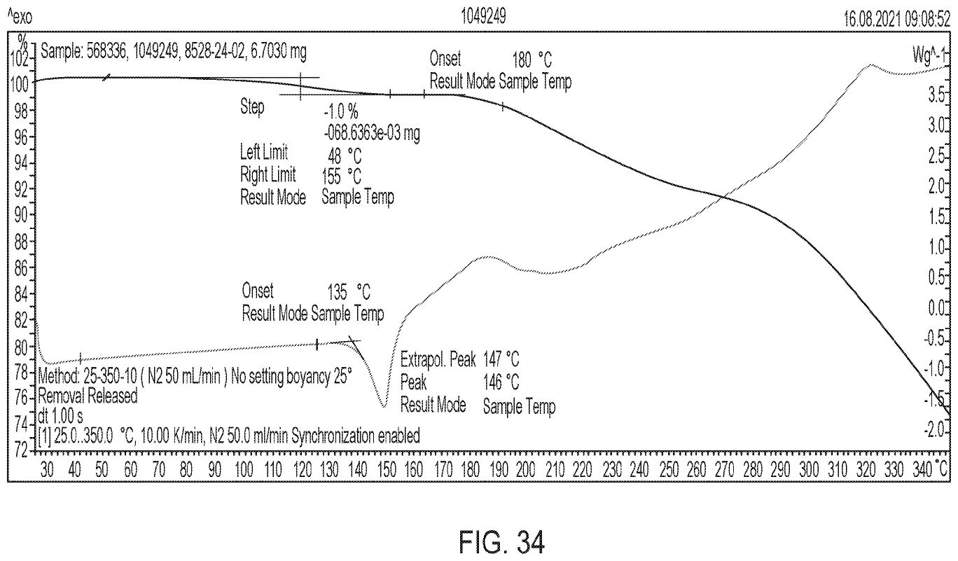

In some embodiments, Compound 1 Fumarate Form D is characterized by one or more of the following:
(i) an XRPD pattern substantially similar to that depicted in FIG. 33;
(ii) a TGA pattern substantially similar to that depicted in FIG. 34; and
(iii) a DSC pattern substantially similar to that depicted in FIG. 34.

### Compound 1 Fumarate Form E

In some embodiments, the present disclosure provides Compound 2 as Compound 1 Fumarate Form E. In some embodiments, Compound 1 Fumarate Form E comprises a 1:1 ratio of fumaric acid to Compound 1. In some embodiments, Compound 1 Fumarate Form E is an anhydrate.

In some embodiments, Compound 1 Fumarate Form E is characterized by one or more peaks in its XRPD pattern selected from those at about 5.83, about 7.03, about 8.69, about 12.88, about 13.43, about 14.68, about 15.65, about 16.65, and about 18.46 degrees 2-theta. In some embodiments, Compound 1 Fumarate Form E is characterized by two or more peaks in its XRPD pattern selected from those at about 5.83, about 7.03, about 8.69, about 12.88, about 13.43, about 14.68, about 15.65, about 16.65, and about 18.46 degrees 2-theta. In some embodiments, Compound 1 Fumarate Form E is characterized by three or more peaks in its XRPD pattern selected from those at about 5.83, about 7.03, about 8.69, about 12.88, about 13.43, about 14.68, about 15.65, about 16.65, and about 18.46 degrees 2-theta.

In some embodiments, Compound 1 Fumarate Form E is characterized by peaks in its XRPD pattern at about 5.83, about 7.03, about 8.69, about 12.88, about 13.43, about 14.68, about 15.65, about 16.65, and about 18.46 degrees 2-theta. In some embodiments, Compound 1 Fumarate Form E is characterized by peaks in its XRPD pattern at substantially all of:

Position ± 0.2 (degrees 2-Theta)

5.83
7.03
8.69
10.91
12.88
13.43
14.68
15.65
16.08
16.65
17.72
18.46
18.88
19.63
19.99
21.73
22.00
22.31
23.83
24.63
25.02
26.48
27.37
28.60
29.32
29.81
30.05
35.69
39.70
40.32
40.97

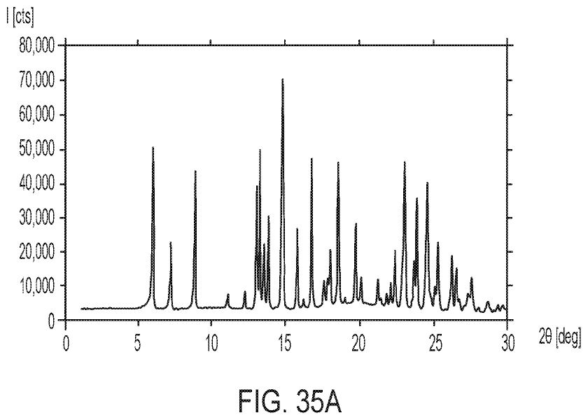

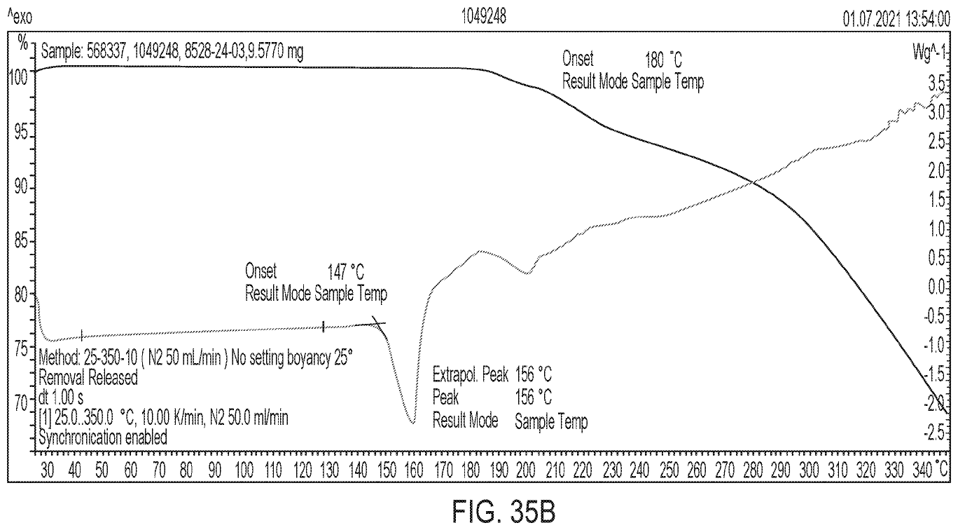

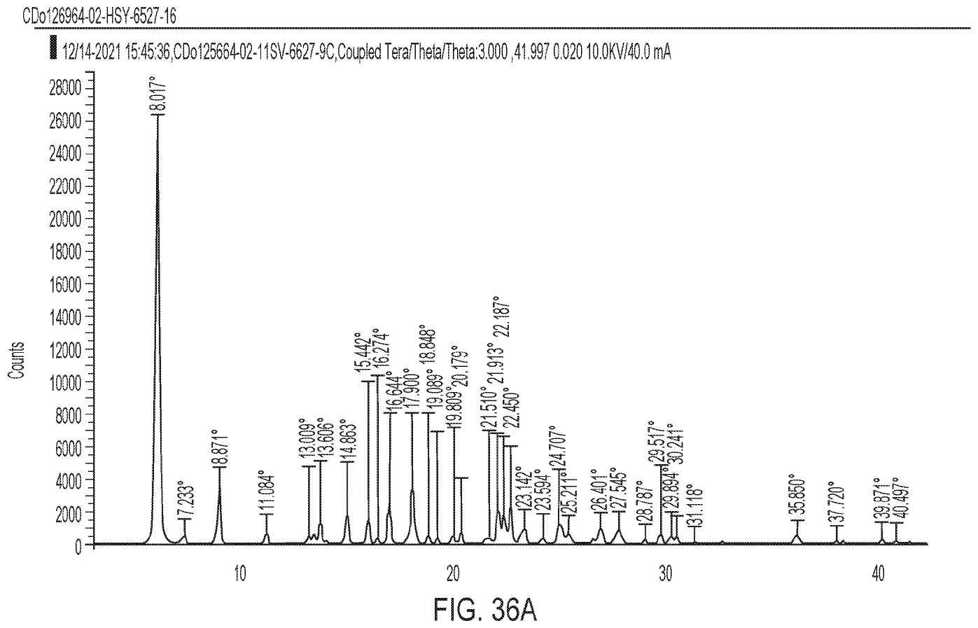

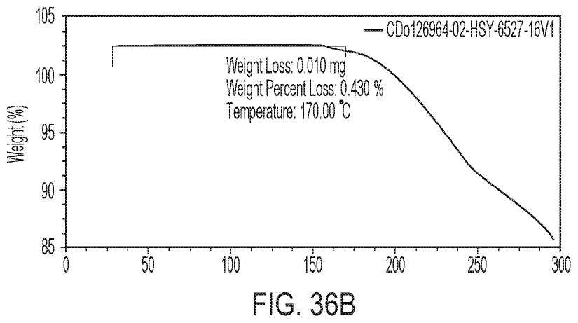

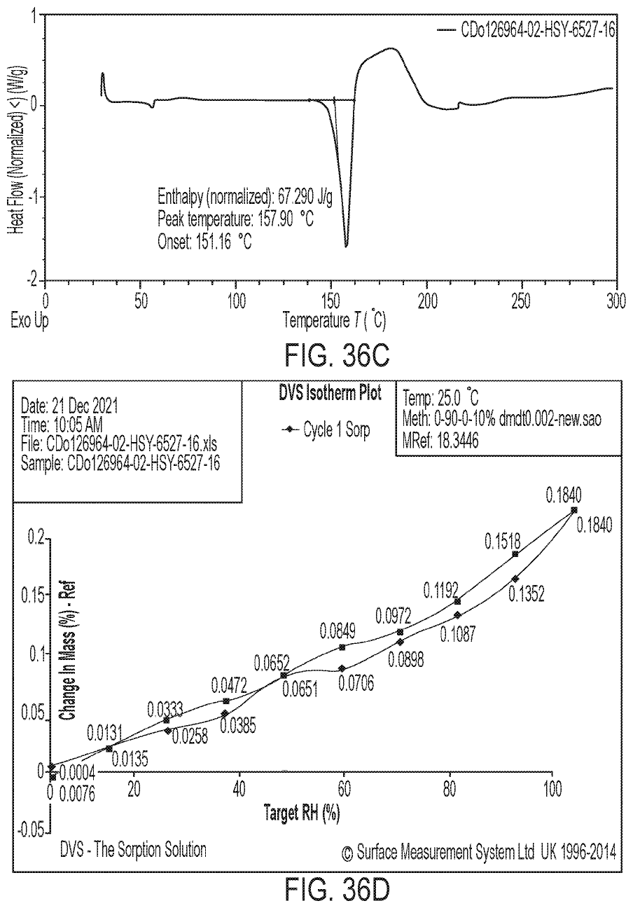

In some embodiments, Compound 1 Fumarate Form E is characterized by one or more of the following:
(i) an XRPD pattern substantially similar to that depicted in FIG. 35A and/or FIG. 36A;
(ii) a TGA pattern substantially similar to that depicted in FIG. 35B and/or FIG. 36B; and
(iii) a DSC pattern substantially similar to that depicted in FIG. 35B and/or FIG. 36C.

### Compound 1 Fumarate Form F

In some embodiments, the present disclosure provides Compound 2 as Compound 1 Fumarate Form F. In some embodiments, Compound 1 Fumarate Form F comprises a 1:1 ratio of fumaric acid to Compound 1. In some embodiments, Compound 1 Fumarate Form F is a tert-butyl acetate (tBuOAc) solvate.

In some embodiments, Compound 1 Fumarate Form F is characterized by one or more peaks in its XRPD pattern selected from those at about 4.77, about 8.03, about 14.56, about 15.90, about 17.82, and about 24.77 degrees 2-theta. In some embodiments, Compound 1 Fumarate Form F is characterized by two or more peaks in its XRPD pattern selected from those at about 4.77, about 8.03, about 14.56, about 15.90, about 17.82, and about 24.77 degrees 2-theta. In some embodiments, Compound 1 Fumarate Form F is characterized by three or more peaks in its XRPD pattern selected from those at about 4.77, about 8.03, about 14.56, about 15.90, about 17.82, and about 24.77 degrees 2-theta.

In some embodiments, Compound 1 Fumarate Form F is characterized by peaks in its XRPD pattern at about 4.77, about 8.03, about 14.56, about 15.90, about 17.82, and about 24.77 degrees 2-theta. In some embodiments, Compound 1 Fumarate Form F is characterized by peaks in its XRPD pattern at substantially all of:

Position ± 0.2 (degrees 2-Theta)

4.77
8.03
11.54
12.91
13.49
13.98
14.56
15.90
17.17
17.82
20.53
23.10
23.52
24.77

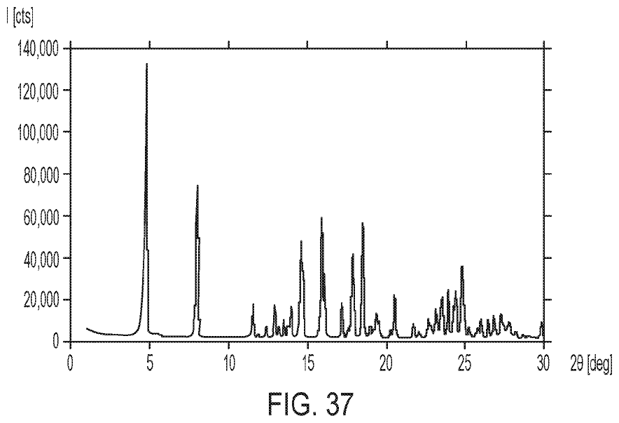

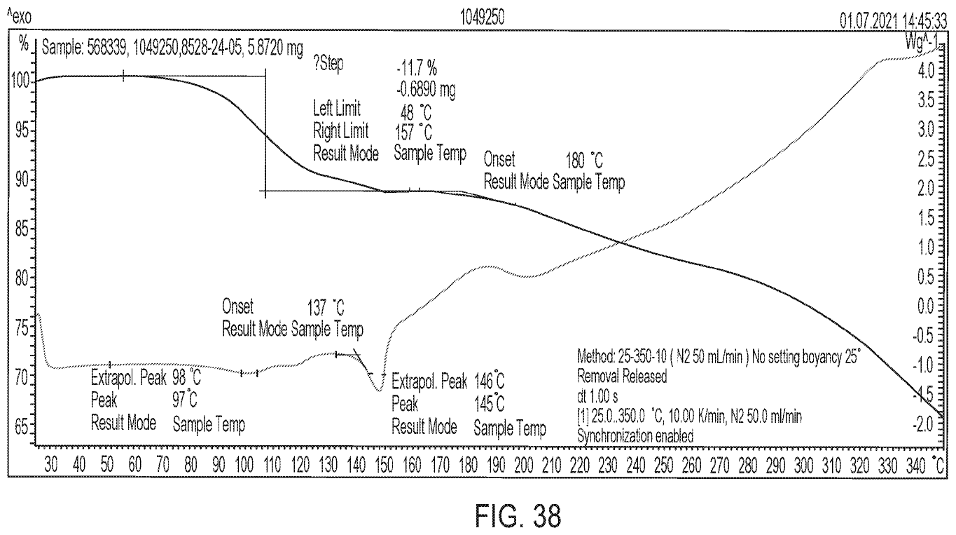

In some embodiments, Compound 1 Fumarate Form F is characterized by one or more of the following:
(i) an XRPD pattern substantially similar to that depicted in FIG. 37;
(ii) a TGA pattern substantially similar to that depicted in FIG. 38; and
(iii) a DSC pattern substantially similar to that depicted in FIG. 38.

### Compound 1 Fumarate Form G

In some embodiments, the present disclosure provides Compound 2 as Compound 1 Fumarate Form G. In some embodiments, Compound 1 Fumarate Form G comprises a 1:1 ratio of fumaric acid to Compound 1. In some embodiments, Compound 1 Fumarate Form G is an acetonitrile solvate.

In some embodiments, Compound 1 Fumarate Form G is characterized by one or more peaks in its XRPD pattern selected from those at about 5.49, about 8.43, about 12.74, about 13.69, and about 19.56 degrees 2-theta. In some embodiments, Compound 1 Fumarate Form G is characterized by two or more peaks in its XRPD pattern selected from those at about 5.49, about 8.43, about 12.74, about 13.69, and about 19.56 degrees 2-theta. In some embodiments, Compound 1 Fumarate Form G is characterized by three or more peaks in its XRPD pattern selected from those at about 5.49, about 8.43, about 12.74, about 13.69, and about 19.56 degrees 2-theta.

In some embodiments, Compound 1 Fumarate Form G is characterized by peaks in its XRPD pattern at about 5.49, about 8.43, about 12.74, about 13.69, and about 19.56 degrees 2-theta. In some embodiments, Compound 1 Fumarate Form G is characterized by peaks in its XRPD pattern at substantially all of:

Position ± 0.2 (degrees 2-Theta)

5.49
8.43
12.10
12.74
13.69
14.23
14.71
17.67
19.56
20.98

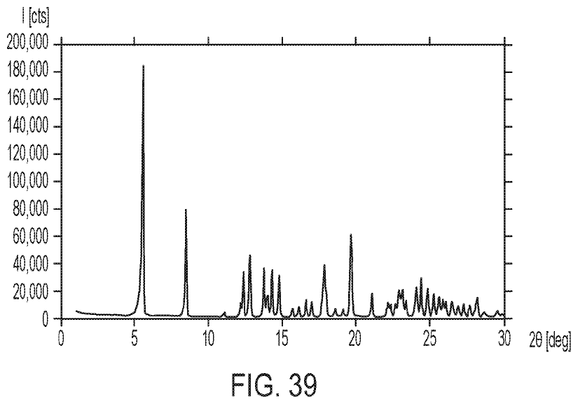

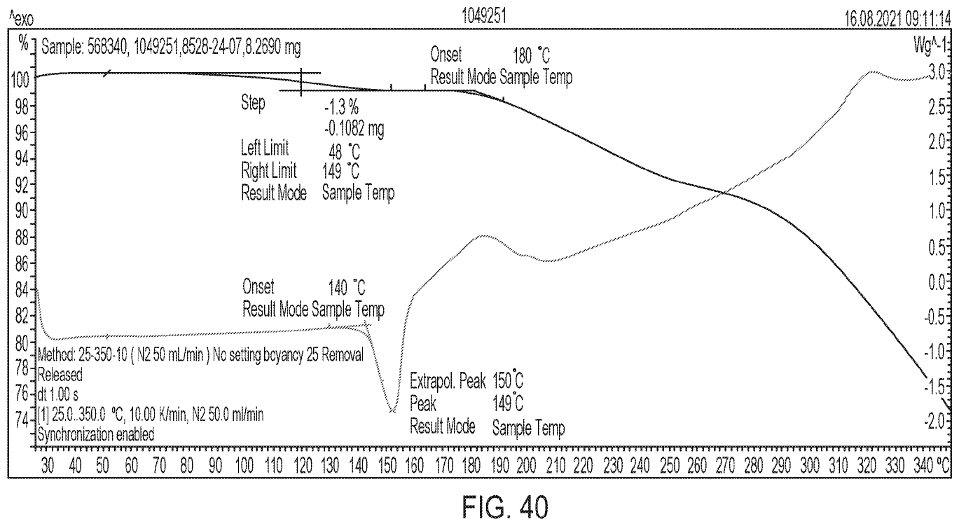

In some embodiments, Compound 1 Fumarate Form G is characterized by one or more of the following:
(i) an XRPD pattern substantially similar to that depicted in FIG. 39;
(ii) a TGA pattern substantially similar to that depicted in FIG. 40; and
(iii) a DSC pattern substantially similar to that depicted in FIG. 40.

### Compound 1 Fumarate Form J

In some embodiments, the present disclosure provides Compound 2 as Compound 1 Fumarate Form J.

In some embodiments, Compound 1 Fumarate Form J is characterized by one or more peaks in its XRPD pattern selected from those at about 8.28, about 9.18, about 9.53, about 9.77, about 18.66, and about 19.19 degrees 2-theta. In some embodiments, Compound 1 Fumarate Form J is characterized by two or more peaks in its XRPD pattern selected from those at about 8.28, about 9.18, about 9.53, about 9.77, about 18.66, and about 19.19 degrees 2-theta. In some embodiments, Compound 1 Fumarate Form J is characterized by three or more peaks in its XRPD pattern selected from those at about 8.28, about 9.18, about 9.53, about 9.77, about 18.66, and about 19.19 degrees 2-theta.

In some embodiments, Compound 1 Fumarate Form J is characterized by peaks in its XRPD pattern at about 8.28, about 9.18, about 9.53, about 9.77, about 18.66, and about 19.19 degrees 2-theta. In some embodiments, Compound 1 Fumarate Form J is characterized by peaks in its XRPD pattern at substantially all of:

Position ± 0.2 (degrees 2-Theta)

4.37
5.34
8.28
9.18
9.53
9.77
11.79
13.81
17.03
18.66
19.19

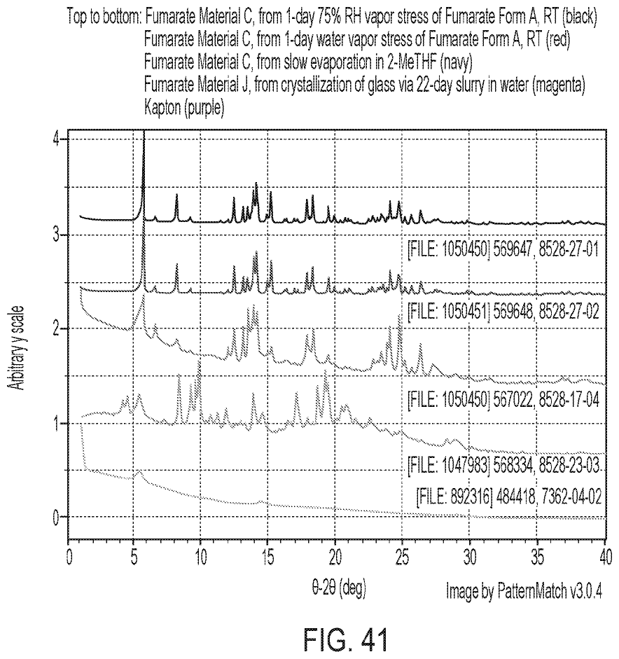

In some embodiments, Compound 1 Fumarate Form J is characterized by an XRPD pattern substantially similar to that depicted in FIG. 41.

### Compound 1 Oxalate

In some embodiments, the present disclosure provides a complex form comprising Compound 1 and oxalic acid (i.e., Compound 2, wherein X is oxalic acid). In some embodiments, the present disclosure provides a crystalline complex form comprising Compound 1 and oxalic acid. Compound 1 can exist at least one crystalline oxalate salt form (“Compound 1 Oxalate”).

### Compound 1 Oxalate Form A

In some embodiments, the present disclosure provides Compound 2 as Compound 1 Oxalate Form A. In some embodiments, Compound 1 Oxalate Form A is an anhydrate.

In some embodiments, Compound 1 Oxalate Form A is characterized by one or more peaks in its XRPD pattern selected from those at about 4.68, about 8.95, about 9.37, about 12.76, about 14.07, about 18.78 degrees 2-theta. In some embodiments, Compound 1 Oxalate Form A is characterized by two or more peaks in its XRPD pattern selected from those at about 4.68, about 8.95, about 9.37, about 12.76, about 14.07, about 18.78 degrees 2-theta. In some embodiments, Compound 1 Oxalate Form A is characterized by three or more peaks in its XRPD pattern selected from those at about 4.68, about 8.95, about 9.37, about 12.76, about 14.07, about 18.78 degrees 2-theta.

In some embodiments, Compound 1 Oxalate Form A is characterized by peaks in its XRPD pattern at about 4.68, about 8.95, about 9.37, about 12.76, about 14.07, about 18.78 degrees 2-theta. In some embodiments, Compound 1 Oxalate Form A is characterized by peaks in its XRPD pattern at substantially all of:

Position ± 0.2 (degrees 2-Theta)

4.68
8.95
9.37
9.83
11.14
11.58
12.76
14.07
14.47
16.54
18.78
19.67
20.92
21.45
22.90
23.55
28.32

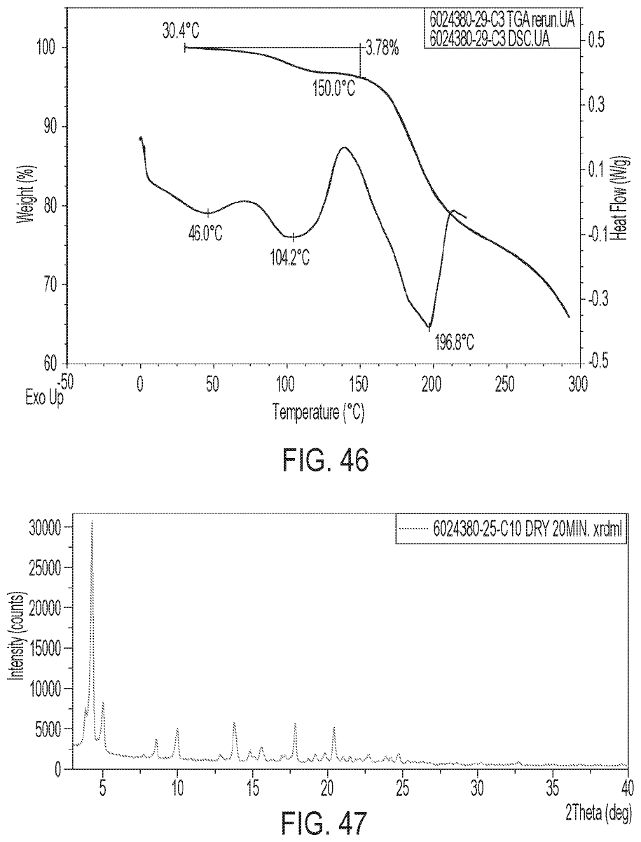

In some embodiments, Compound 1 Oxalate Form A is characterized by one or more of the following:
(i) an XRPD pattern substantially similar to that depicted in FIG. 45;
(ii) a TGA pattern substantially similar to that depicted in FIG. 46; and
(iii) a DSC pattern substantially similar to that depicted in FIG. 46.

### Compound 1 Phosphate

In some embodiments, the present disclosure provides a complex form comprising Compound 1 and phosphoric acid (i.e., Compound 2, wherein X is phosphoric acid). In some embodiments, the present disclosure provides a crystalline complex form comprising Compound 1 and phosphoric acid. Compound 1 can exist at least two crystalline phosphate salt forms (“Compound 1 Phosphate”).

### Compound 1 Phosphate Form A

In some embodiments, the present disclosure provides Compound 2 as Compound 1 Phosphate Form A. In some embodiments, Compound 1 Phosphate Form A is a hydrate.

In some embodiments, Compound 1 Phosphate Form A is characterized by one or more peaks in its XRPD pattern selected from those at about 3.86, about 4.30, about 5.05, about 13.76, about 17.85, and about 20.42 degrees 2-theta. In some embodiments, Compound 1 Phosphate Form A is characterized by two or more peaks in its XRPD pattern selected from those at about 3.86, about 4.30, about 5.05, about 13.76, about 17.85, and about 20.42 degrees 2-theta. In some embodiments, Compound 1 Phosphate Form A is characterized by three or more peaks in its XRPD pattern selected from those at about 3.86, about 4.30, about 5.05, about 13.76, about 17.85, and about 20.42 degrees 2-theta.

In some embodiments, Compound 1 Phosphate Form A is characterized by peaks in its XRPD pattern at about 3.86, about 4.30, about 5.05, about 13.76, about 17.85, and about 20.42 degrees 2-theta. In some embodiments, Compound 1 Phosphate Form A is characterized by peaks in its XRPD pattern at substantially all of:

Position ± 0.2 (degrees 2-Theta)

3.86
4.30
5.05
7.72
8.57
10.01
12.84
13.76
14.82
15.57
16.93
17.20
17.85
18.71
19.17
19.82
20.42
21.04
21.49
21.85
22.17
22.76
23.89
24.24
24.77
25.34
25.81
26.35
30.26
32.75

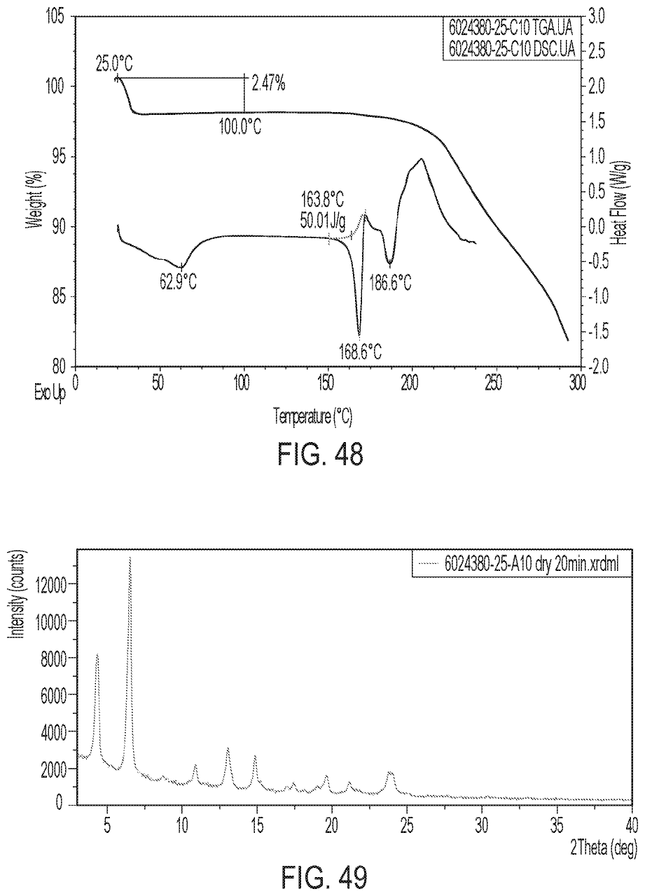

In some embodiments, Compound 1 Phosphate Form A is characterized by one or more of the following:
(i) an XRPD pattern substantially similar to that depicted in FIG. 47;
(ii) a TGA pattern substantially similar to that depicted in FIG. 48; and
(iii) a DSC pattern substantially similar to that depicted in FIG. 48.

### Compound 1 Phosphate Form B

In some embodiments, the present disclosure provides Compound 2 as Compound 1 Phosphate Form B.

In some embodiments, Compound 1 Phosphate Form B is characterized by one or more peaks in its XRPD pattern selected from those at about 4.38, about 6.54, about 13.07, about 14.89, and about 23.76 degrees 2-theta. In some embodiments, Compound 1 Phosphate Form B is characterized by two or more peaks in its XRPD pattern selected from those at about 4.38, about 6.54, about 13.07, about 14.89, and about 23.76 degrees 2-theta. In some embodiments, Compound 1 Phosphate Form B is characterized by three or more peaks in its XRPD pattern selected from those at about 4.38, about 6.54, about 13.07, about 14.89, and about 23.76 degrees 2-theta.

In some embodiments, Compound 1 Phosphate Form B is characterized by peaks in its XRPD pattern at about 4.38, about 6.54, about 13.07, about 14.89, and about 23.76 degrees 2-theta. In some embodiments, Compound 1 Phosphate Form B is characterized by peaks in its XRPD pattern at substantially all of:

Position ± 0.2 (degrees 2-Theta)

3.29
4.38
6.54
7.72
8.70
9.04
10.93
13.07
14.89
16.94
17.47
19.70
21.18
23.76
24.10
25.08

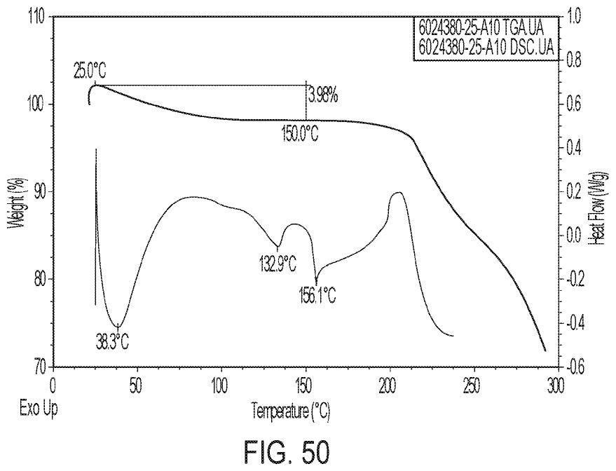

In some embodiments, Compound 1 Phosphate Form B is characterized by one or more of the following:
(i) an XRPD pattern substantially similar to that depicted in FIG. 49;
(ii) a TGA pattern substantially similar to that depicted in FIG. 50; and
(iii) a DSC pattern substantially similar to that depicted in FIG. 50.

### Preparing Provided Forms

In some embodiments, the present disclosure provides methods of preparing Compound 2 (i.e., salt forms of Compound 1), as well as solid forms of Compound 1 and Compound 2. The present disclosure also provides methods of preparing compositions comprising mixtures of Compound 1 and/or Compound 2 in one or more solid forms and/or an amorphous form.

### Compound 1

In some embodiments, solid forms of Compound 1 can be prepared by dissolving Compound 1 (e.g., amorphous Compound 1, crystalline Compound 1, or a mixture thereof) in a suitable solvent and then causing Compound 1 to return to the solid phase. In some embodiments, solid forms of Compound 1 are prepared by combining Compound 1 (e.g., amorphous Compound 1, crystalline Compound 1, or a mixture thereof) in a suitable solvent under suitable conditions and isolating a solid form of Compound 1.

In some embodiments, a suitable solvent is selected from acetone, acetonitrile, dimethylsulfoxide, dioxane, ethyl acetate, N-methylpyrrolidone, tetrahydrofuran, and water, or any combination thereof.

In some embodiments, a method of preparing a solid form of Compound 1 comprises a step of heating a mixture comprising Compound 1 to a suitable temperature (e.g., from about 30° C. to about 60° C.). In some embodiments, a method of preparing a solid form of Compound 1 comprises a step of stirring a mixture comprising Compound 1 at ambient temperature. In some embodiments, a method of preparing a solid form of Compound 1 comprises a step of cooling a mixture comprising Compound 1 to a suitable temperature (e.g., from about −20° C. to about 0° C.).

In some embodiments, a solid form of Compound 1 precipitates from a mixture (e.g., a solution, suspension, or slurry). In some embodiments, Compound 1 crystallizes from a solution. In some embodiments, Compound 1 crystallizes from a solution following seeding of the solution (e.g., adding crystals of Compound 1 to the solution). In some embodiments, Compound 1 precipitates or crystallizes from a mixture after cooling, addition of an anti-solvent, and/or removal of all or part of a solvent through methods such as evaporation, distillation, filtration, reverse osmosis, absorption, or reaction.

In some embodiments, a method of preparing a solid form of Compound 1 comprises a step of isolating the solid form of Compound 1. It will he appreciated that a solid form of Compound 1 may be isolated by any suitable means. In some embodiments, a solid form of Compound 1 is separated from a supernatant by filtration. In some embodiments, a solid form of Compound 1 is separate from a supernatant by decanting.

In some embodiments, an isolated solid form of Compound 1 is dried (e.g., in air or under reduced pressure, optionally at elevated temperature).

In some embodiments, a solid form of Compound 1 is prepared by converting one solid form of Compound 1 into another solid form of Compound 1.

### Compound 2

In some embodiments, Compound 2 (e.g., amorphous Compound 2, crystalline Compound 2, or a mixture thereof) is prepared by contacting Compound 1 (e.g., amorphous Compound 1, crystalline Compound 1, or a mixture thereof) with a suitable acid, such as malic acid, fumaric acid, oxalic acid, or phosphoric acid. In some embodiments, the present disclosure provides a method of preparing Compound 2 comprising steps of providing Compound 1; and combining Compound 1 with a suitable acid, optionally in a suitable solvent, to provide Compound 2. In some embodiments, about 1.0, about 1.1, about 1.2, or about 2.0 equivalents of suitable acid are added.

In some embodiments, a solid form of Compound 2 is prepared by dissolving Compound 2 (e.g., amorphous Compound 2, crystalline Compound 2, or a mixture thereof) in a suitable solvent and then causing Compound 2 to return to the solid phase. In some embodiments, a solid form of Compound 2 is prepared by combining Compound 2 (e.g., amorphous Compound 2, crystalline Compound 2, or a mixture thereof) in a suitable solvent under suitable conditions and isolating the solid form of Compound 2.

In some embodiments, a suitable solvent is selected from acetone, acetonitrile, 2-butanol, dichloroethane, dioxane, ethanol, ethyl acetate, heptane, isopropanol, 2-methyltetrahydrofuran, methyl ethyl ketone, methyl isobutyl ketone, methyl tert-butyl ether, N-methylpyrrolidone, tert-butyl acetate, toluene, and water, or any combination thereof.

In some embodiments, a method of preparing Compound 2 (e.g., a solid form of Compound 2) comprises a step of heating a mixture comprising Compound 2 to a suitable temperature (e.g., from about 30° C. to about 60° C.). In some embodiments, a method of preparing Compound 2 (e.g., a solid form of Compound 2) comprises a step of stirring a mixture comprising Compound 2 at ambient temperature. In some embodiments, a method of preparing Compound 2 (e.g., a solid form of Compound 2) comprises a step of cooling a mixture comprising Compound 2 to a suitable temperature (e.g., from about −20° C. to about 0° C.).

In some embodiments, Compound 2 (e.g., a solid form of Compound 2) precipitates from a mixture (e.g., a solution, suspension, or slurry). In some embodiments, Compound 2 crystallizes from a solution. In some embodiments, Compound 2 crystallizes from a solution following seeding of the solution (e.g., adding crystals of Compound 2 to the solution). In some embodiments, Compound 2 precipitates or crystallizes from a mixture after cooling, addition of an anti-solvent, and/or removal of all or part of a solvent through methods such as evaporation, distillation, filtration, reverse osmosis, absorption, or reaction.

In some embodiments, a method of preparing Compound 2 (e.g., a solid form of Compound 2) comprises a step of isolating Compound 2. It will be appreciated that Compound 2 may be isolated by any suitable means. In some embodiments, Compound 2 (e.g., a solid form of Compound 2) is separated from a supernatant by filtration. In some embodiments, Compound 2 (e.g., a solid form of Compound 2) is separated from a supernatant by decanting.

In some embodiments, isolated Compound 2 (e.g., an isolated solid form of Compound 2) is dried (e.g., in air or under reduced pressure, optionally at elevated temperature).

In some embodiments, a solid form of Compound 2 is prepared by converting one solid form of Compound 2 into another solid form of Compound 2.

In some embodiments, a solid form of Compound 2 is prepared by a process comprising a step of combining Compound 1 (e.g., amorphous Compound 1) in a suitable solvent (e.g., isopropanol) with stirring at a suitable temperature (e.g., about 40° C.). In some embodiments, the process further comprises adding a first portion (e.g., about 0.5 equiv) of a suitable acid (e.g., fumaric acid). In some embodiments, the process further comprises adding seed crystals of Compound 2 (e.g., seed crystals of Compound 1 Fumarate Form E). In some embodiments, the process further comprises adding a second, third, and/or fourth portion (e.g., about 0.2-0.3 equiv) of a suitable acid (e.g., fumaric acid). In some embodiments, the process further comprises adding a suitable anti-solvent (e.g., heptane). In some embodiments, the process further comprises cooling the mixture to ambient temperature (e.g., about 25° C.). In some embodiments, the process further comprises isolating a solid form of Compound 2 (e.g., Compound 1 Fumarate Form E) by a method such as filtration.

### Compositions

In some embodiments, the present disclosure also provides compositions comprising one or more solid and/or salt forms of Compound 1. In some embodiments, provided compositions comprise Compound 1, e.g., Compound 1 Form A Acetonitrile Solvate, Compound 1 Form A Acetone Solvate, Compound 1 Form A Tetrahydrofuran Solvate, Compound 1 Form A Dioxane Solvate, Compound 1 Form A Ethyl Acetate Solvate, Compound 1 Form A N-Methylpyrrolidone Solvate, Compound 1 Form B, or amorphous Compound 1, or a mixture thereof. In some embodiments, provided compositions comprise Compound 2, e.g., Compound 1 Malate Form A, Compound 1 Fumarate Form A Anhydrate, Compound 1 Fumarate Form A Ethyl Acetate Solvate, Compound 1 Fumarate Form C, Compound 1 Fumarate Form D, Compound 1 Fumarate Form E, Compound 1 Fumarate Form F, Compound 1 Fumarate Form G, Compound 1 Fumarate Form J, Compound 1 Oxalate Form A, Compound 1 Phosphate Form A, or Compound 1 Phosphate Form B, or a mixture thereof.

In some embodiments, a provided composition comprising a crystalline solid form (e.g., a crystalline solid form of Compound 1 and/or Compound 2) is substantially free of impurities. As used herein, the term “substantially free of impurities” means that the composition contains no significant amount of extraneous matter. Such extraneous matter may include starting materials, residual solvents, or any other impurities that may result from the preparation of and/or isolation of a crystalline solid form. In some embodiments, the composition comprises at least about 90% by weight of a crystalline solid form. In some embodiments, the composition comprises at least about 95% by weight of a crystalline solid form. In some embodiments, the composition comprises at least about 99% by weight of a crystalline solid form.

In some embodiments, a provided composition comprising a crystalline solid form (e.g., a crystalline solid form of Compound 1 or Compound 2) is substantially pure (e.g., comprises at least about 95%, 97%, 97.5%, 98%, 98.5%, 99%, 99.5%, or 99.8% by weight of the crystalline solid form based on the total weight of the composition). In some embodiments, a composition comprising a crystalline solid form (e.g., a crystalline solid form of Compound 1 or Compound 2) comprises no more than about 5.0 percent of total organic impurities. In some embodiments, a composition comprising a crystalline solid form (e.g., a crystalline solid form of Compound 1 or Compound 2) comprises no more than about 3.0 percent of total organic impurities. In some embodiments, a composition comprising a crystalline solid form (e.g., a crystalline solid form of Compound 1 or Compound 2) comprises no more than about 1.5 percent of total organic impurities. In some embodiments, a composition comprising a crystalline solid form (e.g., a crystalline solid form of Compound 1 or Compound 2) comprises no more than about 1.0 percent of total organic impurities. In some embodiments, a composition comprising a crystalline solid form (e.g., a crystalline solid form of Compound 1 or Compound 2) comprises no more than about 0.5 percent of total organic impurities. In some embodiments, the percent of total organic impurities is measured by HPLC.

In some embodiments, a composition comprises a crystalline solid form (e.g., a crystalline solid form of Compound 1 and/or Compound 2) and an amorphous solid form (e.g., an amorphous solid form of Compound 1 and/or Compound 2). In some embodiments, a composition comprising a crystalline solid form is substantially free of an amorphous solid form. As used herein, the term “substantially free of an amorphous solid form” means that the composition contains no significant amount of an amorphous solid form. In some embodiments, the composition comprises at least about 90% by weight of a crystalline solid form (e.g., a crystalline solid form of Compound 1 and/or Compound 2). In some embodiments, the composition comprises at least about 95% by weight of a crystalline solid form (e.g., a crystalline solid form of Compound 1 and/or Compound 2). In some embodiments, the composition comprises at least about 99% by weight of a crystalline solid form (e.g., a crystalline solid form of Compound 1 and/or Compound 2). In some embodiments, the composition comprises no more than about 10% by weight of an amorphous solid form (e.g., an amorphous solid form of Compound 1 and/or Compound 2). In some embodiments, the composition comprises no more than about 5% by weight of an amorphous solid form (e.g., a crystalline solid form of Compound 1 and/or Compound 2). In some embodiments, the composition comprises no more than about 1% by weight of an amorphous solid form (e.g., a crystalline solid form of Compound 1 and/or Compound 2).

In some embodiments, a composition comprises a free base form (e.g., Compound 1) and a salt form (e.g., Compound 2). In some such embodiments, a free base form is crystalline, amorphous, or a mixture thereof; in some such embodiments, a salt form is crystalline, amorphous, or a mixture thereof.

In some embodiments, a composition comprises a mixture of crystalline solid forms (e.g., a mixture of one or more crystalline forms of Compound 1 and/or Compound 2).

### Pharmaceutical Compositions

In some embodiments, the present disclosure provides a pharmaceutical composition comprising Compound 1, or a crystalline form and/or complex form thereof, and a pharmaceutically acceptable carrier. In some embodiments, the present disclosure provides a pharmaceutical composition comprising a solid form of Compound 1 (e.g., a solid form described herein) and a pharmaceutically acceptable carrier. In some embodiments, the present disclosure provides a pharmaceutical composition comprising Compound 2 (e.g., a solid form of Compound 2, e.g., a solid form described herein) and a pharmaceutically acceptable carrier.

In some embodiments, provided pharmaceutical compositions comprise an amount of Compound 1 (i.e., in any suitable form such as a crystalline and/or complex form) that is effective to measurably inhibit estrogen receptor (ER) or a mutant thereof in a biological sample or patient. In some embodiments, provided pharmaceutical compositions are formulated for oral administration.

In some embodiments, provided pharmaceutical compositions comprise Compound 1 (i.e., in any suitable form such as a crystalline and/or complex form) and one or more fillers, disintegrants, lubricants, glidants, anti-adherents, and/or anti-statics, etc.

Pharmaceutical compositions of the present disclosure may be administered orally, parenterally, by inhalation spray, topically, rectally, nasally, buccally, vaginally, intraperitoneally, intracisternally or via an implanted reservoir. In some embodiments, provided pharmaceutical compositions are administered orally, intraperitoneally or intravenously. In some embodiments, provided pharmaceutical compositions are administered orally.

In some embodiments, a provided pharmaceutical composition is an oral dosage form (e.g., a capsule or a tablet). In some embodiments, a provided pharmaceutical composition is a tablet. In some embodiments, a provided pharmaceutical composition is a capsule.

In some embodiments, a provided pharmaceutical composition is a solid pharmaceutical composition (e.g., a solid dosage form such as a capsule or tablet).

In some embodiments, a provided pharmaceutical composition comprises an amount of Compound 1 suitable to provide a human with a dose of Compound 1 that corresponds to 3, 4, 5, 6, 7, 8, 9, 10, 11, 12, 13, 14, 15, 16, 17, 18, 19, 20, 21, 22, 23, 24, 25, 26, 27, 28, 29, or 30 mg/kg in a mouse. In some embodiments, a provided pharmaceutical composition comprises an amount of Compound 1 suitable to provide a human with a dose of Compound 1 that corresponds to at least 3 mg/kg in a mouse. In some embodiments, a provided pharmaceutical composition comprises an amount of Compound 1 suitable to provide a human with a dose of Compound 1 that corresponds to at least 5 mg/kg in a mouse. In some embodiments, a provided pharmaceutical composition comprises an amount of Compound 1 suitable to provide a human with a dose of Compound 1 that corresponds to at least 10 mg/kg in a mouse. In some embodiments, a provided pharmaceutical composition comprises an amount of Compound 1 suitable to provide a human with a dose of Compound 1 that corresponds to at least 15 mg/kg in a mouse. In some embodiments, a provided pharmaceutical composition comprises an amount of Compound 1 suitable to provide a human with a dose of Compound 1 that corresponds to at least 20 mg/kg in a mouse. In some embodiments, a provided pharmaceutical composition comprises an amount of Compound 1 suitable to provide a human with a dose of Compound 1 that corresponds to at least 25 mg/kg in a mouse. In some embodiments, a provided pharmaceutical composition comprises an amount of Compound 1 suitable to provide a human with a dose of Compound 1 that corresponds to at least 30 mg/kg in a mouse.

In some embodiments, a provided pharmaceutical composition is administered once daily (QD). In some embodiments, a provided pharmaceutical composition is administered twice daily (BID). In some embodiments, a provided pharmaceutical composition is administered every other day (QOD). In some embodiments, a provided pharmaceutical composition is administered once weekly (QW). In some embodiments, a provided pharmaceutical composition is administered once every four weeks (Q4W).

In some embodiments, a provided pharmaceutical composition (e.g., a unit dosage form) comprises about 15 mg to about 120 mg of Compound 1. In some embodiments, a provided pharmaceutical composition (e.g., a unit dosage form) comprises about 15 mg to about 100 mg of Compound 1. In some embodiments, a provided pharmaceutical composition (e.g., a unit dosage form) comprises about 60 mg to about 120 mg of Compound 1. In some embodiments, a provided pharmaceutical composition (e.g., a unit dosage form) comprises about 15 mg, about 20 mg, about 30 mg, about 40 mg, about 50 mg, about 60 mg, about 70 mg, about 80 mg, about 90 mg, or about 100 mg of Compound 1. In some embodiments, a provided pharmaceutical composition (e.g., a unit dosage form) comprises about 15 mg of Compound 1. In some embodiments, a provided pharmaceutical composition (e.g., a unit dosage form) comprises about 30 mg of Compound 1. In some embodiments, a provided pharmaceutical composition (e.g., a unit dosage form) comprises about 60 mg of Compound 1. In some embodiments, a provided pharmaceutical composition (e.g., a unit dosage form) comprises about 90 mg of Compound 1. In some embodiments, a provided pharmaceutical composition (e.g., a unit dosage form) comprises about 120 mg of Compound 1. In some embodiments, a unit dosage form is a capsule. In some embodiments, a unit dosage form is a tablet.

It will be appreciated that reference to an amount (e.g., in mg) of Compound 1 in relation to, e.g., a pharmaceutical composition, dosing regimen, etc., means the weight amount that corresponds to Compound 1 in free base form. A person of skill in the art will appreciate how to use a free base weight of Compound 1 in a provided composition to determine a weight amount for a particular salt form (e.g., Compound 2) described herein. Accordingly, Compound 1 may be provided and/or utilized as, e.g., a salt form, such that the amount of the salt (or other form) is an amount that corresponds to the “free base equivalent” of Compound 1.

In some embodiments, a provided pharmaceutical composition is prepared by (i) providing Compound 1 in any suitable form such as a crystalline and/or complex form; and (ii) formulating the Compound 1 with suitable excipients, to provide the pharmaceutical composition.

### Uses

Compounds and compositions described herein are generally useful for the inhibition of the estrogen receptor (ER) and mutants thereof. In some embodiments, the present disclosure encompasses the insight that compounds and compositions described herein are useful for treatment of an ER-associated disorder (e.g., an ER-associated cancer, such as breast cancer, including metastatic brain cancer), detection of the same, and/or characterization of certain tumors.

For example, in some embodiments, the present disclosure provides certain methods of treatment in a subject having an ER-associated disease, disorder, or condition. In some embodiments, an ER-associated disease, disorder or condition is a cancer. In some embodiments, an ER-associated disease, disorder or condition is selected from breast cancer, bone cancer, lung cancer, colorectal cancer, endometrial cancer, prostate cancer, ovarian cancer, vaginal cancer, endometriosis, and uterine cancer. In some embodiments, an ER-associated disease, disorder, or condition is breast cancer.

In some embodiments, a subject has been determined or is suspected of having a cancer that has metastasized (e.g., to the brain, bones, lungs, liver, or the central nervous system). In some embodiments, a subject has been determined or is suspected of having brain metastases. In some embodiments, the subject has developed brain metastases related to an ER-associated cancer, e.g., breast cancer, or a mutation to the estrogen receptor.

In some embodiments, a provided method comprises administering Compound 1, or a crystalline form or complex form thereof, to a subject previously treated with an ER inhibitor. In some such embodiments, a provided method comprises administering Compound 1, or a crystalline form or complex form thereof, to a subject previously treated with a Selective Estrogen Receptor Modulator (SERM), including, for example, tamoxifen, endoxifene, raloxifene, toremifene, lasofoxifene, and ospemifene.

In some embodiments, a provided method comprises administering Compound 1, or a crystalline fonn or complex form thereof, to a subject suffering from an ER-associated disorder (e.g., breast cancer) that is unresponsive to therapy with a SERM, including, for example, tamoxifen, endoxifene, raloxifene, toremifene, lasofoxifene, and ospemifene.

In some embodiments, a subject has relapsed during or following therapy with a SERM, including, for example, tamoxifen, endoxifene, raloxifene, toremifene, lasofoxifene, and ospemifene.

In some embodiments, a provided method comprises administering Compound 1, or a crystalline form or complex form thereof, to a subject with estrogen receptor positive (ER+) and human epidermal growth factor receptor negative (HER−) disease. In some embodiments, a provided method comprises administering Compound 1, or a crystalline form or complex form thereof, to a subject with estrogen receptor positive (ER+) and human epidermal growth factor receptor positive (HER+) disease.

In some embodiments, Compound 1 is administered to the subject in an amount that is from about to 15 mg to about 360 mg. In some embodiments, Compound 1 is administered to the subject in an amount that is from about to 30 mg to about 360 mg. In some embodiments, Compound 1 is administered to the subject in an amount that is from about to 30 mg to about 300 mg. In some embodiments, Compound 1 is administered to the subject in an amount that is from about to 60 mg to about 120 mg. In some embodiments, Compound 1 is administered to the subject in an amount that is from about 15 mg to about 100 mg. In some embodiments, Compound 1 is administered to the subject in an amount that is about 15 mg, about 20 mg, about 30 mg, about 40 mg, about 50 mg, about 60 mg, about 70 mg, about 80 mg, about 90 mg, or about 100 mg. In some embodiments, Compound 1 is administered to the subject in an amount that is about 120 mg, about 150 mg, about 210 mg, or about 300 mg. In some embodiments, Compound 1 is administered to the subject in an amount that is about 30 mg. In some embodiments, Compound 1 is administered to the subject in an amount that is about 60 mg. In some embodiments, Compound 1 is administered to the subject in an amount that is about 90 mg. In some embodiments, Compound 1 is administered to the subject in an amount that is about 120 mg.

In some embodiments, Compound 1 is administered to the subject in an amount that is about 15 mg to about 360 mg per day (QD). In some embodiments, Compound 1 is administered to the subject in an amount that is about 30 mg to about 360 mg per day (QD). In some embodiments, Compound 1 is administered to the subject in an amount that is about 30 mg to about 300 mg per day (QD). In some embodiments, Compound 1 is administered to the subject in an amount that is about 60 mg to about 120 mg per day (QD). In some embodiments, Compound 1 is administered to the subject in an amount that is from about 15 mg to about 100 mg QD. In some embodiments, Compound 1 is administered to the subject in an amount that is about 15 mg, about 20 mg, about 30 mg, about 40 mg, about 50 mg, about 60 mg, about 70 mg, about 80 mg, about 90 mg, or about 100 mg QD. In some embodiments, Compound 1 is administered to the subject in an amount that is about 120 mg, about 150 mg, about 210 mg, or about 300 mg QD. In some embodiments, Compound 1 is administered to the subject in an amount that is about 30 mg QD. In some embodiments, Compound 1 is administered to the subject in an amount that is about 60 mg QD. In some embodiments, Compound 1 is administered to the subject in an amount that is about 90 mg QD. In some embodiments, Compound 1 is administered to the subject in an amount that is about 120 mg QD.

In some embodiments, Compound 1 is administered to the subject in a unit dosage form. In some embodiments, unit dosage form is a capsule or tablet. In some embodiments, a unit dosage form comprises about 15 mg to about 120 mg of Compound 1. In some embodiments, a unit dosage form comprises about 15 mg to about 100 mg of Compound 1. In some embodiments, a unit dosage form comprises about 60 mg to about 120 mg of Compound 1. In some embodiments, a unit dosage form comprises about 15 mg, about 20 mg, about 30 mg, about 40 mg, about 50 mg, about 60 mg, about 70 mg, about 80 mg, about 90 mg, or about 100 mg of Compound 1. In some embodiments, a unit dosage form comprises about 15 mg of Compound 1. In some embodiments, a unit dosage form comprises about 30 mg of Compound 1. In some embodiments, a unit dosage form comprises about 60 mg of Compound 1. In some embodiments, a unit dosage form comprises about 90 mg of Compound 1. In some embodiments, a unit dosage form comprises about 120 mg of Compound 1. In some embodiments, a unit dosage form is a capsule. In some embodiments, a unit dosage form is a tablet.

In some embodiments, a total daily dose of Compound 1 administered to the subject is in an amount that is about 15 mg to about 360 mg per day (QD). In some embodiments, a total daily dose of Compound 1 administered to the subject is about 30 mg to about 360 mg. In some embodiments, a total daily dose of Compound 1 administered to the subject is about 30 mg to about 300 mg. In some embodiments, a total daily dose of Compound 1 administered to the subject is about 60 mg to about 120 mg. In some embodiments, a total daily dose of Compound 1 administered to the subject is in an amount that is from about 15 mg to about 100 mg QD. In some embodiments, a total daily dose of Compound 1 administered to the subject is in an amount that is about 15 mg, about 20 mg, about 30 mg, about 40 mg, about 50 mg, about 60 mg, about 70 mg, about 80 mg, about 90 mg, or about 100 mg QD. In some embodiments, a total daily dose of Compound 1 administered to the subject is about 120 mg, about 150 mg, about 210 mg, or about 300 mg. In some embodiments, a total daily dose of Compound 1 administered to the subject is in an amount that is about 30 mg QD. In some embodiments, a total daily dose of Compound 1 administered to the subject is about 60 mg. In some embodiments, a total daily dose of Compound 1 administered to the subject is about 90 mg. In some embodiments, a total daily dose of Compound 1 administered to the subject is about 120 mg.

### Combination Therapy

The present disclosure encompasses the recognition that a combination of certain agents can beneficially be used to completely antagonize the estrogen receptor. Accordingly, in some embodiments, the present disclosure provides a method of treating a subject suffering from an ER-associated disorder (e.g., a cancer, e.g., a breast cancer) comprising administering a complete estrogen receptor antagonist and an anti-cancer agent. For example, in some embodiments, a complete estrogen receptor antagonist is Compound 1 in any suitable form (e.g., a crystalline form or complex form thereof). In some embodiments, an anti-cancer agent is a CDK 4/6 inhibitor, a PI3KCA inhibitor, or an mTOR inhibitor.

In some embodiments, the present disclosure provides a method of treating a patient or subject suffering from a cancer, the method comprising administering a complete estrogen receptor antagonist (e.g., Compound 1 in any suitable form) and a CDK4/6 inhibitor (i.e., an agent that inhibits one or both of CDK4 and CDK6). In some embodiments, an anti-cancer agent is a CDK4/6 inhibitor selected from palbociclib, ribociclib, abemaciclib, lerociclib, trilaciclib, and SHR6390. In some embodiments, a CDK4/6 inhibitor is palbocociclib. In some embodiments, a CDK4/6 inhibitor is ribociclib. In some embodiments, a CDK4/6 inhibitor is abemaciclib. In some embodiments, a CDK4/6 inhibitor is lerociclib. In some embodiments, a CDK4/6 inhibitor is trilaciclib. In some embodiments, a CDK 4/6 inhibitor is SHR6390.

In some embodiments, the present disclosure provides a method of treating a patient or subject suffering from a cancer, the method comprising administering a complete estrogen receptor antagonist (e.g., Compound 1 in any suitable form) and a PIK3CA inhibitor. In some embodiments, a PIK3CA inhibitor is selected from alpelisib, taselisib, and LY3023414. In some embodiments, a PIK3CA inhibitor is alpelisib. In some embodiments, a PIK3CA inhibitor is taselisib. In some embodiments, a PIK3CA inhibitor is LY3023414.

In some embodiments, the present disclosure provides a method of treating a patient or subject suffering from a cancer, the method comprising administering a complete estrogen receptor antagonist (e.g., Compound 1 in any suitable form) and an mTOR inhibitor. In some embodiments, an mTOR inhibitor is selected from sirolimus, temsirolimus, everolimus, and LY3023414. In some embodiments, an mTOR inhibitor is sirolimus. In some embodiments, an mTOR inhibitor is temsirolimus. In some embodiments, an mTOR inhibitor is everolimus. In some embodiments, an mTOR inhibitor is LY3023414.

In some embodiments, the present disclosure provides methods of treating a subject with ER+ and HER+disease with a complete estrogen receptor antagonist (e.g., Compound 1 in any suitable form) and a HER2 inhibitor. In some embodiments, a HER2 inhibitor is selected from tucatinib, pertuzumab, lapatinib, trastuzumab, ado-trastuzumab emtansine, trastuzumab deruxtecan, and neratinib.

It is understood that combination therapy comprising a complete estrogen receptor antagonist and an anti-cancer agent described herein can comprise administration of the agents simultaneously or separately. For example, in some embodiments, a complete estrogen receptor antagonist and an anti-cancer agent are administered simultaneously. In some embodiments, an anti-cancer agent is administered prior to administration of a complete estrogen receptor antagonist. In some embodiments, an anti-cancer agent is administered after administration of a complete estrogen receptor antagonist.

### EXAMPLES

The Examples provided herein document and support certain aspects of the present disclosure but are not intended to limit the scope of any claim. The following non-limiting examples are provided to further illustrate certain teachings provided by the present disclosure. Those of skill in the art, in light of the present application, will appreciate that various changes can be made in the specific embodiments that are illustrated in the present Examples without departing from the spirit and scope of the present teachings.

The following abbreviations may be used in the Examples below: aq. (aqueous); ACN (acetonitrile); CSA (camphorsulfonic acid); d (day or days); DCM (dichloromethane); DEA (diethylamine); DHP (dihydropyran); DMF (N,N-dimethylformamide); DIPEA (N,N-diisopropylethylamine); DMAP (4-dimethylaminopyridine); DMSO (dimethyl sulfoxide); EA (ethyl acetate); ee (enantiomeric excess); equiv. (equivalent); ethanol (EtOH); h (hour or hours); Hex (hexanes); HPLC (high-performance liquid chromatography); IPA (isopropyl alcohol); KHMDS (potassium bis(trimethylsilyl)amide); LAH (lithium aluminum hydride); LCMS (liquid chromatography-mass spectrometry); LDA (lithium diisopropylamide); LiHMDS (lithium bis(trimethylsilyl)amide); MeOH (methanol); min (minute or minutes); NMR (nuclear magnetic resonance); Pd/C (palladium on carbon); PPh₃0 (triphenylphosphine oxide); Pt/C (platinum on carbon); rb (round-bottomed); Rf (retention factor); rt or RT (room temperature); SM (starting material); TEA (triethylamine); THF (tetrahydrofuran); THP (tetrahydropyran); TLC (thin layer chromatography); TsOH (p-toluenesulfonic acid or tosylic acid); and UV (ultraviolet).

### Materials & Methods

### X-Ray Powder Diffraction (XRPD)

XRPD was performed with a Panalytical X′Pert³ Powder XRPD on a Si zero-background holder. The 20 position was calibrated against a Panalytical Si reference standard disc. The parameters used are provided below:

| Parameters | Reflection Mode |
|---|---|
| X-Ray wavelength | Cu, kα |
| | Kα1 (Å): 1.540598, Kα2 (Å): 1.544426 |
| | Kα2/Kα1 intensity ratio: 0.50 |
| X-Ray tube setting | 45 kV, 40 mA |
| Divergence slit | Fixed 1/8° |
| Scan mode | Continuous |
| Scan range (°2θ) | 3-40 |
| Scan step time [s] | 18.87 |
| Step size (°2θ) | 0.0131 |
| Test Time | 4 min 15 s |

Alternatively, XRPD was performed with PANalytical X′Pert PRO MPD or Empyrean diffractometers using an incident beam of Cu radiation produced using an Optix long, fine-focus source. An elliptically graded multilayer mirror was used to focus Cu Kα X-rays through the specimen and onto the detector. Prior to the analysis, a silicon specimen (NIST SRM 640f) was analyzed to verify the observed position of the Si 111 peak was consistent with the NIST-certified position. A specimen of the sample was sandwiched between Kapton films and analyzed in transmission geometry. A beam-stop, short antiscatter extension, and an antiscatter knife edge were used to minimize the background generated by air. Soller slits for the incident and diffracted beams were used to minimize broadening from axial divergence. Diffraction patterns were collected using a scanning position-sensitive detector (X′Celerator) located 240 mm from the specimen and Data Collector software v. 5.5.

A further alternative method comprised using a Bruker D8 Focus X-ray diffractomcter equipped with LynxEye detector. Samples were scanned from 3° to 420 (20), at a step size of 0.020 (20). The tube voltage and current were 40 KV and 40 mA, respectively.

### Single Crystal X-Ray Crystallography

X-ray intensity data were measured at 173.0 K (controlled by Oxford Cryostream 800) on a Bruker Venture X-ray diffractometer. Incoatec Microfocus Source (IS 3.0) monochromated Cu Kα radiation (λ=1.54178 Å, voltage=50 kV, current=1.1 mA) was used as the X-ray source. The intensity data was collected by a Photon II detector. The data collection strategy was optimized by the Bruker Apex3 software for a 0.83 Å resolution.

### Thermogravimetric Analysis (TGA) and Differential Scanning Calorimetry (DSC)

TGA data were collected using a TA Discovery 550 TGA from TA Instrument. DSC was performed using a TA Q2000 DSC from TA Instrument. DSC was calibrated with Indium reference standard, and the TGA was calibrated using nickel reference standard. Detailed parameters used are listed below:

| Parameters | TGA | DSC |
|---|---|---|
| Method | Ramp | Ramp |
| Sample pan | Platinum, open | Aluminum, crimped |

| | |
|---|---|
| Temperature | RT-380° C. |
| Heating rate | 10° C./min |
| Purge gas | N₂ |

Alternatively, TGA/DSC analyses were performed using a Mettler-Toledo TGA/DSC3+analyzer. Temperature and enthalpy adjustments were performed using indium, tin, zinc, aluminum, gold, and phenyl salicylate, and then verified with indium. The balance was verified with calcium oxalate. The samples were placed in an open aluminum pan, hermetically sealed, the lid pierced, and then inserted into the TG furnace. A weighed aluminum pan configured as the sample pan was placed on the reference platform. The furnace was heated under nitrogen.

A further alternative method comprise using a TGA Q500 (TA Instruments, US). About 1-5 mg of sample was placed in an open tarred aluminum pan, automatically weighed, and inserted into the TGA furnace. The sample was heated at a rate of 10° C./min to the final temperature (about 300° C.). DSC characterization was conducted on a DSC 250 (TA Instruments, US). About 1-5 mg of sample was placed into a DSC pinhole pan. The sample was heated at a rate of 10° C./min to the final temperature (about 300° C.). The change of heat flux with temperature was recorded.

### Proton Nuclear Magnetic Resonance (¹H NMR)

Solution NMR was collected on Bruker 500M NMR Spectrometer using DMSO-d₆ as solvents.

Alternatively, solution ¹H NMR spectra were acquired with an Avance 600 MHz NMR Spectrometer using deuterated DMSO.

### Dynamic Vapor Sorption (DVS)

Dynamic Vapor Sorption (DVS) was measured by a SMS (Surface Measurement Systems) DVS Intrinsic. Parameters for DVS test are listed below:

| Parameters | Values | |
|---|---|---|
| Temperature | 25° | C. |
| Sample size | 10-20 | mg |
| Gas and flow rate | N₂, 200 mL/min | |
| dm/dt | 0.002%/min | |
| Min. dm/dt stability duration | 10 | min |
| Max. equilibrium time | 180 | min |

| | |
|---|---|
| RH range | 0% RH-95% RH-0% RH-95% RH, |
| | 0% RH-95% RH-0% RH-95% RH |
| RH step size | 10% |

Alternatively, DVS was performed using Intrinsic DVS (System Measurement System, UK). About 30-50 mg of sample was placed in a sample basked and hung in the measuring chamber. For an isotherm test, the chamber temperature is maintained by a water bath at a constant 25±1° C. The sample was tested at a targeted RH from 0 to 90% full cycle in step mode. The analysis was performed in 10% RH increments. Time duration at each RH was set as 60 min so that the sample could reach equilibrium with the chamber environment. Data were collected in 20 s increments.

### High Performance Liquid Chromatography (HPLC)

The HPLC method used for solubility measurements is summarized below:

| Mobile Phase A | 0.1% TFA in water | | |
|---|---|---|---|
| Mobile Phase B | 0.1% TFA in ACN | | |
| Column | XBridge 3.5 μm, 4.6 × 150 mm | | |
| Column Temperature | 40° C. | | |
| Injector Volume | 10 μL | | |
| Detector Wavelength | 254 nm and 280 nm | | |
| Flow Rate | 1 mL/min | | |
| Gradient | Time | % Mobile | % Mobile |
| Program | (min) | Phase A | Phase B |
| | 0 | 90 | 10 |
| | 15.0 | 50 | 50 |
| | 20.0 | 20 | 80 |
| | 22.0 | 20 | 80 |
| | 30.0 | 90 | 10 |

### Gas Chromatography (GC)

GC analysis was performed on GC8890 (Agilent, US), using helium gas as carrier gas and nitrogen gas as makeup gas with a FID detector. The sample was 10 mg/mL in dimethylacetamide. The vaporized sample was carried by the carrier gas (mobile phase) into the chromatographic column. The parameters are summarized below:

| | |
|---|---|
| Injector | Auto-sampler, fast plunger mode, direct |
| | injection |
| Carrier gas | Helium |
| Chromatographic column | DB-64, Agilent, 30 m × 0.53 mm x 3 mm |
| Column flow | 6.4 mL/min |
| Inlet | Inlet temperature is 250° C. |
| | Split ratio is 10:1 |
| | Constant pressure mode |
| Temp. Program | 40° C. for 5 min; ramp at 10° C./min |
| | to 220° C. |
| Detector | Detector temperature is 250° C. |
| | Used nitrogen as makeup gas |
| | H₂:Air:N₂ = 40:400:30 (mL/min) |
| Injection volume | 1 μL |
| Needle wash | Used diluents as needle washing solvent |

### Example 1: Synthesis of Compound 1

A complete synthesis of Compound 1 is provided in PCT Pub. No. WO 2017/059139, which is incorporated herein by reference and repeated below.

### Preparation of 4-((1-propylazetidin-3-yl)oxy)benzaldehyde

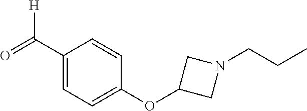 [SMILES: CCCN1CC(Oc2ccc(C=O)cc2)C1]

### Step 1: Preparation of 1-propionylazetidin-3-one

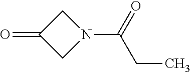

The compound 3-azetidinone hydrochloride (10.000 g, 93.0 mmol, 1.0 equiv.), anhydrous 1,2-dichloroethane (200 mL) and diisopropylethylamine (38.9 mL, 223 mmol, 2.4 equiv.) were added to a round bottom flask (500 mL) to provide a light yellow suspension. The suspension was sonicated for 1 h and then cooled to −10° C. (dry-ice/MeOH) for 10 min. Propionyl chloride (9.8 mL, 112 mmol, 1.2 equiv.) was added dropwise to the cooled suspension to provide an orange solution. The reaction was removed from the bath and stirred at room temperature for 16 h. The solvent was removed to provide a semi-solid. The semi-solid was suspended into EA (300 mL) and the suspension was filtered. The solid was rinsed with EA (2×100 mL). TLC analysis (10% MeOH/DCM, KMnO₇ stain/Heat) indicated there were three spots: Rf: 0.2, 0.5, 0.7. TLC (50% EA/Hex, KMnO₇ stain/Heat) indicated there were two spots: Rf: 1, 0.3. The filtrate was concentrated, adsorbed onto silica gel (25 g) and chromatographed through silica gel (100 g cartridge) with DCM (5 min) then 0-10% MeOH over 15 min. The product came off early from the column in DCM and continued to elute from the column with up to 10% MeOH. TLC in both solvent systems was carried out to determine if any propionyl chloride was present in early fractions. Fractions containing product were pooled and concentrated to afford the title compound as a yellow liquid (11.610 g, 98.2%).

¹H NMR (300 MHz, CDCl₃) δ: 4.80 (d, J=5.6 Hz, 4H), 2.29 (q, J=7.5 Hz, 2H), 2.01 (s, 3H), 1.18 (t, J=7.5 Hz, 3H).

### Step 2. Preparation of 1-propylazetidin-3-ol

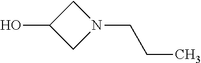

Lithium aluminum hydride (10.397 g, 273.9 mmol, 3.0 equiv.) was suspended into THF (200 mL) and cooled in an ice bath. A solution of 1-propionylazetidin-3-one (11.610 g, 91.3 mmol, 1.0 equiv.) in THF (100 mL) was added dropwise to the reaction mixture via a pressure equalizing addition funnel over 30 min. The addition funnel was removed. The flask was then fitted with a condenser and the reaction was heated at reflux in an oil bath at 75° C. for 16 h. The reaction was cooled in an ice bath for 20 min and sodium sulfate decahydrate (Glauber's salt, 25 g) was added in small portions over 20 min. After complete addition, the mixture was stirred at room temperature for 2 h. The mixture was filtered through a bed of Celite© (2 cm) and the solids rinsed with EA (2×250 mL). The clear solution was concentrated to a pale yellow liquid (9.580 g, 91.1%). NMR indicated the presence of THE and EA. This material was used without further purification in the preparation of the compounds of the examples below.

¹H NMR (300 MHz, CDCl₃) δ: 4.39 (pent, J=6 Hz, 1H), 3.62-3.56 (m, 2H), 2.90-2.85 (m, 2H), 2.41 (t, J=7.5 Hz, 2H), 1.34 (hextet, J=7.2 Hz, 2H), 0.87 (t, J=7.8 Hz, 3H).

### Step 3. Preparation of 4-((1-propylazetidin-3-yl)oxy)benzaldehyde

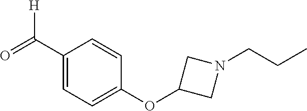 [SMILES: CCCN1CC(Oc2ccc(C=O)cc2)C1]

4-Fluorobenzaldehyde (15.00 g, 120.9 mmol, 0.9 equiv.), 1-propylazetidin-3-ol (15.00 g, 130.2 mmol, 1.0 equiv.), cesium carbonate (88.40 g, 271.3 mmol, 2.1 equiv.) and N,N-dimethylformamide (284 mL) were mixed together with a Teflon™ stir bar in a 500 mL round bottomed flask. The flask was sealed and heated in a heat block at 95° C. for 6 h. The reaction was analyzed by LCMS to indicate the aldehyde was consumed. The suspension was filtered through a sintered glass funnel and the solid was washed with ethyl acetate (100 mL). The filtrate was concentrated to an orange suspension. The suspension was mixed with water (200 mL) and ethyl acetate (200 mL) and the organic layer was washed with water (3×200 mL), brine, dried over anhydrous magnesium sulfate, filtered and concentrated to an orange liquid (21.74 g, 76.1%). The material was used without further purification.

¹HNMR (300 MHz, CDCl₃), δ 9.87 (s, 1H), 7.82 (d, J=9.0 Hz, 2H), 6.86 (d, J=8.7 Hz, 2H), 4.86 (quintet, J=5.7 Hz, 1H), 3.85-3.80 (m, 2H), 3.13-3.08 (m, 2H), 2.48 (t, J=7.2 Hz, 2H), 1.46-1.34 (m, 2H), 0.91 (t, J=7.2 Hz, 3H).

### Preparation of (R)-1-(1H-indol-3-yl)-N-((R)-1-phenylethyl)propan-2-anmine

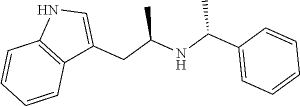 [SMILES: C[C@H](Cc1c[nH]c2ccccc12)N[C@H](C)c1ccccc1]

Indole-3-acetonc (25.0 g, 144 mmol, 1.0 equiv.) was added to a solution of (R)-(+)-1-phenylethylamine (23.0 mL, 181 mmol, 1.3 equiv.) in dichloromethane (600 mL) under N₂ at 25° C. and the mixture was allowed to stir for 1 hr. The reaction was cooled to 0-5° C. and sodium triacetoxyborohydride (100 g, 472 mmol, 3.3 equiv.) was added over 30 minutes via powder addition funnel to the ice cooled solution. The orange solution was stirred for 1 h at 0° C. and then was allowed to warm to RT. The reaction was stirred at RT for 19 h. At this time, ESI+indicated that no indole starting material was present. Saturated NaHCO₃ solution (100 mL) was added in 5 mL portions over 15 min at 10° C. with vigorous stirring. The solution was stirred for 15 min and sat. Na₂CO₃ solution (200 mL) was added over 15 minutes. Solid K₂CO₃ (9 g) was added in 3 g portions at which point the aqueous layer was pH 12 and bubbles had stopped forming. The layers were filtered and separated. The red organic layer was washed with sat. aq. NaHCO₃ (2×100 mL). The aqueous layers were combined and extracted with DCM (2×100 mL). The combined organic layers were dried over Na₂SO₄, filtered and concentrated to give the crude product (49 g). TLC (90:10 DCM:MeOH) showed four spots (Rf=0.63, 0.50, 0.16, 0.26), two of which were the separated diastereomeric major products (Rf=0.16 and 0.26). The crude was adsorbed onto silica gel and purified via flash chromatography (330 g cartridge, 0-100% EA:Hex). Fractions containing the R,R diastereomer were pooled and purified a second time with the same flash chromatography conditions to afford 24 g of product (˜82% ee). Previous successful separation was achieved by a silica gel:crude ratio of 40:1, so the mixture was divided into 3 portions and separated on 3×330 g silica gel cartridges (0-40% EA/Hex for 20 min, isocratic 40% EA/Hex 40 min). All fractions containing the desired product were >99% diastereomerically pure. Pure fractions were concentrated and pooled to yield (R)-1-(1H-indol-3-yl)-N-((R)-1-phenylethyl)-propan-2-amine as an orange semi-solid (11.91 g, 29.6%).

¹H NMR (CDCl₃, 300 MHz) R,R diastereomer: δ 0.96 (d, J=6.6 Hz, 3H), 1.30 (d, J=6.6 Hz, 3H), 2.68 (q, J=7.2 Hz, 1H), 2.97 (m, 2H) 4.00 (q, J=6.3 Hz, 1H), 7.43-6.97 (m, 10H), 7.96 (br s, 1H). R,S diastereomer: δ 1.11 (d, J=5.7 Hz, 3H), 1.30 (d, J=5.4 Hz, 3H) 2.80 (m, 3H), 3.92 (q, J=6.9 Hz, 1H), 6.93-7.40 (m, 10H), 8.13 (br s, 1H); the aromatic region was difficult to distinguish from the R,R diastereomer due to lack of purity.

LCMS: ES+[M+H]+ 279.0.

### Preparation of (2R)-]-(1H-indol-3-yl)propan-2-amine

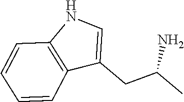

The compound (R)-1-(1H-indol-3-yl)-N-((R)-1-phenylethyl)propan-2-amine (11.91 g, 42.8 mmol, 1.0 equiv.) was dissolved in methanol (250 mL) and added to a 2 L Parr bottle and the solution was sparged with N₂ for 10 min. 20% Pd(OH)₂ on carbon wet with water (10.71 g, 76.3 mmol, 1.8 equiv.) was added and the bottle was pressurized with 50 psi of hydrogen and shaken in a Parr apparatus for 22 h, LCMS analysis indicated that the reaction was completed. The suspension was filtered through Celite® and concentrated to remove MeOH. The crude was dissolved into DCM and washed with saturated Na₂CO₃ solution (50 mL) and the aqueous layer was extracted with DCM (2×50 mL). The organic layers were combined, dried, and concentrated to yield (2R)-1-(1H-indol-3-yl)propan-2-amine as a light brown solid that did not require further purification (6.68 g, 89.6%).

¹H NMR (CDCl₃, 300 MHz) δ 1.17 (d, J=6.6 Hz, 3H), 2.66 (dd, J=8.4, 14.7 Hz, 1H), 2.88 (dd, J=5.4, 14.1 Hz, 1H), 3.27 (sextet, J=1.5 Hz, 1H), 7.05-7.22 (m, 3H), 7.37 (d, J=7.5 Hz, 1H), 7.62 (d, J=8.7 Hz, 1H), 8.00 (br s, 1H).

LCMS: ES+[M+H]+ 174.9.

### Preparation of 2-fluoro-2-methyipropanol

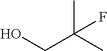

Methyl 2-fluoro-2-methylpropionate (5.01 g, 40.5 mmol, 1.0 equiv.) was added dropwise over 15 min to a stirred suspension of lithium aluminum hydride (2.50 g, 65.9 mmol, 1.6 equiv.) in anhydrous diethyl ether (100 mL) cooled in an ice bath. After 2 hours, 2.0 mL water, 2.0 mL 15% w/v NaOH, and 5.0 mL water were added sequentially dropwise. After 15 min, the white suspension was diluted with DCM, gravity filtered through Celite®, and the solids were washed with DCM. The filtrate was concentrated (200 mbar, 25° C.) to afford 2-fluoro-2-methylpropanol as a colorless oil (2.09 g, 56.1%).

¹H NMR (300 MHz, CDCl₃) δ 1.34 (d, J=21.3 Hz, 6H), 1.95 (br t, 1H), 3.56 (dd, J=6.6, 20.7 Hz, 2H).

### Preparation of 2-fluoro-2-methylpropyl trifluoromethanesulfonate

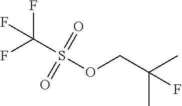

Trifluoromethanesulfonic anhydride (5.0 mL, 29.7 mmol, 1.3 equiv.) was added dropwise to a 0° C. solution of 2-fluoro-2-methylpropanol (2.090 g, 22.7 mmol, 1.0 equiv.) and 2,6-lutidine (3.40 mL, 29.4 mmol, 1.3 equiv.) in DCM (25 mL) over 30 minutes. After 2 hours, the red solution had turned light brown. TLC (20:80 EA:Hex, KMnO₄ stain) indicated that the starting material was not present. The reaction mixture was washed with 1M HCl solution (2×20 mL) and sat. NaHCO₃ solution (2×20 mL). The aqueous layers were each back extracted with DCM (20 mL). The combined organic layers were dried with Na₂SO₄, filtered and concentrated under reduced pressure (150 mbar, 25° C.) to afford 2-fluoro-2-methylpropyl trifluoromethanesulfonate as a red oil (4.39 g, 86.3%).

¹H NMR (300 MHz, CDCl₃) δ 1.46 (d, J=20.4 Hz, 6H), 4.41 (d, J=18.6 Hz, 2H). ¹⁹F NMR (282 MHz, CDCl₃) δ -147.1, −74.5.

### Preparation of (R)-N-(1-(1H-indol-3-yl)propan-2-yl)-2-fluoro-2-methylpropan-1-amine

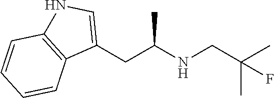 [SMILES: C[C@H](Cc1c[nH]c2ccccc12)NCC(C)(C)F]

The compound 2-fluoro-2-methylpropyl trifluoromethanesulfonate (9.587 g, 42.8 mmol, 1.1 equiv.) (solution in DCM, 16% DCM by wt %, 11.4384 g) was added to a solution of (2R)-1-(1H-indol-3-yl)propan-2-amine (6.680 g, 38.3 mmol, 1.0 equiv.), anhydrous 1,4-dioxanes (60.000 ml, 701.4 mmol, 18.3 equiv.), and freshly-distilled diisopropylethylamine (8.500 ml, 48.8 mmol, 1.3 equiv.). The dark brown solution was heated at 90° C. for 3 hours. After 3h, LCMS indicated that a small amount of indolamine starting material was still present. TLC (10% MeOH/DCM) indicated triflate (Rf=0.54) had been used up. NMR of unused triflate SM (286-30) indicated the triflate had not decomposed overnight, so another 0.1 equiv (0.9883 g, 13% DCM wt %, 0.8563 g triflate SM) was added and the reaction was heated for 2 h at 90° C. LCMS indicated the reaction had completed and TLC (10% MeOH/DCM) showed one spot (Rf=0.24) (TLC with 50% EA/Hex, 1 streaked spot Rf<=0.12, another spot at Rf=0). EtOAc (50 mL) was added and the solution was washed with NaHCO₃ (2×50 mL) and the combined aqueous layer was washed with EtOAc (50 mL). The combined organic extracts were dried over Na₂SO₄ and concentrated under reduced pressure. The crude (brown oil, 14.8 g) was purified via flash silica chromatography (240 g cartridge, 0-100% EA/Hex). The desired product eluted as a long tailing peak. Pure fractions were concentrated to yield (R)-N-(1-(1H-indol-3-yl)propan-2-yl)-2-fluoro-2-methylpropan-1-amine (4.211 g, 17.0 mmol) as a dark yellow oil.

¹H NMR (300 MHz, CDCl₃) δ 1.10 (d, J=6.3 Hz, 3H), 1.34 (dd, J=3.0, 21.9 Hz, 6H), 2.68-2.95 (m, 4H), 3.02 (sextet, J=6.6 Hz, 1H), 7.05 (d, J=2.4 Hz, 1H), 7.26-7.11 (m, 2H), 7.36 (d, J=6.9 Hz, 1H), 7.62 (d, J=7.5 Hz, 1H), 8.18 (br s, 1H). ¹⁹F NMR (282 MHz, CDCl₃) δ -144.2. m/z: ES+[M+H]+ 249.0.

### Preparation of Compound 1

4-((1-propylazetidin-3-yl)oxy)benzaldehyde (0.096 g, 0.4 mmol, 1.3 equiv.) was added to a solution of (R)-N-(1-(1H-indol-3-yl)propan-2-yl)-2-fluoro-2-methylpropan-1-amine (0.070 g, 0.3 mmol, 1.0 equiv.) in anhydrous toluene (1.50 mL) and glacial acetic acid (0.100 mL, 1.7 mmol, 6.2 equiv.). Molecular sieves were added and the solution was stirred under N₂ in the dark at 80° C. for 8 hours. The reaction solution was diluted in DCM, filtered, and washed with saturated Na₂CO₃ solution. The aqueous layer was extracted with DCM and the combined organic layers were dried over Na₂SO₄. The solution was filtered and concentrated. The residue was dissolved into acetonitrile (2 mL) and filtered through a syringe filter before purification via prep LC (40 to 90% ACN:H₂O over 18 min, followed by isocratic 90% ACN for 7 min). Pure fractions were concentrated and dried to afford (1R,3R)-2-(2-fluoro-2-methylpropyl)-3-methyl-1-(4-((1-propylazetidin-3-yl)oxy)phenyl)-2,3,4,9,-tetrahydro-1H-pyrido[3,4-b]indole as a white powder.

¹H NMR (300 MHz, CDCl₃) δ 0.90 (t, J=7.5 Hz, 3H), 1.09 (d, J=7.2 Hz, 3H), 1.26-1.50 (m, 8H), 2.45-2.77 (m, 6H), 3.01 (t, J=7.2 Hz, 2H), 3.34 (m, 1H), 3.77 (m, 2H), 4.60 (quin, J=5.7 Hz, 1H), 5.03 (s, 1H), 6.64 (d, J=8.1 Hz, 2H), 7.10-7.21 (m, 5H), 7.54 (d, J=7.5 Hz, 1H), 8.19 (br s, 1H). m/z: ES+[M+H]+ 450.2.

### Example 2: Preparation and Characterization of Compound 1 Solid Forms

### Compound 1 Form A Acetonitrile Solvate

Compound 1 Form A Acetonitrile Solvate was prepared according to the following exemplary procedure: About 20 mg of amorphous Compound 1 was suspended in 0.3 mL of acetonitrile at RT for 4 days. The remaining solids were isolated to give Compound 1 Form A Acetonitrile Solvate.

The XRPD pattern of Compound 1 Form A Acetonitrile Solvate is shown in FIG. 1 (top), and the corresponding data are summarized below:

Position ± 0.2 (degrees 2-Theta)

5.31
10.34
10.61
11.49
12.40
13.50
14.72
16.04
17.45
17.66
18.02
18.43
19.00
19.76
20.44
20.66
21.60
22.03
23.03
23.87
25.07
25.27
26.37
27.10
27.65
29.13
30.63

As shown by DSC curve in FIG. 2, the sample displayed one endothermic peak 82-87° C. (onset temperature). FIG. 2 also shows the TGA curve, which shows a weight loss of 5.57% up to 125° C.

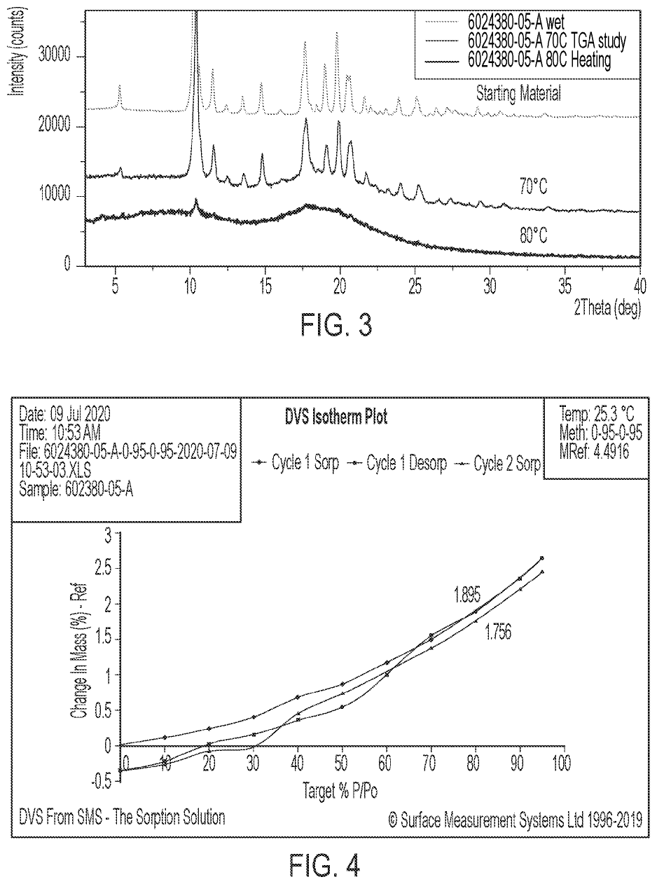

Upon heating Compound 1 Form A Acetonitrile Solvate to 70° C. and 80° C., it became amorphous (FIG. 3).

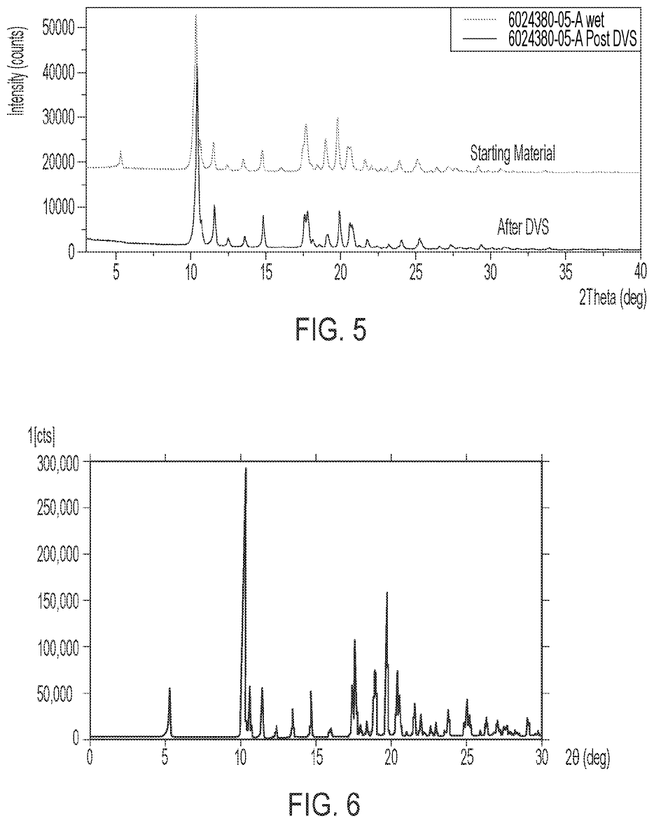

DVS of Compound 1 Form A Acetonitrile Solvate showed that was slightly hygroscopic (1.9% water uptake, FIG. 4) and exhibited the same crystalline pattern after DVS (FIG. 5).

Compound 1 Form A Acetonitrile Solvate was also prepared according to the following procedure: Approximately 2.34 g of amorphous Compound 1 was suspended in −50 mL acetonitrile at ambient temperature with stirring. A clear solution was initially produced, which was followed by precipitation. The resulting suspension was stirred at ambient temperature. After −3 days, the supernatant was pipetted out. Solids separated from the supernatant were transferred onto a paper filter and patted gently between paper folds to remove excess solvent. Secondary drying was not conducted. The material was composed of loose powder, which was consistent with Compound 1 Form A Acetonitrile Solvate by XRPD (FIG. 6). The TGA thermogram for the material exhibited a series of overlapped weight losses beginning at 31° C. A 3.6 wt % loss was observed between 31° C. and 81° C., followed by 1.7 wt % and 10.2 wt % losses between 81° C. and 96° C. and 96° C. and 175° C., respectively (FIG. 7). By DSC, the material displayed multiple broad endotherms with a major peak maximum of 76° C., followed by a noisy endotherm at 96° C. (onset) (FIG. 7).

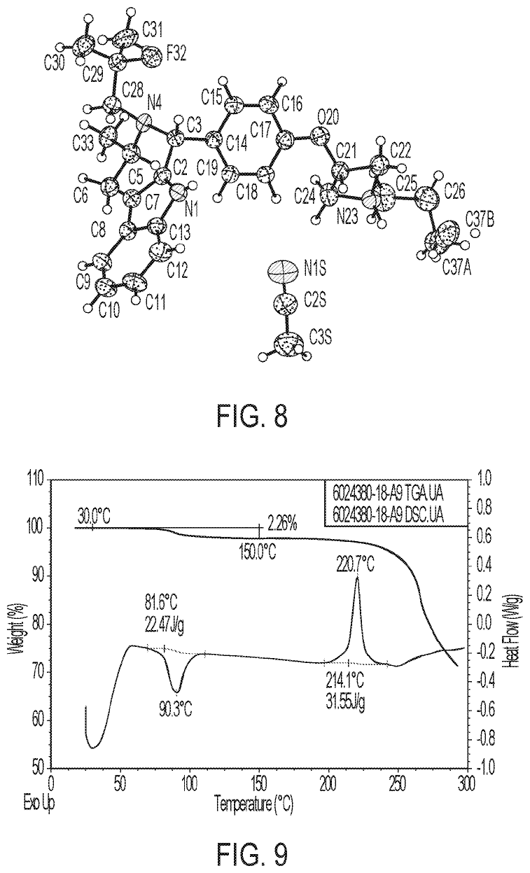

Single crystals of Compound 1 Form A Acetonitrile Solvate were grown serendipitously from acetonitrile. A Thermal Ellipsoid plot of the crystal structure is shown in FIG. 8. A plate-like single crystal with high diffraction quality, selected out from the batch, was immersed in MiTeGen LV5 (an oil based cryoprotectant) and mounted on a MiTeGen cryoloop in a random orientation and immersed in a stream of liquid nitrogen at 173K. The X-ray intensity data were measured on a Bruker D8 VENTURE (I S microfocus X-ray source, Cu Ku, X=1.54178 Å, PHOTON CMOS detector) diffractometer. The frames were integrated with the Bruker SAINT software package. The integration of the data using an trigonal unit cell in the space group P3₂21 yielded a total of 50168 reflections to a maximum θ angle of 66.631° (0.83 Å resolution), of which 5089 were independent (Rᵢₙₜ=9.38%) and were greater than 2σ(F²). The final cell constants of a=b=10.0115(2)Å, c=49.8608(16)Å, α=β=90°, γ=120°, cell volume=4328.0(2) Å³, are based upon the refinement of the XYZ-centroids of 4341 reflections above 10 σ(I) with 2.658°<θ<66.631°. Data were corrected for absorption effects using the Multi-Scan method (SADABS). The absorption coefficient μ of this material is 0.585 mm⁻¹ at this wavelength (1.54178 Å). The calculated minimum and maximum transmission coefficients (based on crystal size) are 0.929 and 0.984. The agreement factor for the structure (R₁) was 4.74% based on intensity. The single crystal parameters are summarized below:

| Empirical formula | C30 H40 F N4 O | |
|---|---|---|
| Formula weight | 491.66 | |
| Temperature | 173(2) K | |
| Wavelength | 1.54178 Å | |
| Crystal system | Trigonal | |
| Space group | P3₂21 | |
| Unit cell dimensions | a = 10.0115(2) Å | α = 90°. |
| | b = 10.0115(2) Å | β = 90°. |
| | c = 49.8608(16) Å | γ = 120°. |

| | |
|---|---|
| Volume | 4328.0(2) Å³ |
| Z | 6 |
| Density (calculated) | 1.132 Mg/m³ |
| Absorption coefficient | 0.585 mm⁻¹ |
| F(000) | 1590 |
| Crystal size | 0.128 × 0.111 × 0.028 mm³ |
| Theta range for data collection | 2.658 to 66.631°. |
| Index ranges | −11 <= h <= 11, −11 <= k <= |
| | 11, −59 <= 1 <= 59 |
| Reflections collected | 50168 |
| Independent reflections | 5089 [R(int) = 0.0938] |
| Completeness to theta = 66.631° | 100.0% |
| Refinement method | Full-matrix least-squares on F² |
| Data / restraints / parameters | 5089 / 0 / 347 |
| Goodness-of-fit on F² | 1.058 |
| Final R indices [I > 2sigma(I)] | R1 = 0.0474, wR2 = 0.1177 |
| R indices (all data) | R1 = 0.0574, wR2 = 0.1263 |
| Absolute structure parameter | 0.3(3) |
| Extinction coefficient | 0.0021(3) |
| Largest diff. peak and hole | 0.280 and −0.394e · Å⁻³ |

### Compound 1 Form A Acetone Solvate

Compound 1 Form A Acetone Solvate was prepared according to the following exemplary procedure: About 30 mg of amorphous Compound 1 was suspended in 0.3 mL of acetone/water (1:4) at 5° C. for 4 days. The remaining solids were isolated to give Compound 1 Form A Acetone Solvate.

The XRPD pattern of Compound 1 Form A Acetone Solvate is shown in FIG. 1 (middle), and the corresponding data are summarized below:

Position ± 0.2 (degrees 2-Theta)

4.72
5.31
10.33
11.51
13.54
14.72
17.47
17.69
18.47
19.05
19.80
20.49
21.69
23.94
25.13
27.22
29.21
33.71

As shown by DSC curve in FIG. 9, the sample displayed one endothermic peak 82-87° C. (onset temperature). FIG. 9 also shows the TGA curve, which shows a weight loss of 2.26% up to 150° C.

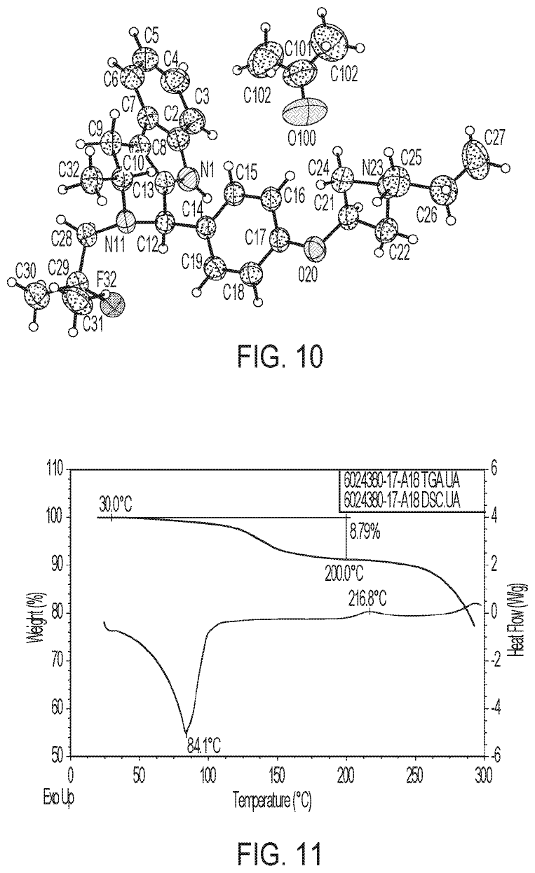

Single crystals of Compound 1 Form A Acetone Solvate were grown serendipitously from acetone. A Thermal Ellipsoid plot of the compound in the crystal is shown in FIG. 10. A rod-like single crystal with high diffraction quality, selected out from the batch, was immersed in MiTeGen LV5 (an oil based cryoprotectant) and mounted on a MiTeGen cryoloop in a random orientation and immersed in a stream of liquid nitrogen at 173K. The X-ray intensity data were measured on a Bruker D8 VENTURE (IVS microfocus X-ray source, Cu Kα, λ=1.54178 Å, PHOTON CMOS detector) diffractometer. The frames were integrated with the Bruker SAINT software package. The integration of the data using an trigonal unit cell in the space group P3₂21 yielded a total of 60744 reflections to a maximum θ angle of 66.533° (0.83 Å resolution), of which 5049 were independent (Rᵢₙₜ=12.68%) and were greater than 2σ(F²). The final cell constants of α=b=9.9835(6)Å, c=49.787(6)Å, α=β=90°, γ=120°, cell volume=2927.1(5) Å³, are based upon the refinement of the XYZ-centroids of 4820 reflections above 10 σ(I) with 2.663°<θ<64.885°. Data were corrected for absorption effects using the Multi-Scan method (SADABS). The absorption coefficient μ of this material is 0.579 mm⁻¹ at this wavelength (1.54178 Å). The calculated minimum and maximum transmission coefficients (based on crystal size) are 0.946 and 0.989. The agreement factor for the structure (R₁) was 5.91% based on intensity. The single crystal parameters are summarized below:

| Empirical formula | C59 H78 F2 N6 O3 | |
|---|---|---|
| Formula weight | 957.27 | |
| Temperature | 173(2) K | |
| Wavelength | 1.54178 Å | |
| Crystal system | Trigonal | |
| Space group | P3₂21 | |
| Unit cell dimensions | a = 9.9835(6) Å | α = 90°. |
| | b = 9.9835(6) Å | β = 90°. |
| | c = 49.787(4) Å | γ = 120°. |

| | |
|---|---|
| Volume | 4297.5(6) Å³ |
| Z | 3 |
| Density (calculated) | 1.110 Mg/m³ |
| Absorption coefficient | 0.579 mm⁻¹ |
| F(000) | 1548 |
| Crystal size | 0.096 × 0.056 × 0.020 mm³ |
| Theta range for data collection | 2.662 to 66.533°. |
| Index ranges | −11 <= h <= 11, −11 <= k <= |
| | 11, −58 <= 1 <= 56 |
| Reflections collected | 60744 |
| Independent reflections | 5049 [R(int) = 0.1268] |
| Completeness to theta = 66.533° | 99.9% |
| Absorption correction | Semi-empirical from equivalents |
| Max. and min. transmission | 0.7258 and 0.6622 |
| Refinement method | Full-matrix least-squares on H2 |
| Data / restraints / parameters | 5049 / 0 / 318 |
| Goodness-of-fit on F² | 1.224 |
| Final R indices [I > 2sigma(I)] | R1 = 0.0591, wR2 = 0.1377 |
| R indices (all data) | R1 = 0.0840, wR2 = 0.1526 |
| Absolute structure parameter | −0.17(15) |
| Extinction coefficient | 0.0053(5) |
| Largest diff. peak and hole | 0.279 and −0.213 e · Å⁻³ |

### Compound 1 Form A Tetrahydrofuran Solvate

Compound 1 Form A Tetrahydrofuran Solvate was prepared according to the following exemplary procedure: About 20 mg of amorphous Compound 1 was suspended in 0.3 mL of tetrahydrofuran/water (1:9) at RT for 4 days. The remaining solids were isolated to give Compound 1 Form A Tetrahydrofuran Solvate.

The XRPD pattern of Compound 1 Form A Tetrahydrofuran Solvate is shown in FIG. 1 (bottom), and the corresponding data are summarized below:

Position ± 0.2 (degrees 2-Theta)

5.29
10.16
10.31
10.57
11.47
13.52
14.69
15.86
17.81
18.42
18.99
19.75
20.40
20.73
21.57
22.31
23.04
23.83
23.95
25.04
27.12
27.72
29.12

As shown by DSC curve in FIG. 11, the sample displayed one endothermic peak 82-87° C. (onset temperature). FIG. 11 also shows the TGA curve, which shows a weight loss of 8.79% up to 200° C.

### Compound 1 Form A Dioxane Solvate

Compound 1 Form A Dioxane Solvate was prepared according to the following exemplary procedure: Approximately 30-80 mg amorphous Compound 1 was slurried in dioxane/water (60:40) at ambient temperature. After approximately 6 days, solids were isolated to give Compound 1 Form A Dioxane Solvate.

The XRPD pattern of Compound 1 Form A Dioxane Solvate is shown in FIG. 13, and the corresponding data are summarized below:

Position (degrees 2-Theta)

5.25
10.10
10.25
10.53
11.40
13.37
14.61
17.35
17.54
18.82
19.64
20.34
21.48
21.90

As shown by DSC curve in FIG. 14, the sample displayed an asymmetric broad endotherm at 126° C. (peak temperature). FIG. 14 also shows the TGA curve, which shows a weight loss of 8.7% between 72° C. and 221° C.

### Compound 1 Form A Ethyl Acetate Solvate

Compound 1 Form A Ethyl Acetate Solvate was prepared according to the following exemplary procedure: Initially, ˜30-80 mg amorphous Compound 1 was heated until liquefaction was observed. The liquefied sample was cooled to ambient temperature and exposed to dried EtOAc vapors, which, later resulted in dissolution due to the solvent migration. The solution was slowly evaporated and yielded crystals with birefringence and extinction. An attempt was made to separate selected crystals from the bulk sample for single crystal data collection. However, the crystals were observed to break during the isolation. The entire sample was then analyzed by XRPD.

The XRPD pattern of Compound 1 Form A Ethyl Acetate Solvate is shown in FIG. 15, and the corresponding data are summarized below:

Position (degrees 2-Theta)

5.25
10.10
10.25
10.53
11.40
13.37
14.61
17.35
17.54
18.82
19.64
20.34
21.48
21.90

### Compound 1 Form A N-Methylpyrrolidone Solvate

Compound 1 Form A N-Methylpyrrolidone Solvate was prepared according to the following exemplary procedure: Approximately 30-80 mg amorphous Compound 1 was slurried in NMP/water (60:40) at ambient temperature. After approximately 6 days, solids were isolated to give Compound 1 Form A N-Methylpyrrolidone Solvate.

The XRPD pattern of Compound 1 Form A N-Methylpyrrolidone Solvate is shown in FIG. 12.

As shown by DSC curve in FIG. 16, the sample displayed a broad endotherm at 95° C. (peak temperature). FIG. 16 also shows the TGA curve, which shows a weight loss of 9.0% between 89° C. and 239° C.

### Compound 1 Form B

Compound 1 Form B was prepared according to the following exemplary procedure: About 15 mg of amorphous Compound 1 was weighed into a 3-mL vial, which was placed into a 20-mL vial with 4 mL of DMSO. The 20-mL vial was sealed with a cap and kept at RT for 9 days allowing solvent vapor to interact with sample. The solids were collected to give Compound 1 Form B.

The XRPD pattern of Compound 1 Form B is shown in FIG. 17, and the corresponding data are summarized below:

Position (degrees 2-Theta)

7.09
8.45
8.85
10.28
11.73
12.14
13.07
13.37
13.78
14.19
15.10
15.67
16.07
16.58
16.96
17.71
18.14
18.62
18.85
19.43
19.73
20.16
20.67
21.34
21.61
22.85
23.57
24.11
24.69
24.99
26.30
26.65
28.31

As shown by DSC curve in FIG. 18, the sample displayed one endothermic peak at 74.0° C. (onset temperature). FIG. 18 also shows the TGA curve, which shows a weight loss of 14.98% up to 200° C. Compound 1 Form B was determined to be a DMSO solvate.

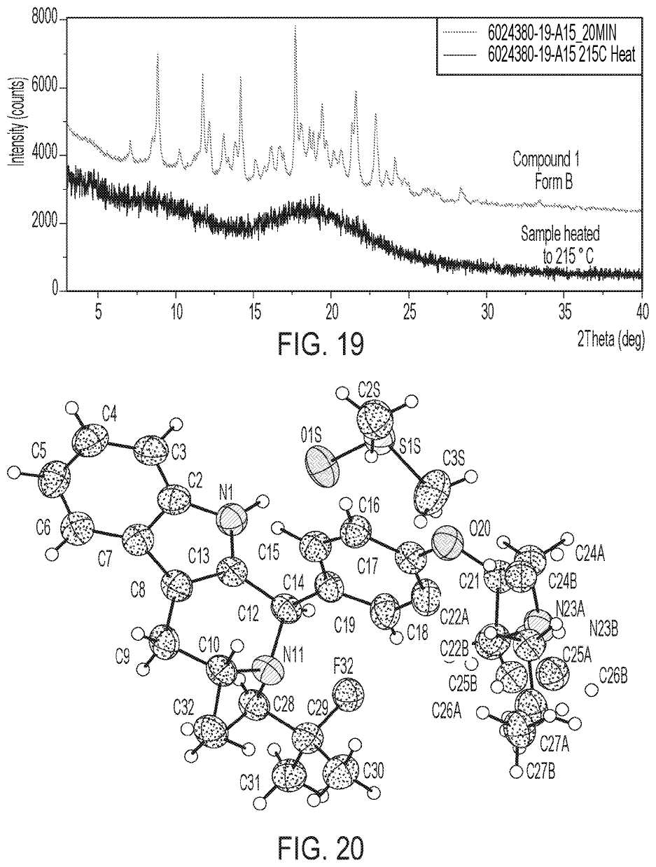

Upon heating Compound 1 Form B to 100° C., it became amorphous (FIG. 19).

Single crystals of Compound 1 Form B were grown serendipitously from DMSO. A Thermal Ellipsoid plot of the compound in the crystal is shown in FIG. 20. A rod-like single crystal with high diffraction quality, selected out from the batch, was immersed in MiTeGen LV5 (an oil based cryoprotectant) and mounted on a MiTeGen cryoloop in a random orientation and immersed in a stream of liquid nitrogen at 173K. The X-ray intensity data were measured on a Bruker D8 VENTURE (IμS microfocus X-ray source, Cu Kα, λ=1.54178 Å, PHOTON CMOS detector) diffractometer. The frames were integrated with the Bruker SAINT software package. The integration of the data using an orthorhombic unit cell in the space group P2₁2₁2₁ yielded a total of 22672 reflections to a maximum θ angle of 72.013° (0.83 Å resolution), of which 5703 were independent (Rᵢₙₜ=10.85%) and were greater than 2σ(F²). The final cell constants of α=10.2088(10)Å, b=10.8039(12)Å, c=26.539(3)Å, α=R=γ=90°, cell volume=2927.1(5) Å³, are based upon the refinement of the XYZ-centroids of 22724 reflections above 10 σ(I) with 3.331<θ<71.923°. Data were corrected for absorption effects using the Multi-Scan method (SADABS). The absorption coefficient μ of this material is 1.274 mm⁻¹ at this wavelength (1.54178 Å). The calculated minimum and maximum transmission coefficients (based on crystal size) are 0.862 and 974. The agreement factor for the structure (R₁) was 5.15% based on intensity. The single crystal parameters are summarized below:

| Empirical formula | C30 H42 F N3 O2 S | |
|---|---|---|
| Formula weight | 527.72 | |
| Temperature | 173(2) K | |
| Wavelength | 1.54178 Å | |
| Crystal system | Orthorhombic | |
| Space group | P2₁2₁2₁ | |
| Unit cell dimensions | a = 10.2088(10) Å | α = 90°. |
| | b = 10.8039(12) Å | β = 90°. |
| | c = 26.539(3) Å | γ = 90°. |

| | |
|---|---|
| Volume | 2927.1(5) Å³ |
| Z | 4 |
| Density (calculated) | 1.198 Mg/m³ |
| Absorption coefficient | 1.274 mm⁻¹ |
| F(000) | 1136 |
| Crystal size | 0.120 × 0.092 × 0.021 mm³ |
| Theta range for data collection | 3.330 to 72.013°. |
| Index ranges | −9 <= h <= 12, −13 <= k <= |
| | 13, −32 <= l <= 32 |
| Reflections collected | 22672 |
| Independent reflections | 5703 [R(int) = 0.1085] |
| Completeness to theta = 67.679° | 99.4% |
| Absorption correction | Semi-empirical from equivalents |
| Max. and min. transmission | 0.7539 and 0.5751 |
| Refinement method | Full-matrix least-squares on F² |
| Data/restraints/parameters | 5703/0/365 |
| Goodness-of-fit on F² | 1.032 |
| Final R indices [I > 2sigma(I)] | R1 = 0.0515, wR2 = 0.1318 |
| R indices (all data) | R1 = 0.0717, wR2 = 0.1514 |
| Absolute structure parameter | 0.025(18) |
| Extinction coefficient | 0.0036(6) |
| Largest diff. peak and hole | 0.232 and −0.270 e · Å⁻³ |

### Example 3: Preparation and Characterization of Compound 2 Solid Forms

### Compound 1 Malate Form A

Compound 1 Malate Form A was obtained according to the following exemplary procedure: (+)-D-Malic acid (120.8 mg) was weighed into a 20-mL glass vial. A 40 mg/mL solution of amorphous Compound 1 in ethyl acetate (15 mL) was added to the vial, and the mixture stirred at RT. A sample collected after 1 day of stirring was confirmed to be Compound 1 Malate Form A with XRPD. The resulting suspension was filtered, and the wet cake dried at 50° C. for 5 h under vacuum. Solids were collected to give Compound 1 Malate Form A (306.1 mg, ˜92.7% yield).

The XRPD pattern of Compound 1 Malate Form A is shown in FIG. 21, and the corresponding data are summarized below:

Position (degrees 2-Theta)

4.50
9.00
10.06
12.53
12.73
13.50
14.23
17.91
18.59
19.53
20.18
21.06
22.59
23.06
24.36
27.56

As shown by DSC curve in FIG. 22, the sample displayed one endothermic peak at 145.8° C. (peak temperature). FIG. 22 also shows the TGA curve, which shows a weight loss of 2.08% up to 150° C. Compound 1 Malate Form A was determined to be an anhydrate.

Analysis by ¹HNMR indicated a ˜2:1 stoichiometry of malic acid:Compound 1. (FIG. 23).

### Compound 1 Fumarate Form A Anhydrate

Compound 1 Fumarate Form A Anhydrate was obtained according to the following exemplary procedure: Fumaric acid (52.6 mg) was weighed into a 20-mL glass vial. A 40 mg/mL solution of amorphous Compound 1 in ethyl acetate (15 mL) was added to the vial, and the mixture stirred at RT. A sample collected after 1 day of stirring was confirmed to be Compound 1 Fumarate Form A Anhydrate with XRPD. The resulting suspension was filtered, and the wet cake dried at 50° C. for 5 h under vacuum. Solids were collected to give Compound 1 Fumarate Form A Anhydrate (231.9 mg, ˜92.2% yield).

The XRPD pattern of Compound 1 Fumarate Form A Anhydrate is shown in FIG. 24. Exemplary XRPD data for Compound 1 Fumarate Form A Anhydrate are summarized below:

| Position [°2Th.] | d-spacing [Å] | Rel. Int. [%] |
|---|---|---|
| 5.700345 | 15.50421 | 100.00 |
| 8.181217 | 10.80744 | 1.51 |
| 9.197129 | 9.61580 | 0.60 |
| 11.410820 | 7.75483 | 1.57 |
| 12.442390 | 7.11412 | 1.09 |
| 13.112900 | 6.75182 | 0.74 |
| 14.089020 | 6.28616 | 2.37 |
| 15.215930 | 5.82304 | 2.14 |
| 17.162930 | 5.16660 | 2.98 |
| 17.878770 | 4.96132 | 2.38 |
| 18.354670 | 4.83374 | 0.64 |
| 19.474810 | 4.55817 | 0.35 |
| 19.941570 | 4.45253 | 0.38 |
| 20.966210 | 4.23719 | 0.34 |
| 22.519120 | 3.94838 | 0.48 |
| 23.029790 | 3.86197 | 0.66 |
| 24.167500 | 3.68268 | 1.36 |
| 24.707800 | 3.60336 | 0.89 |
| 25.739760 | 3.46119 | 0.36 |
| 26.383470 | 3.37819 | 0.48 |
| 27.781990 | 3.21123 | 0.26 |
| 29.403730 | 3.03770 | 0.39 |
| 30.059080 | 2.97295 | 0.20 |

As shown by DSC curve in FIG. 25, the sample displayed one endothermic peak at 150.1° C. (onset temperature). FIG. 25 also shows the TGA curve, which shows a weight loss of 0.54% up to 150° C.

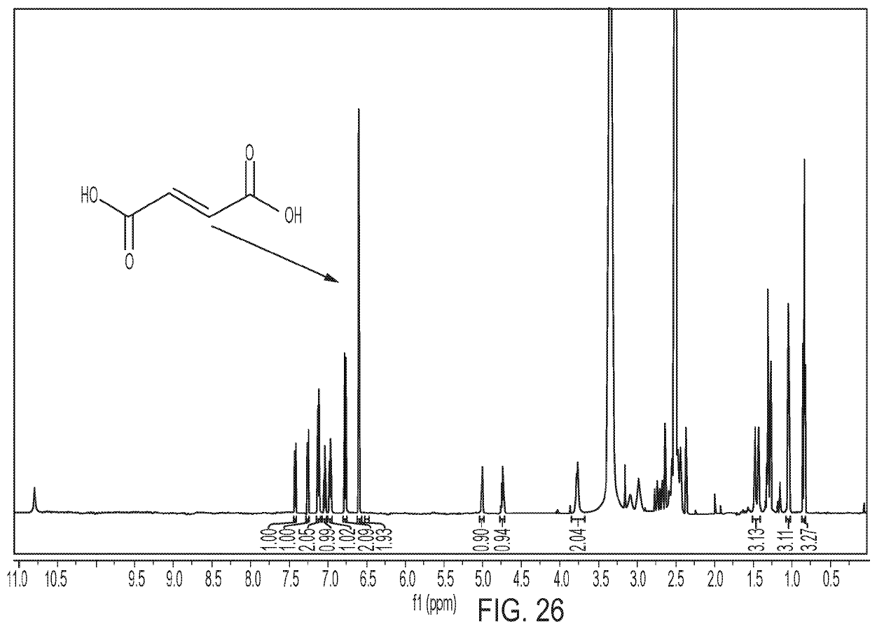

Analysis by ¹HNMR indicated a ˜1:1 stoichiometry of fumaric acid:Compound 1. (FIG. 26).

Compound 1 Fumarate Form A Anhydrate was also obtained according to the following procedure: Amorphous Compound 1 (1.0 g) and ethyl acetate (25 mL) were added to a 50 mL reactor vessel. Compound 1 dissolved under 25° C., and the agitation rate was kept at 300 rpm (two-blade paddle). Fumaric acid (1.2 equiv) was added in one portion. After stirring for 10 min, a large amount of solid precipitated. The mixture was stirred for 15 h. The suspension was filtered, and the wet cake dried at 40° C. for 16 h in a vacuum oven to give Compound 1 Fumarate Form A Anhydrate (1.035 g, 82% yield).

An XRPD pattern of Compound 1 Fumarate Form A Anhydrate is shown in FIG. 42, and the corresponding data are summarized below:

Position (degrees 2-Theta)

5.774
8.226
9.251
11.474
12.502
13.199
13.540
13.952
14.158
15.280
16.394
17.231
17.950
18.399
19.236
19.529
19.984
20.389
21.043
22.584
23.178
24.194
24.758
25.154
25.771
26.473
27.910
28.899
29.509
34.915
38.933

As shown by DSC curve in FIG. 43, the sample displayed one endothermic peak at 147° C. (peak temperature). FIG. 43 also shows the TGA curve, which shows a weight loss of 0.305% up to 160° C.

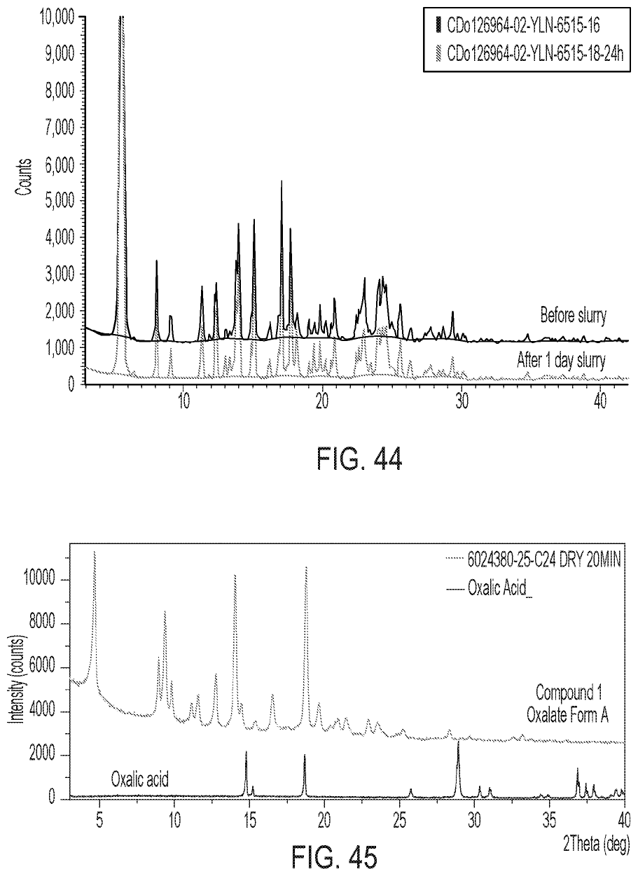

No change in form was observed after slurrying Compound 1 Fumarate Form A Anhydrate in isopropanol for 1 day (FIG. 44).

### Compound 1 Fumarate Form A Ethyl Acetate Solvate

Compound 1 Fumarate Form A Ethyl Acetate Solvate was prepared according to the following exemplary procedure: Amorphous Compound 1 (3.0025 g) was suspended in ethyl acetate (60 mL) resulting in a clear solution. Fumaric acid (774.6 mg) was added to the solution, an additional precipitation was observed. The mixture was stirred at ambient temperature for approximately a week. The solids formed were isolated by filtration via syringe with a positive displacement. Approximately 4.5 g of undried solids were recovered. The isolated material was consistent with Compound 1 Fumarate Form A Ethyl Acetate Solvate, as judged by XRPD (FIG. 27). Based on indexing of the XRPD pattern, the unit cell volume was consistent with a mono-ethyl acetate solvate of a mono-fumarate salt of Compound 1 (FIG. 28). The corresponding data are summarized below:

Position ±0.2 (degrees 2-Theta)

4.94
8.18
11.85
12.54
14.14
14.68
14.96
15.95
16.38
17.25
18.12
18.54
19.86
20.71

Solution ¹H NMR confirmed a 1:1 stoichiometry of Compound 1:fumaric acid. As shown by the DSC curve in FIG. 29, the sample displayed a broad endotherm at 89° C., an overlapping broad endotherm at 108° C., and a sharp endotherm with onset at 137° C./peak at 149° C. FIG. 29 also shows the TGA curve, which shows a weight loss of 14.4% between 28° C. and 130° C.

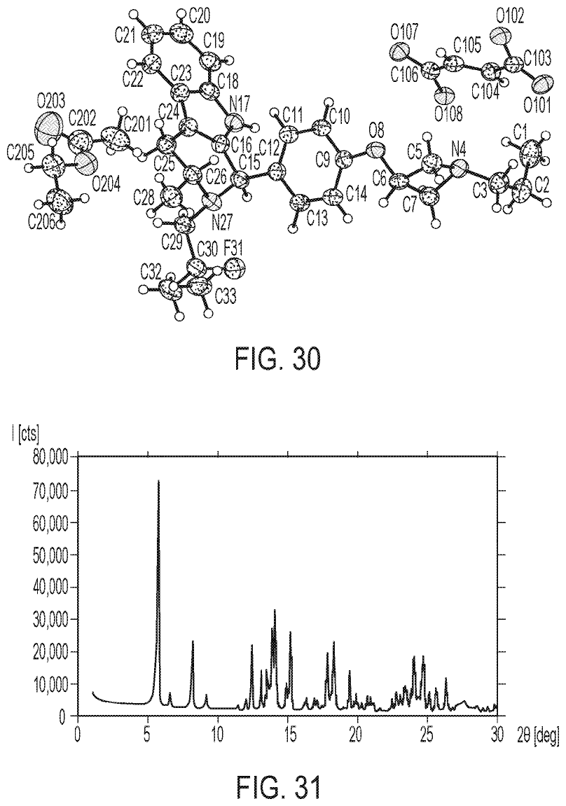

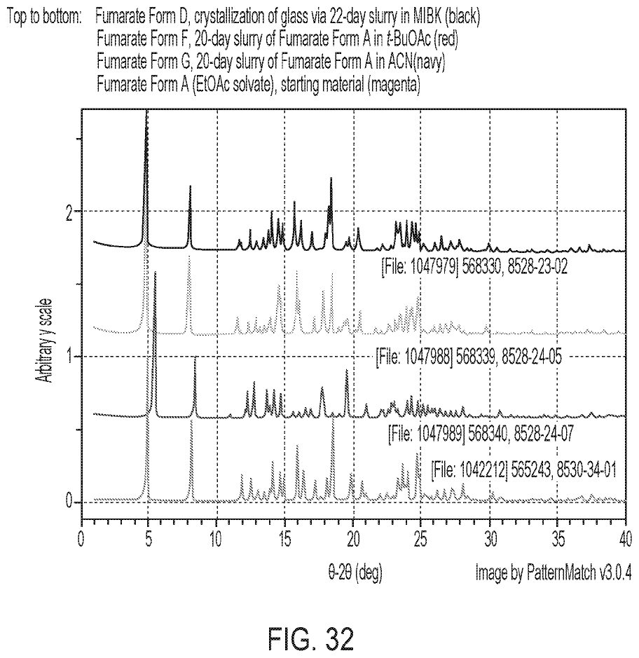

Single crystals of Compound 1 Fumarate Form A Ethyl Acetate Solvate were grown by slow cooling (60° C. to 5° C., in an incubator at ˜0.1° C./min) of a solution of Compound 1 Fumarate Form A Anhydrate (10.1 mg) in ethyl acetate (1 mL). A Thermal Ellipsoid plot of crystal structure is shown in FIG. 30. A block-like single crystal with high diffraction quality, selected out from the batch, was immersed in MiTeGen LV5 (an oil based cryoprotectant) and mounted on a MiTeGen cryoloop in a random orientation and immersed in a stream of liquid nitrogen at 173K. The X-ray intensity data were measured on a Bruker D8 VENTURE (I S microfocus X-ray source, Cu Kα, λ=1.54178 Å, PHOTON CMOS detector) diffractometer. The frames were integrated with the Bruker SAINT software package. The integration of the data using a monoclinic unit cell in the space group P2₁ yielded a total of 28237 reflections to a maximum θ angle of 74.2070 (0.83 Å resolution), of which 6809 were independent (Rᵢₙₜ=12.68%) and were greater than 2σ(F²). The final cell constants of α=7.444(5)Å, b=13.507(9)Å, c=17.766(16)Å, α=γ=90°, 3=92.73(4°), cell volume=1784(2) Å³, are based upon the refinement of the XYZ-centroids of 5257 reflections above 10 σ(I) with 2.491°<θ<72.989°. Data were corrected for absorption effects using the Multi-Scan method (SADABS). The absorption coefficient μ of this material is 0.719 mm⁻¹ at this wavelength (1.54178 Å). The calculated minimum and maximum transmission coefficients (based on crystal size) are 0.861 and 0.937. The agreement factor for the structure (R₁) was 7.42% based on intensity. The single crystal parameters are summarized below:

| Empirical formula | C36 H46 F N3 O7 | |
|---|---|---|
| Formula weight | 651.76 | |
| Temperature | 173(2) K | |
| Wavelength | 1.54178 Å | |
| Crystal system | Monoclinic | |
| Space group | P2₁ | |
| Unit cell dimensions | a = 7.444(5) Å | α = 90°. |
| | b = 13.507(9) Å | β = 92.73(4)°. |
| | c = 17.766(16) Å | γ = 90°. |

| | |
|---|---|
| Volume | 1784(2) Å³ |
| Z | 2 |
| Density (calculated) | 1.213 Mg/m³ |
| Absorption coefficient | 0.719 mm¹ |
| F(000) | 696 |
| Crystal size | 0.215 × 0.174 × 0.091 mm³ |
| Theta range for data collection | 2.490 to 74.207°. |
| Index ranges | −9 <= h <= 9, −15 <= k <= |
| | 16, −22 <= l <= 22 |
| Reflections collected | 28237 |
| Independent reflections | 6809 ┌R(int) = 0.1140┐ |
| Completeness to theta = 67.679° | 99.9% |
| Absorption correction | Semi-empirical from equivalents |
| Max. and min. transmission | 0.7538 and 0.5537 |
| Refinement method | Full-matrix least-squares on F² |
| Data/restraints/parameters | 6809/1/424 |
| Goodness-of-fit on F² | 1.031 |
| Final R indices [I > 2sigma(I)] | R1 = 0.0742, wR2 = 0.1943 |
| R indices (all data) | R1 = 0.0982, wR2 = 0.2211 |
| Absolute structure parameter | −0.01(16) |
| Extinction coefficient | n/a |
| Largest diff. peak and hole | 1.524 and −0.291 e · Å³ |

### Compound 1 Fumarate Form C

Compound 1 Fumarate Form C was obtained according to the following exemplary procedure: Compound 1 Fumarate Form A was placed in a vial, which was placed in a jar at 75% RH for 2 days. The resulting solids collected were Compound 1 Fumarate Form C.

The XRPD pattern of Compound 1 Fumarate Form C is shown in FIG. 31, and the corresponding data are summarized below:

Position (degrees 2-Theta)

5.67
6.52
8.14
9.14
11.38
11.95
12.40
13.07
13.44
13.84
14.04
15.16
17.82
19.45

Indexing of the XRPD pattern indicated that up to two molecules of water can be accommodated in the crystal lattice, suggesting that Compound 1 Fumarate Form C is likely hydrated.

### Compound 1 Fumarate Form D

Compound 1 Fumarate Form D was obtained according to the following exemplary procedure: Compound 1 Fumarate Form A (˜30-100 mg) was slurried in MIBK at ambient temperature for 20 days. Solids were isolated to give Compound 1 Fumarate Form D.

The XRPD pattern of Compound 1 Fumarate Form D is shown in FIG. 33, and the corresponding data are summarized below:

Position (degrees 2-Theta)

4.84
8.08
12.49
13.46
14.08
14.09
14.11
14.54
14.83
15.73
16.18
17.02
18.40
20.41

Unit cell volume obtained from indexing of the XRPD pattern suggested that Compound 1 Fumarate Form D was a MIBK solvate. Further, Compound 1 Fumarate Form D was determined to be isostructural with Compound 1 Fumarate Form A, Compound 1 Fumarate Form F, and Compound 1 Fumarate Form G, based on similarities in the XRPD patterns and unit cell parameters.

As shown by DSC curve in FIG. 34, the sample displayed one endothermic peak at 135° C. (onset)/146° C. (peak). FIG. 34 also shows the TGA curve, which shows a weight loss of 1.0% between 48° C. and 155° C.

### Compound 1 Fumarate Form E

Compound 1 Fumarate Form E was obtained according to the following exemplary procedure: Compound 1 Fumarate Form A (˜30-100 mg) was slurried in isopropanol at ambient temperature for 20 days. Solids were isolated to give Compound 1 Fumarate Form E.

The XRPD pattern of Compound 1 Fumarate Form E is shown in FIG. 35A.

As shown by DSC curve in FIG. 35B, the sample displayed one endothermic peak at 147° C. (onset)/156° C. (peak). FIG. 35B also shows the TGA curve, which shows no weight loss up to 180° C. Compound 1 Fumarate Form E was determined to be unsolvated.

Analysis by ¹HNMR indicated a ˜1:1 stoichiometry of fumaric acid:Compound 1.

Compound 1 Fumarate Form E was also prepared as follows: Amorphous Compound 1 (80.5 mg) and fumaric acid (24.4 mg) were mixed and suspended in isopropanol (2 mL) with stirring on a magnetic stirrer. Heptane (1 mL) was added to the clear solution, and the sample was placed in the freezer. After approximately one day, solids were isolated via centrifugation with filtration and analyzed by XRPD.

Compound 1 Fumarate Form E was also prepared as follows: Amorphous Compound 1 (2.0 g) and isopropanol (30 mL) were charged into a 50 mL reactor vessel. Compound 1 dissolved under 40° C., and the agitation rate was kept at 300 rpm (two-blade paddle). Fumaric acid (0.5 equiv) was added and dissolved after stirring for 5 min. Seeds of Compound 1 Fumarate Form E (1.0 wt %) were then added. After stirring for 1 h, fumaric acid (0.2 equiv) was added. After stirring for another 1 h, fumaric acid (0.2 equiv) was added. After stirring for another 1 h, fumaric acid (0.3 equiv) was added. Then, heptane (30 mL) was added within 4 h. The mixture was kept at 40° C. for 1 h, then cooled to 25° C. within 3 h, and then stirred for 10 h. The suspension was filtered, and the wet cake dried at 40° C. for 16 h in a vacuum oven to give Compound 1 Fumarate Form E (2.2 g, 90% yield).

Compound 1 Fumarate Form E was also prepared as follows: Amorphous Compound 1 (20.0 g) and isopropanol (300 mL) were charged into a 1000 mL reactor vessel. Compound 1 dissolved under 40° C., and the agitation rate was kept at 300 rpm (retreat curve impeller, RCI). Fumaric acid (0.5 equiv) was added and stirred for 20 min. Seeds of Compound 1 Fumarate Form E (1.0 wt %) were then added. After stirring for 1 h, fumaric acid (0.2 equiv) was added slowly. After stirring for another 1 h, fumaric acid (0.2 equiv) was added slowly. After stirring for another 1 h, fumaric acid (0.3 equiv) was added slowly. Then, heptane (300 mL) was added within 4 h. The mixture was kept at 40° C. for 1 h, then cooled to 25° C. within 3 h, and then stirred for 10 h. The suspension was filtered, and the wet cake dried at 40° C. for 16 h in a vacuum oven to give Compound 1 Fumarate Form E (23.2 g, 92% yield). Analysis by XRPD (FIG. 36A) confirmed that the material was Compound 1 Fumarate Form E. TGA analysis (FIG. 36B) showed a weight loss of 0.438% starting at 151.2° C., and DSC (FIG. 36C) showed a melting point of 157.9° C. (peak temperature). Gas chromatography (GC) indicated that isopropanol was present at 3264 ppm and heptane was present at 1434 ppm. DVS analysis (FIG. 36D) indicated that Compound 1 Fumarate Form E was not hygroscopic.

Exemplary XRPD data of Compound 1 Fumarate Form E are summarized below:

Position (degrees 2-Theta)

5.827
7.025
8.687
10.905
12.881
13.425
14.675
15.649
16.084
16.654
17.716
18.455
18.881
19.632
19.990
21.733
22.003
22.308
23.834
24.626
25.024
26.481
27.373
28.598
29.324
29.812
30.046
35.685
39.698
40.315
40.969

### Compound 1 Fumarate Form F

Compound 1 Fumarate Form F was obtained according to the following exemplary procedure: Compound 1 Fumarate Form A (˜30-100 mg) was slurried in t-BuOAc at ambient temperature for 20 days. Solids were isolated to give Compound 1 Fumarate Form F.

The XRPD pattern of Compound 1 Fumarate Form F is shown in FIG. 37, and the corresponding data are summarized below:

Position (degrees 2-Theta)

4.77
8.03
11.54
12.91
13.49
13.98
14.56
15.90
17.17
17.82
20.53
23.10
23.52
24.77

Unit cell volume obtained from indexing of the XRPD pattern suggested that Compound 1 Fumarate Form F was a tBuOAc solvate. Further, Compound 1 Fumarate Form F was determined to be isostructural with Compound 1 Fumarate Form A, Compound 1 Fumarate Form D, and Compound 1 Fumarate Form G, based on similarities in the XRPD patterns and unit cell parameters.

As shown by DSC curve in FIG. 38, the sample displayed a broad multi-peak endothermic event at 97° C., followed by an endotherm at 137° C. (onset)/145° C. (peak). FIG. 38 also shows the TGA curve, which shows a weight loss of 11.7% between 48° C. and 157° C.

### Compound 1 Fumarate Form G

Compound 1 Fumarate Form G was obtained according to the following exemplary procedure: Compound 1 Fumarate Form A (˜30-100 mg) was slurried in acetonitrile at ambient temperature for 20 days. Solids were isolated to give Compound 1 Fumarate Form G.

The XRPD pattern of Compound 1 Fumarate Form G is shown in FIG. 39, and the corresponding data are summarized below:

Position (degrees 2-Theta)

5.49
8.43
12.10
12.74
13.69
14.23
14.71
17.67
19.56
20.98

Unit cell volume obtained from indexing of the XRPD pattern suggested that Compound 1 Fumarate Form G was an acetonitrile solvate. Further, Compound 1 Fumarate Form G was determined to be isostructural with Compound 1 Fumarate Form A, Compound 1 Fumarate Form D, and Compound 1 Fumarate Form F, based on similarities in the XRPD patterns and unit cell parameters.

As shown by DSC curve in FIG. 40, the sample displayed an endotherm at 140° C. (onset)/149° C. (peak). FIG. 40 also shows the TGA curve, which shows a weight loss of 1.3% between 48° C. and 149° C.

### Compound 1 Fumarate Form J

Compound 1 Fumarate Form J was obtained according to the following exemplary procedure: The film obtained from slow evaporation of Compound 1 Fumarate Form A in MeOH/chloroform (17/83) was slurried in water at RT for 22 days. The solids obtained were Compound 1 Fumarate Form J.

The XRPD pattern of Compound 1 Fumarate Form J is shown in FIG. 41, and the corresponding data are summarized below:

Position (degrees 2-Theta)

4.37
5.34
8.28
9.18
9.53
9.77
11.79
13.81
17.03
18.66
19.19

### Compound 1 Oxalate Form A

Compound 1 Oxalate Form A was obtained according to the following exemplary procedure: Oxalic acid (82.2 mg) was weighed into a 20-mL glass vial. A 40 mg/mL solution of amorphous Compound 1 in ethyl acetate (15 mL) was added to the vial, and the mixture stirred at RT. A sample collected after 1 day of stirring was confirmed to be Compound 1 Oxalate Form A with XRPD. The resulting suspension was filtered, and the wet cake dried at 50° C. for 5 h under vacuum. Solids were collected to give Compound 1 Oxalate Form A (268.9 mg).

The XRPD pattern of Compound 1 Oxalate Form A is shown in FIG. 45, and the corresponding data are summarized below:

Position (degrees 2-Theta)

4.68
8.95
9.37
9.83
11.14
11.58
12.76
14.07
14.47
16.54
18.78
19.67
20.92
21.45
22.90
23.55
28.32

As shown by DSC curve in FIG. 46, the sample displayed two endothennic peaks at 104.2° C. and 196.8° C. (peak temperature). FIG. 46 also shows the TGA curve, which shows a weight loss of 3.78% up to 150° C. Compound 1 Oxalate Form A was determined to be an anhydrate.

### Compound 1 Phosphate Form A

Compound 1 Phosphate Form A was obtained according to the following exemplary procedure: A solution of amorphous Compound 1 and phosphoric acid (molar ratio of 1:1) in ethyl acetate was stirred at room temperature.

The XRPD pattern of Compound 1 Phosphate Form A is shown in FIG. 47, and the corresponding data are summarized below:

Position (degrees 2-Theta)

3.86
4.30
5.05
7.72
8.57
10.01
12.84
13.76
14.82
15.57
16.93
17.20
17.85
18.71
19.17
19.82
20.42
21.04
21.49
21.85
22.17
22.76
23.89
24.24
24.77
25.34
25.81
26.35
30.26
32.75

As shown by DSC curve in FIG. 48, the sample displayed one endotherm at 168.6° C. (onset temperature). FIG. 48 also shows the TGA curve, which shows a weight loss of 2.47% up to −100° C. Compound 1 Phosphate Form A was determined to be a hydrate.

### Compound 1 Phosphate Form B

Compound 1 Phosphate Form B was obtained according to the following exemplary procedure: A solution of amorphous Compound 1 and phosphoric acid (molar ratio of 1:1) in ethanol was stirred at room temperature.

The XRPD pattern of Compound 1 Phosphate Form B is shown in FIG. 49, and the corresponding data are summarized below:

Position (degrees 2-Theta)

3.29
4.38
6.54
7.72
8.70
9.04
10.93
13.07
14.89
16.94
17.47
19.70
21.18
23.76
24.10
25.08

As shown by DSC curve in FIG. 50, the sample displayed endotherms at 38.3° C., 132.9° C., and 156.1° C. (peak temperature). FIG. 50 also shows the TGA curve, which shows a weight loss of 3.98% up to −150° C. Compound 1 Phosphate Form B was determined to be either a solvate or a hydrate.

### Example 4: Polymorph Screening of Compound 1

Polymorph screening of Compound 1 was performed under 100 experimental conditions starting with amorphous Compound 1. A total of eight screening methods were used, including anti-solvent addition, reverse anti-solvent addition, slurry at 5° C., slurry at RT, slow evaporation, slow cooling, temperature cycling, and solid vapor diffusion. The results are summarized in Table 1, below:

TABLE 1

| Method | No. of Experiments | Crystal Form(s) Identified |
|---|---|---|
| Anti-solvent addition | 8 | Amorphous |
| Slow evaporation | 9 | Form A and Amorphous |
| Slow cooling | 11 | Form A and Amorphous |
| Slurry at RT | 30 | Form A, Form B, and Amorphous |
| Slurry at 5° C. | 11 | Form A and Amorphous |
| Solid vapor diffusion | 15 | Form A, Form B, and Amorphous |
| Reverse anti-solvent addition | 8 | Amorphous |
| Temperature cycling | 8 | Form A and Amorphous |
| Total | 100 | |

Anti-solvent addition experiments were conducted under 8 conditions, respectively. About 15 mg of Compound 1 was dissolved in 0.4-3.0 mL solvent to obtain a clear solution. The solution was magnetically stirred followed by addition of 0.1 mL anti-solvent per step for first 1 mL and adding 0.5 mL stepwise until precipitate appeared, or the total amount of anti-solvent reached 5.0 mL. The obtained precipitate was isolated for XRPD analysis. As summarized in Table 2, only amorphous Compound 1 was observed.

TABLE 2

| Solvent | Anti-Solvent | Form Observed |
|---|---|---|
| MeOH | H₂O | Amorphous |
| EtOH | H₂O | Amorphous |
| IPA | H₂O | Amorphous |
| Acetone | H₂O | Amorphous |
| THF | H₂O | Amorphous |
| ACN | H₂O | Amorphous |
| DMSO | H₂O | Amorphous |
| DMF | H₂O | Amorphous |

Slow evaporation experiments were conducted at RT under 9 different conditions. About 15 mg of Compound 1 was dissolved in 0.5 mL of solvent. All solutions and suspensions were filtered using a 0.45 m PTFE membrane the filtrates were used for the following steps. The visually clear solutions were covered by a HPLC cap with a hole in the cap created by a pipette tip and subjected to evaporation at room temperature. The solids were isolated for XRPD analysis. The result, summarized in Table 3, showed that Form A was obtained under certain conditions:

TABLE 3

| Solvent (v/v) | Form Observed |
|---|---|
| ACN | Form A |
| MeOH | Amorphous |
| EtOH | Amorphous |
| Acetone | Amorphous |
| THF | Gel |
| IPA | Gel |
| EtOAc | Gel |
| CHCl₃ | Gel |
| DCM | Gel |

Slow cooling experiments were conducted in 11 solvent systems, respectively. About 20 mg of Compound 1 was dissolved in 1.0-2.0 mL of solvent at 60° C. and filtered to a new vial using a 0.45 m PTFE membrane. Filtrates were slowly cooled down from 60° C. to 5° C. at a rate of 0.05° C./min. The obtained solids were kept isothermal at 5° C. before isolated for XRPD analysis. Anti-solvents were added into clear solutions to induce precipitation. Slow evaporation was conducted if no solid was observed after addition of anti-solvent. Results, summarized in Table 4, showed that Form A was obtained under certain conditions:

TABLE 4

| Solvent (v/v) | Form(s) Observed |
|---|---|
| ACN | Form A |
| MeOH/H₂O (1:4) | Amorphous* |
| EtOH/H₂O (1:4) | Amorphous* |
| Acetone/H₂O (1:4) | Form A + Amorphous* |
| ACN/H₂O (1:4) | Form A |
| THF/H₂O (1:4) | Form A + Amorphous* |
| MeOH/H₂O (1:9) | Amorphous* |
| EtOH/H₂O (1:9) | Amorphous* |
| Acetone/H₂O (1:9) | Amorphous* |
| ACN/H₂O (1:9) | Form A |
| THF/H₂O (1:9) | Amorphous* |

*Slow evaporation procedures used, as described above.

Slurry conversion experiments were conducted at RT in 30 solvent systems. About 20 mg of Compound 1 was suspended in 0.3 mL of solvent at RT for 4 days. The remaining solids were isolated for XRPD analysis. Results, summarized in Table 5, indicated that Form A and Form B were obtained under certain conditions:

TABLE 5

| Solvent (v/v) | Form Observed |
|---|---|
| ACN | Form A |
| DMSO | Form B |
| H₂O | Amorphous |
| DMSO/H₂O (1:1) | Amorphous |
| EtOH/H₂O (1:1) | Amorphous |
| Acetone/H₂O (1:1) | Form A |
| ACN/H₂O (1:1) | Form A |
| THF/H₂O (1:1) | Amorphous |
| DMSO/H₂O (1:4) | Amorphous |
| EtOH/H₂O (1:4) | Amorphous |
| Acetone/H₂O (1:4) | Amorphous |
| ACN/H₂O (1:4) | Form A |
| THF/H₂O (1:4) | Form A |
| DMSO/H₂O (1:9) | Amorphous |
| EtOH/H₂O (1:9) | Amorphous |
| Acetone/H₂O (1:9) | Amorphous |
| ACN/H₂O (1:9) | Form A |
| THF/H₂O (1:9) | Form A |
| ACN/H₂O (aw = 0.2, 989/11)* | Form A |
| ACN/H₂O (aw = 0.4, 978/22)* | Form A |
| ACN/H₂O (aw = 0.6, 959/41)* | Form A |
| ACN/H₂O (aw = 0.8, 925/75)* | Form A |
| Acetone/H₂O (aw = 0.2, 941/59)* | Form A |
| Acetone/H₂O (aw = 0.4, 857/143)* | Form A |
| Acetone/H₂O (aw = 0.6, 726/274)* | Form A |
| Acetone/H₂O (aw = 0.8, 492/508)* | Form A |
| DMSO/H₂O (aw = 0.2, 842/158) | Amorphous |
| DMSO/H₂O (aw = 0.4, 710/290) | Amorphous |
| DMSO/H₂O (aw = 0.6, 570/430) | Amorphous |
| DMSO/H₂O (aw = 0.8, 373/627) | Amorphous |

*Theoretical water activity based on software simulation.

Slurry conversion experiments were conducted at 5° C. in 11 solvent systems. About 30 mg of Compound 1 was suspended in 0.3 mL of solvent at 5° C. for 4 days. The remaining solids were isolated for XRPD analysis. Results, summarized in Table 6, indicated that Form A was obtained under certain conditions:

TABLE 6

| Solvent (v/v) | Form Observed |
|---|---|
| ACN | Form A |
| DMSO | Low Crystallinity* |
| ACN/H₂O (aw = 0.2, 989/11) | Form A |
| ACN/H₂O (aw = 0.4, 978/22) | Form A |
| ACN/H₂O (aw = 0.6, 959/41) | Form A |
| ACN/H₂O (aw = 0.8, 925/75) | Form A |
| DMSO/H₂O (1:4) | Amorphous |
| EtOH/H₂O (1:4) | Amorphous |
| Acetone/H₂O (1:4) | Form A |
| ACN/H₂O (1:4) | Form A |
| THF/H₂O (1:4) | Form A |

*Slow evaporation procedures used.

Solid vapor diffusion experiments were conducted using 12 different solvents, respectively. About 15 mg of Compound 1 was weighed into a 3-mL vial, which was placed into a 20-mL vial with 4 mL of volatile solvent. The 20-mL vial was sealed with a cap and kept at RT for 9 days allowing solvent vapor to interact with sample. The solids were tested by XRPD. The results, summarized in Table 7, showed that Form A and Form B were obtained under certain conditions:

TABLE 7

| Solvent | Form Observed |
|---|---|
| H₂O | Amorphous |
| MeOH | Amorphous |
| EtOH | Gel |
| IPA | Gel |
| Acetone | Gel |
| MEK | Gel |
| MIBK | Gel |
| IPAc | Gel |
| THF | Gel |
| 2-MeTHF | Gel |
| MTBE | Gel |
| DCM | Gel |
| ACN | Form A |
| Toluene | Gel |
| DMSO | Form B |

Reverse anti-solvent addition experiments were conducted in 8 solvent systems by first placing 1 mL of anti-solvcnt into a refrigerator at 5° C. in a 3 mL glass vial. About ˜10 mg of Compound 1 was then dissolved in 1 mL of solvent in a 2-mL glass vial. After the suspension was stirred magnetically for 2 hours yielding a clear solution, the solution was quickly filtered into the 5° C. antisolvent. The sample was then left at 5° C. to crystallize. If no crystallization occurred after 1 day, the sample was moved to −20° C. to precipitate. Remaining solids were isolated for XRPD analysis. The results, summarized in Table 8, showed that only amorphous API was obtained:

TABLE 8

| Solvent | Anti-Solvent | Form Observed |
|---|---|---|
| MeOH | H₂O | Amorphous |
| EtOH | H₂O | Amorphous |
| IPA | H₂O | Amorphous |
| Acetone | H₂O | Amorphous |
| THF | H₂O | Amorphous |
| ACN | H₂O | Amorphous |
| DMSO | H₂O | Amorphous |
| DMF | H₂O | Amorphous |

Temperature cycling experiments were conducted in 8 solvent systems. About 20 mg of Compound 1 was suspended in 0.1 mL of solvent in a 23-mL glass vial at RT. The suspension was then heated to 60° C., equilibrated for two hours. The slurry was slowly cooled down to 5° C. at a rate of 0.1° C./min and then heat to 60° C. in one hour. Repeat the cycle one more time and then cooling to 5° C. at a rate of 0.1° C./min. The samples were stored 5° C. before solids were isolated and analyzed using XRPD. Results summarized in Table 9 showed that Form A was obtained.

TABLE 9

| Solvent (v/v) | Form Observed |
|---|---|
| ACN | Form A |
| H₂O | Amorphous |
| EtOH/H₂O (1:4) | Amorphous |
| DMSO/H₂O (1:4) | Amorphous |
| ACN/H₂O (aw = 0.2, 989/11) | Form A |
| ACN/H₂O (aw = 0.4, 978/22) | Form A |
| ACN/H₂O (aw = 0.6, 959/41) | Form A |
| ACN/H₂O (aw = 0.8, 925/75) | Form A |

### Example 5: Salt Screening of Compound 1

Salt screening was conducted at room temperature (RT). A total of 100 salt screening experiments were conducted using 25 acids in 4 different solvent systems. Specifically, the stock solutions of Compound 1 are summarized in Table 10. The summary of the salt screen is presented in Table 11.

TABLE 10

| | | | | Volume used |
|---|---|---|---|---|
| | | Actual | Total | for each |
| | Temperature | sample size | volume | screening exp |
| Solvent (v/v) | (° C.) | (mg) | (mL) | (mL) |
| EtOH | RT | 610.1 | 15 | 0.5 |
| ACN | RT | 605.6 | 15 | 0.5 |
| EtOAc | RT | 611.7 | 15 | 0.5 |
| Acetone/H₂O | RT | 602.3 | 15 | 0.5 |
| (19:1, v:v) | | | | |

The stock solutions of Compound 1 were prepared in solvent systems at RT.
Sonication and heating were applied to the stock solutions until all Compound 1 was dissolved.

TABLE 11

| | Solvent | | | | |
|---|---|---|---|---|---|
| | | | | | Acetone/H₂O |
| Co-Former | pKa | EtOH | ACN | EtOAc | (19:1, v:v) |
| Blank | NA | Amorphous | Free Base Form A | Amorphous | Amorphous |
| (−)-L-Maleic acid (2:1) | 3.46 | Gel | Gel | Gel | Gel |
| (+)-D-Maleic acid (2:1) | 3.46 | Malate Form A | Gel | Malate Form A | Gel |
| (+)-L-Tartaric acid (2:1) | 3.02 | Gel | Gel | Gel | Gel |
| (−)-D-Tartaric acid (2:1) | 3.02 | Gel | Gel | Gel | Gel |
| Citric acid (2:1) | 3.13 | Gel | Gel | Citric Acid | Gel |
| D-Glucuronic Acid (2:1) | 3.18 | D-Glucuronic | D-Glucuronic | D-Glucuronic Acid | D-Glucuronic Acid |
| | | Acid | Acid | | |
| Fumaric acid (2:1) | 3.03 | Gel | Fumarate Form A | Fumarate Form A | Fumarate Form A |
| Glutamic acid (2:1) | 2.19 | Glutamic acid | Glutamic acid | Glutamic acid | Glutamic acid |
| Glycolic Acid (2:1) | 3.28 | Gel | Gel | Gel | Gel |
| H₃PO₄ (1:1) | 1.96 | Phosphate Form | Gel | Phosphate Form A | Gel |
| | | B | | | |
| H₃PO₄ (2:1) | 1.96 | Gel | Gel | Gel | Gel |
| HCl (1:1) | −6.00 | Gel | Gel | Gel | Gel |
| HCl (2:1) | −6.00 | Gel | Gel | Gel | Gel |
| Hippuric acid (2:1) | 3.55 | Gel | Hippuric acid | Hippuric acid | Gel |
| L-Aspartic acid (2:1) | 1.88 | L-Aspartic acid | L-Aspartic acid | L-Aspartic acid | L-Aspartic acid |
| Maleic acid (2:1) | 1.92 | Gel | Gel | Gel | Gel |
| Sulfuric acid (1:1) | −3.00 | Gel/Oil* | Oil* | Oil* | Gel/Oil* |
| Sulfuric acid (2:1) | −3.00 | Gel/Oil* | Oil* | Oil* | Gel/Oil* |
| (−)-L-Pyroglutamic acid | 3.32 | Gel | Gel | Gel | Gel |
| (2:1) | | | | | |
| Benzenesulfonic Acid | 0.70 | Gel | Gel | Gel | Gel |
| (2:1) | | | | | |
| Fumaric acid (1:1) | 3.03 | Gel | Fumarate Form A | Fumarate Form A | Gel |
| Malonic Acid (2:1) | 2.83 | Gel | Gel | Gel | Gel |
| Methanesulfonic acid | −1.20 | Gel | Gel | Gel | Gel |
| (2:1) | | | | | |
| Oxalic Acid (2:1) | 1.27 | Gel | Gel | Oxalate Form A | Oxalate Form A |
| Pamoic Acid (2:1) | 2.51 | Pamoic Acid | Pamoic Acid | Pamoic Acid | Pamoic Acid |

*Sample slurried for 1 hour at 5° C. and became black gel/oil.

All 14 hits were characterized by XRPD, TGA, DSC and solution NMR. The characterization results are summarized in Table 12.

TABLE 12

| | | | | Stoichiometry |
|---|---|---|---|---|
| | | | Purity | by NMR |
| | Weight Loss | DSC Endo. | by | (Counter Ion |
| Form | in TGA (%) | (Peak, ° C.) | HPLC | to Compound 1) |
| Malate | 1.58% | 60.7° C. and | ~100% | 2:1 |
| Form A | | 157.1° C. (peak) | | |
| Fumarate | 0.2% | 139.5° C. | ~100% | 1:1 |
| Form A | | | | |
| Anhydrate | | | | |
| Phosphate | 2.47% | 62.9° C. (peak), | 99.72% | ND |
| Form A | | 168.6° C. (onset) | | |
| | | and | | |
| | | 186.6° C. (peak). | | |
| Phosphate | 3.98% | 38.3° C., | 99.36% | ND |
| Form B | | 132.9° C. and | | |
| | | 156.1° C. (peak). | | |
| Oxalate | 0.97% | 94.0° C. (peak) | 99.77% | ND |
| Form A | | and | | |
| | | 125.7° C. (onset). | | |

ND = not determined.

Three salts were selected for further characterization—a malate salt, a fumarate salt, and an oxalate salt. The salts were scaled up to hundreds of milligrams. Characterization data are summarized in Table 13.

TABLE 13

| | | | Fumarate | |
|---|---|---|---|---|
| | Amorphous | Malate | Form A | |
| Crystal Form | Freebase | Form A | Anhydrate | Oxalate Form A |
| Crystallinity | Amorphous | High | High | Medium |
| Weight Loss in TGA (%) | 0.38% | 1.58% | 0.2% | 0.97% |
| Endotherm in DSC (° C., | 73.3 | 60.7, 157.1 | 139.5 | 94.0, 125.7 |
| peak) | | | | |
| HPLC purity (area %) | 100.00 | 100.00 | 100.00 | 99.77 |
| Stoichiometry | N/A | 2.06 | 0.965 | N/A |
| (acid: Compound 1) | | | | |
| Hygroscopicity (%)* | Slightly | Hygroscopic | Non-hygroscopic | Hygroscopic |
| | hygroscopic | (8.38) | (0.108) | (10.62) |
| | (0.463) | | | |
| Form Change after DVS? | No | No | No | No |

*Based on water uptake up at 25° C./80% RH: very hygroscopic: >15%, hygroscopic: 2-15%, slightly hygroscopic: 0.2-2%, non-hygroscopic: <0.2%.

### Example 6: Additional Polymorph Screening of Compound 1

A polymorph screen was conducted using amorphous Compound 1. To help design the experiments, kinetic solubilities of the compound were estimated. The estimation was done using a solvent aliquot addition method, and dissolution was judged by visual observation. Results are provided in Table 14. In Table 14, solvent ratios (v/v) are approximate; values are rounded to nearest whole number. If complete dissolution was achieved by one aliquot addition, solubilities were reported as “>”; if no solids were present, solubilities were reported as “<”. The actual solubility may be larger than the value calculated due to the use of solvent aliquots that were too large or due to a slow rate of dissolution.

TABLE 14

| Solvent System | Solubility (mg/mL) |
|---|---|
| ACN/water (60/40) | <6 |
| Dioxane/water (60/40) | <13 |
| Heptane | >191 |
| IPA | 82 |
| MeOH/water (40/60) | <55* |
| 2-MeTHF/MeOH/water (60/20/20) | 106 |
| NMP/water (68/32) | 7 |

*Appeared hydrophobic

Based on the solubility data, crystallization experiments were designed at micro (˜5-10 mg) and medium (˜30-80 mg) scales, utilizing techniques such as slow evaporation, slurry, and vapor stress of melts. Addition of crystalline seeds and a selected salt former were also explored. The experiments consisted of multiple steps, where observations from initial steps guided the approach to the subsequent steps.

Samples generated were visually observed by polarized light microscopy and analyzed by XRPD to perform a preliminary assessment. If solids produced exhibited a unique XRPD pattern, they were further characterized by solution H NMR to confirm the chemical composition and by TGA and DSC to evaluate the thermal behavior and the presence of volatiles. Conditions and results of the screen are summarized in Table 15.

TABLE 15

| Solvent System (Scale) | Conditions | Observations | XRPD Results |
|---|---|---|---|
| ACN | 1) Supernatant from | 1) Clear | Amount insufficient |
| | synthesis of Compound 1 | 2) Crystals | for XRPD |
| | Form A Acetonitrile Solvate | | |
| | 2) Slow evaporation | | |
| ACN/water (60/40) | 1) Added solvent | 1) Partially dissolved, turned | Amount insufficient |
| (microscale) | 2) Kept at RT, 9 days | viscous | for XRPD |
| | | 2) Crystalline, unknown | |
| | | morphology, BE | |
| BuOH dry | 1) Heated until liquefied, | 1) Crystalline, prismatic, BE | Amount insufficient |
| (medium scale) | cooled to RT, vapor stress | | for XRPD |
| | 1) Added solvent | 1) Dissolved | — |
| | 2) Slow evaporation | 2) Film | |
| DCE | 1) Added solvent | 1) Dissolved, no BE | — |
| (medium scale) | 2) Slow evaporation | 2) Film, no BE | |
| | 3) Scuffed bottom, kept at | 3) No change | |
| | RT | | |
| Dioxane/water (60/40) | 1) Added solvent | 1) Partially dissolved, turned | Amount insufficient |
| (microscale) | 2) Kept at RT, 9 days | viscous | for XRPD |
| | | 2) Crystalline, unknown | |
| | | morphology, BE | |
| Dioxane/water (60/40) | 1) Added solvent | 1) Viscous | Crystalline, similar to |
| (medium scale) | 2) Slurry, RT, 6 days | 2) Unknown morphology, BE | Compound 1 Form A |
| | | not apparent | Acetonitrile Solvate |
| | | | (Sample 15.1) |
| EtOAc dry | 1) Added solvent | 1) Dissolved, no BE | — |
| (medium scale) | 2) Slow evaporation | 2) Film, no BE | |
| | 3) Scuffed bottom, kept at | 3) Film | |
| | RT | | |
| | 1) Heated until liquefied, | 1) Potential crystalline, solution, | Crystalline, similar to |
| | cooled to RT, vapor stress | few BE speces | Compound 1 Form A |
| | 2) Slow evaporation | 2) Crystalline, BE, potential for | Acetonitrile Solvate |
| | 3) Attempted to sample | singles | (Sample 15.2) |
| | crystals for SCXRD | 3) Crystals broke | |
| EtOAc/heptane dry | 1) Added solvent | 1) Dissolved | — |
| (medium scale) | 2) Slow evaporation | 2) Film | |
| Heptane dry | 1) Heated until liquefied, | 1) — | — |
| (medium scale) | cooled to RT | 2) Dissolved, no BE | |
| | 2) Triturated with solvent | 3) Film, no BE | |
| | 3) Slow evaporation | 4) Film | |
| | 4) Scuffed bottom, kept at | | |
| | RT | | |
| | 1) Heated until liquefied, | 1) — | — |
| | cooled to RT | 2) Dissolved, no BE | |
| | 2) Vapor stress | 3) No solids | |
| | 3) Slow evaporation | | |
| | 1) Added solvent | 1) Dissolved, no BE | — |
| | 2) Fast evaporation, | 2) Film, no BE | |
| | elevated temperature | | |
| Isopropyl ether | 1) Added solvent | 1) Mostly clear, some viscous | — |
| | 2) Added crystal from Row | 2) Crystal remained | |
| | 1 (ACN experiment) | 3) No further change | |
| | 3) Kept at RT | | |
| IPA | 1) Added solvent | 1) Dissolved, no BE | — |
| (microscale) | 2) Kept at RT, 9 days | 2) Evaporated, film, no BE | |
| | 3) Scuffed bottom, kept at | 3) No change | |
| | RT | | |
| IPA | 1) Added solvent | 1) Dissolved, no BE | — |
| (medium scale) | 2) Slow evaporation | 2) Film | |
| | 1) Heated until liquefied, | 1) — | — |
| | cooled to RT | 2) Dissolved, no BE | |
| | 2) Vapor stress | 3) Film | |
| | 3) Slow evaporation | | |
| IPA/water (50/50) | 1) Added solvent | 1) Partially dissolved, turned | — |
| (medium scale) | 2) Kept at RT, 9 days | viscous | |
| | 3) Kept at RT | 2) Viscous, no BE | |
| | | 3) No change | |
| MeOH | 1) Added solvent | 1) Dissolved | — |
| (medium scale) | 2) Slow evaporation | 2) Viscous, no BE | |
| MeOH/water (40/60) | 1) Added solvent | 1) Appeared hydrophobic | — |
| (medium scale) | 2) Slurry, RT | 2) No BE | |
| 2-MeTH | 1) Heated until liquefied, | 1) — | — |
| (medium scale) | cooled to RT | 2) Dissolved, no BE | |
| | 2) Vapor stress | 3) Film, no BE | |
| | 3) Slow evaporation | | |
| | 1) Added solvent | 1) Dissolved, no BE | — |
| | 2) Slow evaporation | 2) Potential crystalline, film, few | |
| | 3) Scuffed bottom, kept at | BE particles | |
| | RT | 3) No change | |
| 2-MeTHF/MeOH/water | 1) Added solvent | 1) Dissolved, no BE | — |
| (60/20/20) | 2) Kept at RT, 9 days | 2) Partially evaporated, oil, no | |
| (microscale) | | BE | |
| MIBK dry | 1) Heated until liquefied, | 1) — | — |
| (medium scale) | cooled to RT | 2) Dissolved, no BE | |
| | 2) Vapor stress | 3) No solids | |
| | 3) Slow evaporation | | |
| MTBE | 1) Heated until liquefied, | 1) — | — |
| (medium scale) | cooled to RT | 2) Dissolved, no BE | |
| | 2) Vapor stress | 3) Film | |
| | 3) Slow evaporation | | |
| | 1) Added solvent | 1) Dissolved, no BE | — |
| | 2) Slow evaporation | 2) Potential crystalline, film, few | |
| | 3) Scuffed bottom, kept at | BE particles | |
| | RT | 3) No change | |
| NMP/water (68/32) | 1) Added solvent | 1) Dissolved, no BE | — |
| (microscale) | 2) Kept at RT, 9 days | 2) Partially evaporated, | |
| | 3) Sonicated, placed in | crystalline, unknown | |
| | freezer | morphology, BE | |
| | | 3) No change | |
| NMP/water (60/40) | 1) Added solvent | 1) Viscous | Crystalline, similar to |
| (medium scale) | 2) Slurry, RT, 6 days | 2) Crystalline, unknown | Compound 1 Form A |
| | | morphology, BE | Acetonitrile Solvate |
| | | | (Sample 15.3) |
| Water | 1) Heated until liquefied, | 1) — | — |
| (medium scale) | cooled to RT | 2) Glassy, no BE | |
| | 2) Vapor stress | 3) No change | |
| | 3) Kept at RT | | |
| Salt former addition | 1) Mixed with L-tartaric | 1) Remained | — |
| | acid | 2) Dissolved | |
| | 2) Added MeOH | 3) Evaporated, yellow viscous | |
| | 3) Slow evaporation | substance | |
| | 4) Added MTBE | 4) No change | |
| | 5) Added heptane | 5) Some solids | |
| | (MTBE/heptane, 29/71) | 6) Oil | |
| | 6) Slurry, RT, 11 days | 7) No change | |
| | 7) Decanted, added | | |
| | NMP/water, slurry | | |

BE = birefringence/extinction;
SCXRD = single crystal X-ray diffraction

Table 16 provides a summary of characterization data for the materials produced from this experiment. Sample numbers reference Table 15.

TABLE 16

| Sample | Analytical Technique | Results |
|---|---|---|
| Sample 15.2 | XRPD | Crystalline; single crystalline phase based on XRPD pattern |
| | | indexing; unit cell volume consistent with one molecule of |
| | | EtOAc per Compound 1 molecule. |
| | | Small peak at 5.4° 2θ due to Kapton. |
| Sample 15.1 | XRPD | Crystalline; single crystalline phase based on XRPD pattern |
| | | indexing; unit cell volume consistent with one molecule of |
| | | dioxane per Compound 1 molecule. |
| | | Small peak at 5.4° 2θ due to Kapton. |
| | ¹H NMR (collected 5 days | Consistent with chemical structure of Compound 1. |
| | after XRPD) | Dioxane (0.5 mol/mol) based on peak at 3.57 ppm. |
| | TGA (collected 5 days | Continuous loss of volatiles begins at 72° C.: 8.7 wt % loss, |
| | after XRPD) | 72-221° C., equivalent to 0.5 mol dioxane. |
| | | Weight loss, onset at 252° C. |
| | DSC (collected 5 days | Endotherm, peak max at 126° C. |
| | after XRPD) | Exotherm, above 200° C. |
| Sample 15.3 | XRPD | Crystalline; similar to Compound 1 Form A Acetonitrile |
| | | Solvate. |
| | | Small peak at 5.4° 2θ due to Kapton. |
| | ¹H NMR (collected 4 days | Consistent with chemical structure of Compound 1. |
| | after XRPD) | NMP (0.5 mol/mol) based on peaks at 2.69, 2.18, and 1.91 ppm |
| | TGA (collected 5 days | Continuous loss of volatiles begins at 89° C.: 9.0 wt % loss, |
| | after XRPD) | 89-239° C., equivalent to 0.5 mol NMP. |
| | | Weight loss, onset at 274° C. |
| | DSC (collected 5 days | Endotherm, peak max at 95° C. |
| | after XRPD) | Exotherm, above 200° C. |

### Example 7: Polymorph Screening of Compound 1 Fumarate

A polymorph screen was conducted using Compound 1 Fumarate Form A Ethyl Acetate Solvate. The screen consisted primarily of long term slurry experiments. To help design screen experiments, kinetic solubilities of Fumarate Form A Ethyl Acetate Solvate were estimated. The estimation was done on a 3-11 mg scale using a solvent aliquot addition method, and dissolution was judged by visual observation. Results are provided in Table 17. Solubilities are estimated at ambient temperature and reported to the nearest mg/mL; if complete dissolution was achieved by one aliquot addition, solubilites were reported as

TABLE 17

| Solvent System (Ratio v %) | Solubility (mg/mL) |
|---|---|
| ACN | <2* |
| Anisole | <1 |
| t-BuOAc | 1* |
| 2-BuOH | 17 |
| DCE | <9 |
| DCE/EtOH (96/4) | Dissolved at 8 |
| Dioxane | >104 |
| IPA | 20 |
| IPA/water (50/50) | >124 |
| 2-McTHF | 32 |
| MIBK | 4 |
| MTBE | 2 |
| TFE | >100 |

*Re-precipitated after first aliquot.

Long-term slurry experiments were conducted by stirring suspensions of Compound 1 Fumarate Form A Ethyl Acetate Solvate (˜30-100 mg) in various solvent systems at ambient temperature. Solvent systems were selected based on solubility estimations. After 20 days of stirring, solids were isolated by centrifugation with filtration (Table 18).

TABLE 18

| Solvent System | | | |
|---|---|---|---|
| (Ratio v %) | Conditions | Observations | XRPD Results |
| ACN | RT, 20 days | White solids | Fumarate Form G |
| tBuOAc | RT, 20 days | White solids | Fumarate Form F + |
| | | | Kapton peak at 5° 2θ |
| 2-BuOH | RT, 20 days | Light yellow | Fumarate Form E + |
| | | solids | Kapton peak at 5° 2θ |
| EtOH/DCE | RT, 20 days | White solids | Fumarate Form H + |
| (4/96) | | | Fumarate Form E |
| IPA | RT, 20 days | White solids | Fumarate Form E + |
| | | | Kapton peak at 5° 2θ |
| MEK/heptane | RT, 20 days | White solids | Fumarate Form H + |
| (40/60) | | | Kapton peak at 5° 2θ |
| 2-MeTHF | RT, 20 days | White solids | Fumarate Form H |
| MIBK | RT, 20 days | Light yellow | Fumarate Form D |
| | | solids | |
| MTBE | RT, 20 days | White solids | Fumarate Form H |
| NMP/water | RT, 20 days | White solids | Fumarate Form E + |
| (40/60) | | | Kapton peak at 5° 2θ |

Kinetic experiments using ˜30-50 mg Compound 1 Fumarate Form A Ethyl Acetate Solvate included crystallization techniques such as fast and slow evaporation; solvent/antisolvent precipitation with ripening; crystallization at subambient temperature; and organic and aqueous vapor stress (Table 19). For evaporation experiments, filtered solutions of test material were left uncapped at ambient temperature for fast evaporation or covered with aluminum foil with pin holes for slow evaporation. For solvent/antisolvent precipitation, solutions of starting material were prepared at ambient or elevated temperature and filtered using a 0.2 μm nylon filter. The solutions were mixed with appropriate antisolvents via a direct or reverse addition. Solids precipitated were either immediately isolated by vacuum filtration or left at ambient temperature for ripening. For crystallization at subambient temperature, solutions of starting material were prepared at ambient temperature and filtered using a 0.2 μm nylon filter. The filtered solutions were then placed at subambient conditions for slow crystallization. Solids precipitated were isolated via centrifugation with filtration. For vapor stress experiments, solids of starting material were sampled in vials, which were placed in a RH jar (prepared as described in Greenspan, L., Journal of Research of the National Bureau of Standards Section A: Physics and Chemistry, vol. 81 Å, no. 1, 1977, p. 89, doi:10.6028/jres.081a.011) at set temperature or a secondary container with water. After a specified duration, samples were collected and analyzed.

TABLE 19

| Solvent System | | | |
|---|---|---|---|
| (Ratio v %) | Conditions | Observations | XRPD Results |
| 2-BuOH | Crystallization in freezer in | Clear | — |
| | attempt to grow suitable | | |
| | crystals (6 mg scale) | | |
| Dioxane/hexanes | Solvent/antisolvent | Unknown | Fumarate Form H |
| (8/92) | precipitation with ripening | morphology, sticky, | |
| | | some BE | |
| DCE/EtOH (96/4) | Slow evaporation in attempt to | Crystals with BE; not | — |
| | grow suitable crystals (11 mg | suitable for SCXRD | |
| | scale) | | |
| EtOH/nitromethane | Slow evaporation | Film, no BE | — |
| (3/97) | | | |
| EtOH/toluene (9/91) | Slow evaporation | Unknown | Fumarate Form B + Kapton |
| | | morphology, BE | peaks at 5, 15, and 29° 2θ |
| IPA/heptane (17/83) | Supernatant from Sample 22.2; | Clear | — |
| | crystallization in freezer | | |
| IPA/heptane (67/33) | Supernatant from Sample 22.1; | Clear | — |
| | crystallization in freezer | | |
| MeOH | Slow evaporation | Glass | — |
| | (concentration 19 mg/mL) | | (Sample 19.1) |
| | Slow evaporation | Glass | — |
| | (concentration 26 mg/mL) | | (Sample 19.2) |
| MeOH/chloroform | Slow evaporation | Film, no BE | — |
| (17/83) | | | (Sample 19.3) |
| 2-MeTHF | Slow evaporation | Film, no BE | — |
| | | | (Sample 19.4) |
| | Slow evaporation (3 mg scale) | BE | Fumarate Form C, selected |
| | | | peaks slightly shifted + Kapton |
| | | | peak at 5, 15, and 29° 2θ |
| MIBK | Crystallization in freezer (4 mg | Clear | — |
| | scale) | | |
| MTBE | Supernatant from Sample 22.3; | Clear | — |
| | crystallization in freezer | | |
| TFE | Fast evaporation | Film, no BE | — |
| | | | (Sample 19.5) |
| — | Water vapor stress, 1 day | — | Fumarate Form C, selected |
| | | | peaks, slightly shifted |
| — | 75% RH stress, 2 days | — | Fumarate Form C |

BE = birefringence/extinction;
SCXRD = single crystal X-ray diffraction

Crystallization of glasses and films obtained from the kinetic experiments was conducted via stirring for approximately 22 days (Table 20). Sample numbers reference Table 19.

TABLE 20

| Starting | Conditions | | |
|---|---|---|---|
| Material | (Solvent Ratio v %) | Observations | XRPD Results |
| Sample 19.1 | MTBE slurry, RT, 22 days | Dry, yellow solids | Fumarate Form H + |
| | | | Kapton peak at 5° 2θ |
| Sample 19.2 | MIBK slurry, RT, 22 days | Yellow solids | Fumarate Form D + |
| | | | Kapton peak at 5° 2θ |
| Sample 19.3 | Water slurry, RT, 22 days | Yellow solids | Fumarate Form J, |
| | | | disordered + Kapton |
| | | | peak at 5° 2θ |
| Sample 19.5 | IPA/heptane (22/78) slurry, | Yellow solids | Fumarate Form E + |
| | RT, 22 days | | Kapton peak at 5° 2θ |
| Sample 19.4 | 2-BuOH slurry, RT, 22 days. | No solids | — |
| | Concentrated by slow | | |
| | evaporation. | | |

Table 21 provides a summary of the characterization data for the materials produced from this experiment.

TABLE 21

| Material Designation | Analytical Technique | Results |
|---|---|---|
| Fumarate Form E | XRPD | Crystalline, single crystalline phase, unit cell volume |
| (produced in IPA) | | from XRPD indexing is consistent with unsolvated |
| | | mono-fumarate salt. Broad feature near 5° 2θ due to |
| | | Kapton film. |
| | PLM | Solids were placed on a microscope slide, covered by |
| | | a coverslip. Mineral oil was added through capillary |
| | | action. |
| | | Loose and agglomerated particles with BE. Individual |
| | | particles appear primarily as blades with some |
| | | particles being flaky and anhedral. Various sizes of |
| | | agglomerates, which appear to be hard upon light |
| | | pressure. |
| | ¹H NMR (6 days after XRPD) | Consistent with Compound 1 fumarate with 1:1 |
| | | stoichiometry. Organic solvents are not apparent. |
| | TGA (10 days after XRPD) | Weight loss, onset at 80° C. |
| | DSC (10 days after XRPD) | Strong endotherm, onset at 147° C. and peak max at |
| | | 156° C. |
| Fumarate Form E | XRPD | Crystalline, minor shifts of selected XRPD peaks |
| (produced in 2-BuOH) | | compared to Fumarate Form E generated in IPA. |
| | | Broad feature near 5° 2θ due to Kapton film. |
| | ¹H NMR (1 month after XRPD) | Consistent with Compound 1 fumarate with 1:1 |
| | | stoichiometry. Trace 2-BuOH possible. |
| | TGA (3 weeks after XRPD) | 6.3 wt % loss, 29-92° C. (equivalent to 2 mol water). |
| | | Weight loss, onset at 179° C. |
| | DSC (3 weeks after XRPD) | Weak broad and asymmetric endotherm, peak max at |
| | | 70° C. Strong endotherm, onset at 147° C. and peak |
| | | max at 155° C. |
| Fumarate Form E | XRPD | Crystalline, XRPD peak shifts are not apparent |
| (produced in | | compared to Fumarate Form E generated in IPA. |
| NMP/water 40/60) | | Broad feature near 5° 2θ due to Kapton film. |
| | ¹H NMR (1 month after XRPD) | Consistent with Compound 1 fumarate with 1:1 |
| | | stoichiometry. NMP (1 mol/mol), based on peaks at |
| | | 3.3, 2.2, and 1.9 ppm. |
| | TGA (3 weeks after XRPD) | 1.5 wt % loss, 35-91° C. (equivalent to 0.5 mol water) |
| | | Weight loss, onset at 178° C. |
| | DSC (3 weeks after XRPD) | Broad endotherm, peak max at 80° C. |
| Fumarate Form D | XRPD | Crystalline, single crystalline phase, unit cell volume |
| | | from XRPD indexing is consistent with mono-MIBK |
| | | solvate of mono-fumarate salt of Compound 1. Broad |
| | | feature near 5° 2θ due to Kapton film. |
| Fumarate Form D | XRPD | Crystalline, single crystalline phase |
| | ¹H NMR (6 days after XRPD) | Consistent with Compound 1 fumarate with 1:1 |
| | | stoichiometry. MIBK (0.7 mol/mol), based on peaks at |
| | | 2.3, 2.1, 2.0, and 0.9 ppm. |
| | TGA (10 days after XRPD) | 1.0 wt % loss, 48-155° C. (equivalent to 0.06 mol of |
| | | MIBK or 0.5 mol of water). |
| | | Weight loss, onset at 180° C. |
| | DSC (10 days after XRPD) | Strong endotherm, onset at 135° C. and peak max at |
| | | 146° C. |
| Fumarate Form F | XRPD | Crystalline, single crystalline phase, unit cell volume |
| | | from XRPD indexing is consistent with mono- |
| | | tBuOAc solvate of mono-fumarate salt of Compound |
| | | 1. Broad feature near 5° 2θ due to Kapton film. |
| | ¹H NMR (6 days after XRPD) | Consistent with Compound 1 fumarate with 1:1 |
| | | stoichiometry. tBuOAc (0.7 mol/mol), based on peaks |
| | | at 1.9 and 0.39 ppm. |
| | TGA (10 days after XRPD) | 11.7 wt % loss, 48-157° C. (equivalent to 0.6 mol |
| | | tBuOAc) |
| | | Weight loss, onset at 180° C. |
| | DSC (10 days after XRPD) | Small broad endotherms, peak max at 97° C. |
| | | Strong endotherm, onset at 137° C. and peak max at |
| | | 145° C. |
| Fumarate Form G | XRPD | Crystalline, single crystalline phase, unit cell volume |
| | | from XRPD indexing is consistent with mono-ACN |
| | | solvate of mono-fumarate salt of Compound 1. |
| | ¹H NMR (6 days after XRPD) | Consistent with Compound 1 fumarate with 1:1 |
| | | stoichiometry. ACN (0.3 mol/mol), based on peak at |
| | | 2.07 ppm. |
| | TGA (10 days after XRPD) | 1.3 wt % loss, 48-149° C. (equivalent to 0.2 mol ACN). |
| | | Weight loss, onset at 180° C. |
| | DSC (10 days after XRPD) | Strong endotherm, onset at 140° C. and peak max at |
| | | 149° C. |
| Fumarate Form C | XRPD | Crystalline, single crystalline phase, unit cell volume |
| | | from XRPD indexing is consistent with mono- |
| | | fumarate salt of Compound 1 containing up to 2 mol |
| | | of water. |

Salt formation experiments were also performed by stirring mixtures of Amorphous Compound 1 and fumaric acid (1:1.2 ratio). Solids precipitated were isolated via centrifugation with filtration. Results are summarized in Table 22.

TABLE 22

| Conditions (Solvent Ratio v %) | XRPD Results |
|---|---|
| 1) Suspended 81 mg of Compound 1 and 24 mg of fumaric acid in 2 | Fumarate Form E, |
| mL of IPA with stirring (clear) | slightly disordered + |
| 2) Added 1 mL of heptane (clear) | Kapton peak at 5° 2θ |
| 3) Placed in freezer, 1 day | (Sample 22.1) |
| 4) Isolated cold via centrifugation with filtration (greasy pasty solids) | |
| 5) Air dried (loose solids) | |
| 1) Suspended 81 mg of Compound 1 and 26 mg of fumaric acid in 1 | Fumarate Form C, |
| mL IPA with stirring and placed on hot plate (clear) | shifted |
| 2) Added 5 mL of heptane by aliquots (some precipitation) | (Sample 22.2) |
| 3) Cooling to RT (partially oiled out) | |
| 4) Sonicated, 1 hour (solids, no oil) | |
| 5) Stirring, RT, 1 day | |
| 6) Isolated via centrifugation with filtration (greasy, pasty solids) | |
| 7) Air dried (loose solids) | |
| 1) Suspended 80 mg Compound 1 and 26 mg fumaric acid in 2 mL of | Fumarate Form H + |
| MTBE with stirring (some dissolution, then additional precipitation) | fumaric acid |
| 2) Stirring, RT, 1 day | (Sample 22.3) |
| 3) Isolated via centrifugation with filtration (loose solids) | |

### Example 8: Stability Studies

Further evaluation of hygroscopicity and water solubility was conducted to better understand the physicochemical properties of Compound 1 Malate Form A, Compound 1 Fumarate Form A Anhydrate, and Compound 1 Oxalate Form A.

### Hygroscopicity

A DVS isotherm plot was collected at 25° C. to investigate the solid form stability as a function of humidity. For Compound 1 Fumarate Form A Anhydrate, solids were pre-equilibrated at 0% RH to remove the unbounded solvent or water before the hygroscopicity experiment was started. For Compound 1 Malate Form A and Compound 1 Oxalate Form A, solids were equilibrated at ambient humidity (˜40% RH).

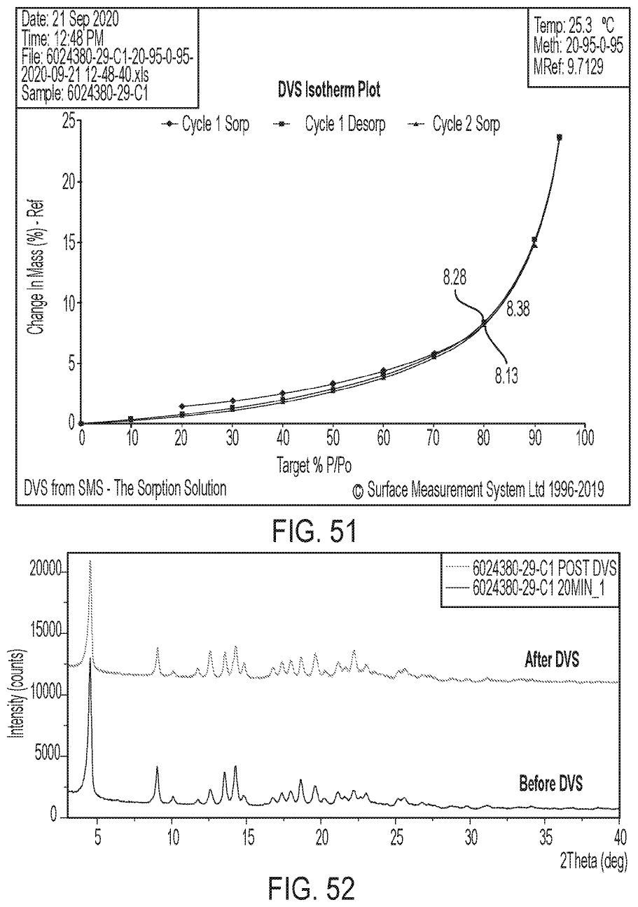

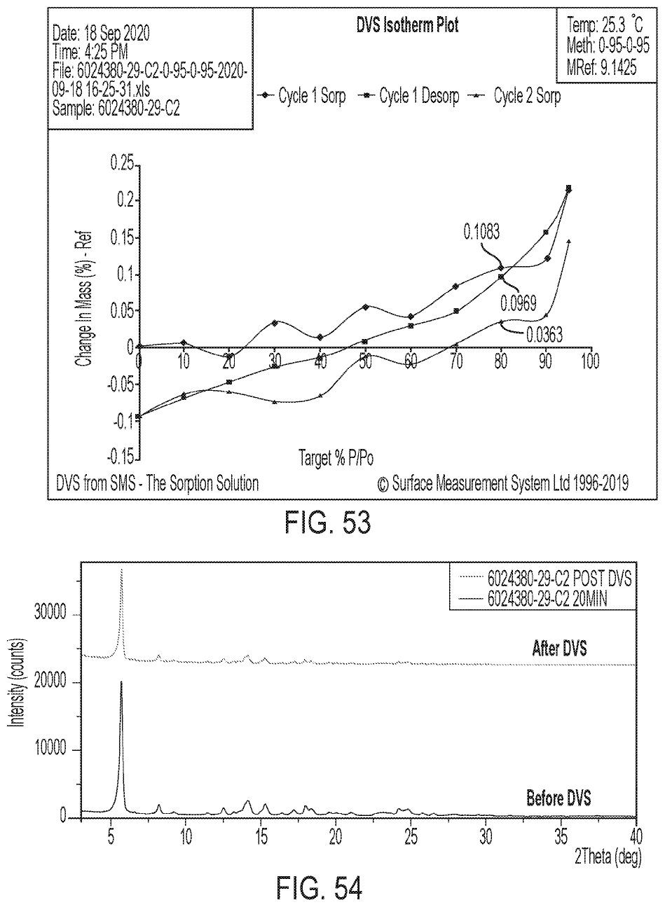

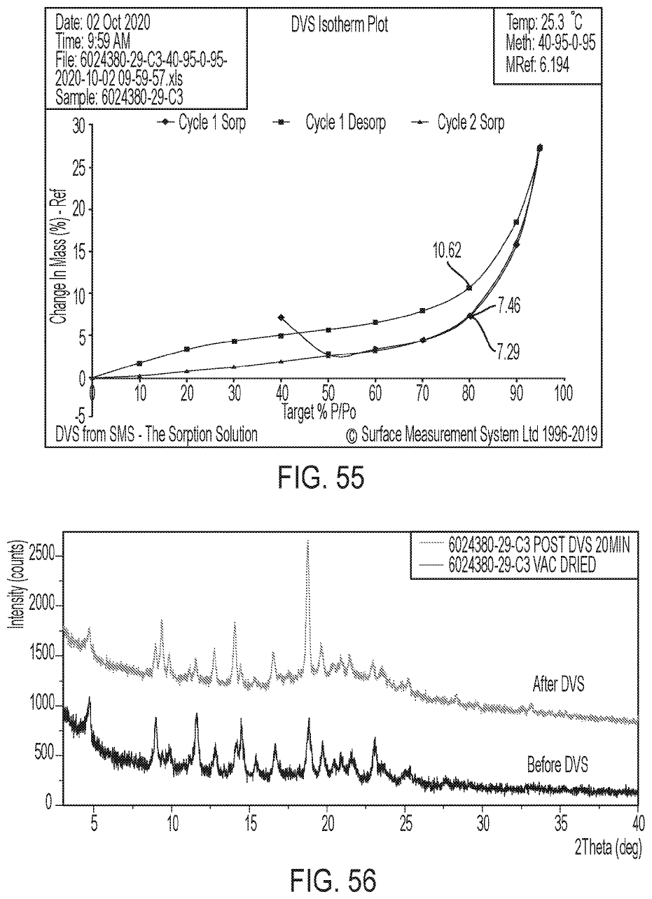

As evidenced by the water uptake of 2.00-15.00% up to 80% RH, Compound 1 Malate Form A and Compound 1 Oxalate Form A were hygroscopic. Compound 1 Fumarate Form A Anhydrate was found to be non-hygroscopic, showing a water uptake of less than 0.20% up to 80% RH. No solid form change was observed for any the three test forms after DVS evaluation, see FIGS. 51 to 56.

### Water Solubility

Water solubility was measured in water at RT to evaluate solubility and disproportionation risk. All solubility samples (initial solid loading of ˜10-40 mg/mL) were kept stirring at 400 rpm and sampled after 24 hours. After centrifugation, supernatants were collected for HPLC and pH tests, and wet cakes were collected for XRPD characterization. The results were summarized in Table 23. No form change was observed in any of the tested forms.

TABLE 23

| | | | Form Change | |
|---|---|---|---|---|
| Form | Solubility in Water | pH | after 24 h? | Purity |
| Malate Form A | 10.69 mg/mL | 4.19 | No | 93.93% |
| Fumarate Form A | 2.23 mg/mL | 5.24 | No | ~100% |
| Anhydrate | | | | |
| Oxalate Form A | 26.93 mg/mL | 2.84 | No | 89.60% |

### Example 9: Solubility Studies

Solubility studies of Compound 1 and Compound 1 Fumarate in different solvents at 25° C. were performed. Solubility was measured by the dynamic method and gravimetric method.

Dynamic method: Under the condition of a certain amount of solute and certain temperature, solvent was gradually added with stirring for 15 min to reach equilibrium. When solute is completely dissolved, amount of solvent was recorded and the solubility calculated.

Gravimetric method: Excess solids and a certain amount of solvent were added 8 mL vials, stirred for 24 hours, and filtered. 1 mL of clear upper layer of liquid was taken, dried at 50° C. for 24 h, weighed, and the solubility calculated.

The results are summarized in Table 24. Amorphous Compound 1 had high solubility in isopropanol, and fumaric acid had relatively low solubility in isopropanol. Compound 1 Fumarate Form E had very low solubility in isopropanol at 25° C., and the solubility decreased significantly with increasing proportion of heptane. Compound 1 Fumarate Form E had much lower solubility than Compound 1 Fumarate Form A Anhydrate, indicating that Form E is more stable than Form A.

TABLE 24

| Material | Method | Solvent | Solubility (mg/mL) | |
|---|---|---|---|---|
| Amorphous | Dynamic | IPA | >467.5 | |
| Compound 1 | | | | |
| Fumaric acid | Dynamic | IPA | 34.3 | |
| Compound 1 | Dynamic | IPA | ~8.46 | (25° C.) |
| Fumarate Form E | Dynamic | IPA | <1 | (0° C.) |

| | | | |
|---|---|---|---|
| | Dynamic | IPA/heptane (5/1) | 5.89 |
| | Dynamic | IPA/heptane (5/3) | ~3.57 |
| | Dynamic | IPA/heptane (1/1) | ~2.35 |
| | Dynamic | IPA/heptane (1/3) | <1 |
| | Gravimetric | H₂O | 1.0 |
| Compound 1 | Gravimetric | H₂O | 6.5 |
| Fumarate Form A | | | |
| Anhydrate | | | |

### Example 10: Bulk Density Studies

Bulk density of Compound 1 Fumarate Form E was measured by gently introducing a known sample mass into a graduated cylinder (50 mL), leveling the powder without compacting it, and recording the apparent untapped volume to the nearest graduated unit. The experiment was repeated three times, and the results are summarized in Table 25.

TABLE 25

| Run | Bulk Density (g/mL) |
|---|---|
| 1 | 0.1288 |
| 2 | 0.1233 |
| 3 | 0.1196 |
| Average | 0.1239 |

### Example 11: Additional Stability Studies

Amorphous Compound 1, Compound 1 Fumarate Form E, and Compound 1 Fumarate Form A Anhydrate were placed in an oven at 60° C. for two weeks to evaluate their stability. As shown in Table 26, Compound 1 Fumarate Form E and Compound 1 Fumarate Form A Anhydrate exhibited improved stability relative to Amorphous Compound 1.

TABLE 26

| | HPLC Purity (%) | | | |
|---|---|---|---|---|
| Material | Starting | 60° C. | Degradation | XRPD |
| Amorphous Compound 1 | 99.23% | 98.94% | −0.29% | No change |
| Compound 1 Fumarate | 99.54% | 99.50% | −0.05% | No change |
| Form E | | | | |
| Compound 1 Fumarate | 99.58% | 99.54% | −0.04% | No change |
| Form A Anhydrate | | | | |

Competitive slurry experiments were performed with Compound 1 Fumarate Form E and Compound 1 Fumarate Form A Anhydrate, as follows:

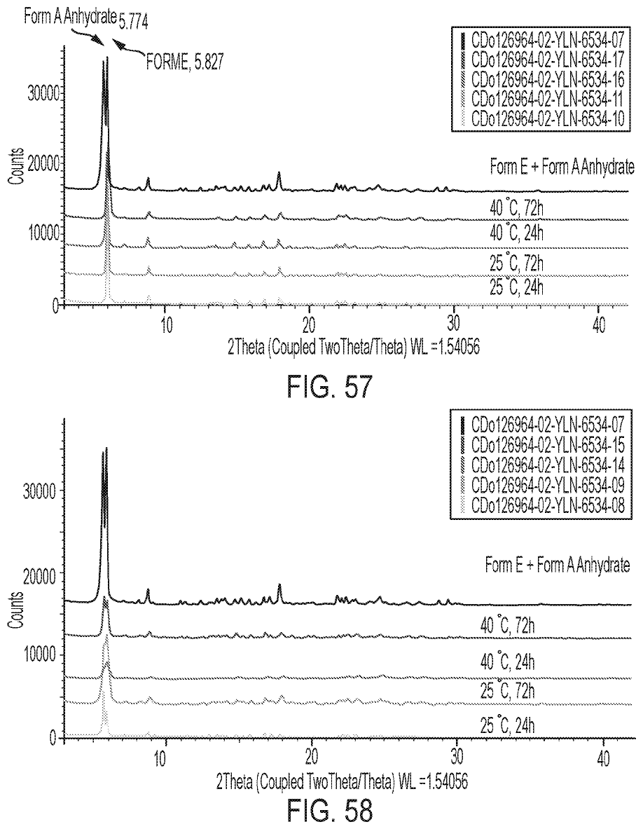

Experiment #1: Compound 1 Fumarate Form E (100 mg), Compound 1 Fumarate Form A Anhydrate (100 mg), and isopropanol (2 mL) were added into two 8-mL vials and mixed well in a shaker at 25° C. and 40° C., respectively. Samples for analysis were taken at 24 h and 72 h. Results of XRPD analysis are shown in FIG. 57. After 24 h at either 25° C. or 40° C., Compound 1 Fumarate Form A Anhydrate completely transformed into Compound 1 Fumarate Form E, indicating that Form E is more stable than Form A in isopropanol.

Experiment #2: Compound 1 Fumarate Form E (100 mg), Compound 1 Fumarate Form A Anhydrate (100 mg), and water (2 mL) were added into two 8-mL vials and mixed well in a shaker at 25° C. and 40° C., respectively. Samples for analysis were taken at 24 h and 72 h. Results of XRPD analysis are shown in FIG. 58. After 72 h at either 25° C. or 40° C., a mixture of Compound 1 Fumarate Form E and Compound 1 Fumarate Form A Anhydrate remained, indicating that conversion between the forms is very slow in water.

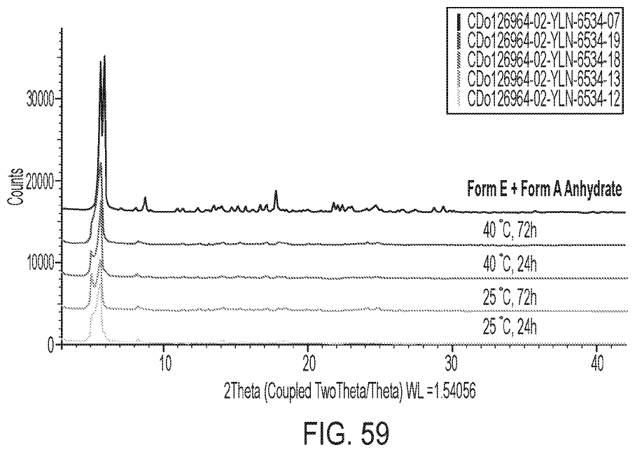

Experiment #3: Compound 1 Fumarate Form E (100 mg), Compound 1 Fumarate Form A Anhydrate (100 mg), and ethyl acetate (2 mL) were added into two 8-mL vials and mixed well in a shaker at 25° C. and 40° C., respectively. Samples for analysis were taken at 24 h and 72 h. Results of XRPD analysis are shown in FIG. 59. After 72 h at either 25° C. or 40° C., a mixture of Compound 1 Fumarate Form E and Compound 1 Fumarate Form A Anhydrate remained, indicating that conversion between the forms is slow in ethyl acetate.

### Example 12: Pharmacokinetic Studies in Beagle Dogs

Compound 1 (60 mg/mL) in two different forms was administered orally (PO) to male and female Beagle dogs. Blood was serially collected up to 144 hours post dose for determination of plasma pharmacokinetic analysis. The two forms of Compound 1 were Amorphous Compound 1 Free Base and Compound 1 Fumarate Form A Ethyl Acetate Solvate, each provided in a capsule.

Animals were healthy at the start of the study and were between 1 and 6.5 years of age. Initial body weights were recorded at the start of the study and general health observations were recorded at each blood collection time point.

Each Group contained 3 male and 1 female dog. Capsules were administered by placing the capsule to the back of the throat, followed by a 10 mL flush with drinking water.

All animals survived the duration of the study. There were no clinical signs observed in dogs during dosing. There were no clinical signs observed during general health observation over the course of the study.

Plasma concentrations of Compound 1 were determined by LC-MS/MS. Pharmacokinetic parameters were determined using Phoenix WinNonlin (v8.0) non-compartmental analyses.

Results of the study are summarized in Table 27. After PO administration, Compound 1 plasma concentration-time curves were well defined over a 144 hour period. Independent of form, initial systemic Compound 1 plasma concentrations were observed beginning between 5 and 30 minutes post dose and through 144 hours. Cₘₐₓ and AUCₗₐₛₜ were highest in dogs administered Compound 1 Fumarate Form A Ethyl Acetate Solvate.

TABLE 27

| | Group 1 | Group 2 |
|---|---|---|
| | Amorphous | Compound 1 Fumarate Form |
| Form | Compound 1 | A Ethyl Acetate Solvate |
| Dose (mg/kg) | 5.34 | 7.4 |
| Cₘₐₓ (ng/mL) | 343 | 503 |
| tₘₐₓ (h) | 3.25 | 3.5 |
| t_{1/2} (h) | 56.4 | 49.6 |
| MRTₗₐₛₜ (h) | 37.8 | 39.8 |
| AUCₗₐₛₜ (h · ng/mL) | 7460 | 14607 |
| AUC_{∞} (h · ng/mL) | 8955 | 17271 |
| AUCₗₐₛₜ/D | 1297 | 2001 |
| (h · kg · ng/mL/mg) | | |
| AUC_{∞}/D | 1527 | 2351 |
| (h · kg · ng/mL/mg) | | |

1. A crystalline solid form of Compound 1:

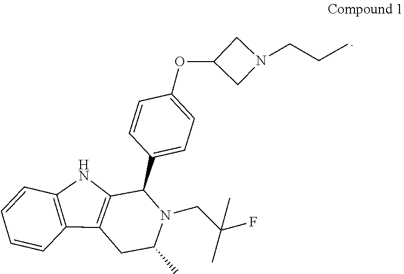 [SMILES: CCCN1CC(Oc2ccc([C@@H]3c4[nH]c5ccccc5c4C[C@@H](C)N3CC(C)(C)F)cc2)C1]

2. The solid form of claim 1, wherein the solid form is Form A.

3. The solid form of claim 2, wherein the solid form is an acetonitrile solvate (“Compound 1 Form A Acetonitrile Solvate”).

4. The solid form of claim 2 or 3, wherein the solid form is characterized by one or more peaks in its XRPD pattern selected from those at about 10.34, about 11.49, about 17.66, about 19.00, and about 19.76 degrees 2-theta.

5. The solid form of claim 2 or 3, wherein the solid is characterized by peaks in its XRPD pattern at about 10.34, about 11.49, about 17.66, about 19.00, and about 19.76 degrees 2-theta.

6. The solid form of claim 2 or 3, wherein the solid form is characterized by peaks in its XRPD pattern at substantially all of:
Position ± 0.2 (degrees 2-Theta)
5.31
10.34
10.61
11.49
12.40
13.50
14.72
16.04
17.45
17.66
18.02
18.43
19.00
19.76
20.44
20.66
21.60
22.03
23.03
23.87
25.07
25.27
26.37
27.10
27.65
29.13
30.63

7. The solid form of claim 3, wherein Compound 1 Form A Acetonitrile Solvate is characterized by one or more of the following:
(i) an XRPD pattern substantially similar to that depicted in FIG. 1 (top) and/or FIG. 6;
(ii) a TGA pattern substantially similar to that depicted in FIG. 2 and/or FIG. 7; and
(iii) a DSC pattern substantially similar to that depicted in FIG. 2 and/or FIG. 7.

8. The solid form of claim 2, wherein the solid form is an acetone solvate (“Compound 1 Form A Acetone Solvate”).

9. The solid form of claim 2 or 8, wherein the solid form is characterized by one or more peaks in its XRPD pattern selected from those at about 10.33, about 17.69, about 19.05, about 19.80, and about 20.49 degrees 2-theta.

10. The solid form of claim 2 or 8, wherein the solid is characterized by peaks in its XRPD pattern at about 10.33, about 17.69, about 19.05, about 19.80, and about 20.49 degrees 2-theta.

11. The solid form of claim 2 or 8, wherein the solid form is characterized by peaks in its XRPD pattern at substantially all of:
Position ± 0.2 (degrees 2-Theta)
4.72
5.31
10.33
11.51
13.54
14.72
17.47
17.69
18.47
19.05
19.80
20.49
21.69
23.94
25.13
27.22
29.21
33.71

12. The solid form of claim 8, wherein Compound 1 Form A Acetone Solvate is characterized by one or more of the following:
(i) an XRPD pattern substantially similar to that depicted in FIG. 1 (middle);
(ii) a TGA pattern substantially similar to that depicted in FIG. 9; and
(iii) a DSC pattern substantially similar to that depicted in FIG. 9.

13. The solid form of claim 2, wherein the solid form is a tetrahydrofuran solvate (“Compound 1 Form A Tetrahydrofuran Solvate”).

14. The solid form of claim 2 or 13, wherein the solid form is characterized by one or more peaks in its XRPD pattern selected from those at about 5.29, about 10.16, about 10.31, about 10.57, and about 19.75 degrees 2-theta.

15. The solid form of claim 2 or 13, wherein the solid is characterized by peaks in its XRPD pattern at about 5.29, about 10.16, about 10.31, about 10.57, and about 19.75 degrees 2-theta.

16. The solid form of claim 2 or 13, wherein the solid form is characterized by peaks in its XRPD pattern at substantially all of:
Position ± 0.2 (degrees 2-Theta)
5.29
10.16
10.31
10.57
11.47
13.52
14.69
15.86
17.81
18.42
18.99
19.75
20.40
20.73
21.57
22.31
23.04
23.83
23.95
25.04
27.12
27.72
29.12

17. The solid form of claim 13, wherein Compound 1 Form A Tetrahydrofuran Solvate is characterized by one or more of the following:
(i) an XRPD pattern substantially similar to that depicted in FIG. 1 (bottom);
(ii) a TGA pattern substantially similar to that depicted in FIG. 11; and
(iii) a DSC pattern substantially similar to that depicted in FIG. 11.

18. The solid form of claim 2, wherein the solid form is a dioxane solvate (“Compound 1 Form A Dioxane Solvate”).

19. The solid form of claim 2 or 18, wherein the solid form is characterized by one or more peaks in its XRPD pattern selected from those at about 10.10, about 10.25, about 17.54, about 18.82, about 19.64, and about 20.34 degrees 2-theta.

20. The solid form of claim 2 or 18, wherein the solid is characterized by peaks in its XRPD pattern at about 10.10, about 10.25, about 17.54, about 18.82, about 19.64, and about 20.34 degrees 2-theta.

21. The solid form of claim 2 or 18, wherein the solid form is characterized by peaks in its XRPD pattern at substantially all of:
Position ± 0.2 (degrees 2-Theta)
5.25
10.10
10.25
10.53
11.40
13.37
14.61
17.35
17.54
18.82
19.64
20.34
21.48
21.90

22. The solid form of claim 18, wherein Compound 1 Form A Dioxane Solvate is characterized by one or more of the following:
(i) an XRPD pattern substantially similar to that depicted in FIG. 13;
(ii) a TGA pattern substantially similar to that depicted in FIG. 14; and
(iii) a DSC pattern substantially similar to that depicted in FIG. 14.

23. The solid form of claim 2, wherein the solid form is an ethyl acetate solvate (“Compound 1 Form A Ethyl Acetate Solvate”).

24. The solid form of claim 2 or 23, wherein the solid form is characterized by one or more peaks in its XRPD pattern selected from those at about 10.10, about 10.25, about 17.54, about 18.82, about 19.64, and about 20.34 degrees 2-theta.

25. The solid form of claim 2 or 23, wherein the solid is characterized by peaks in its XRPD pattern at about 10.10, about 10.25, about 17.54, about 18.82, about 19.64, and about 20.34 degrees 2-theta.

26. The solid form of claim 2 or 23, wherein the solid form is characterized by peaks in its XRPD pattern at substantially all of:
Position ± 0.2 (degrees 2-Theta)
5.25
10.10
10.25
10.53
11.40
13.37
14.61
17.35
17.54
18.82
19.64
20.34
21.48
21.90

27. The solid form of claim 23, wherein Compound 1 Form A Ethyl Acetate Solvate is characterized by an XRPD pattern substantially similar to that depicted in FIG. 15.

28. The solid form of claim 2, wherein the solid form is a N-Methylpyrrolidone solvate (“Compound 1 Form A N-Methylpyrrolidone Solvate”).

29. The solid form of claim 28, wherein Compound 1 Form A N-Methylpyrrolidone Solvate is characterized by one or more of the following:
(i) an XRPD pattern substantially similar to that depicted in FIG. 12 (third from top);
(ii) a TGA pattern substantially similar to that depicted in FIG. 16; and
(iii) a DSC pattern substantially similar to that depicted in FIG. 16.

30. The solid form of claim 1, wherein the solid form is Form B.

31. The solid form of claim 30, wherein the solid form is characterized by one or more peaks in its XRPD pattern selected from those at about 8.85, about 11.73, about 14.19, about 17.71, and about 21.61 degrees 2-theta.

32. The solid form of claim 30, wherein the solid is characterized by peaks in its XRPD pattern at about 8.85, about 11.73, about 14.19, about 17.71, and about 21.61 degrees 2-theta.

33. The solid form of claim 30, wherein the solid form is characterized by peaks in its XRPD pattern at substantially all of:
Position ± 0.2 (degrees 2-Theta)
7.09
8.45
8.85
10.28
11.73
12.14
13.07
13.37
13.78
14.19
15.10
15.67
16.07
16.58
16.96
17.71
18.14
18.62
18.85
19.43
19.73
20.16
20.67
21.34
21.61
22.85
23.57
24.11
24.69
24.99
26.30
26.65
28.31

34. The solid form of claim 30, wherein Form B is characterized by one or more of the following:
(i) an XRPD pattern substantially similar to that depicted in FIG. 17;
(ii) a TGA pattern substantially similar to that depicted in FIG. 18; and
(iii) a DSC pattern substantially similar to that depicted in FIG. 18.

35. Compound 2: 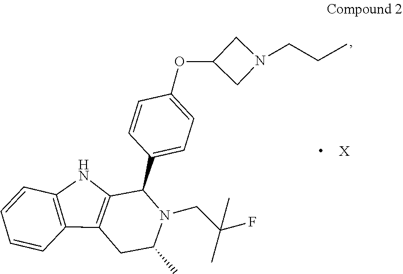
wherein X is a co-former selected from the group consisting of malic acid, fumaric acid, oxalic acid, and phosphoric acid.

36. A crystalline solid form of Compound 2.

37. The solid form of claim 36, wherein X is malic acid.

38. The solid form of claim 37, wherein the solid form is Compound 1 Malate Form A.

39. The solid form of claim 38, wherein the solid form is characterized by one or more peaks in its XRPD pattern selected from those at about 4.50, about 9.00, about 13.50, about 14.23, and about 18.59 degrees 2-theta.

40. The solid form of claim 38, wherein the solid is characterized by peaks in its XRPD pattern at about 4.50, about 9.00, about 13.50, about 14.23, and about 18.59 degrees 2-theta.

41. The solid form of claim 38, wherein the solid form is characterized by peaks in its XRPD pattern at substantially all of:
Position ± 0.2 (degrees 2-Theta)
4.50
9.00
10.06
12.53
12.73
13.50
14.23
17.91
18.59
19.53
20.18
21.06
22.59
23.06
24.36
27.56

42. The solid form of claim 38, wherein Compound 1 Malate Form A is characterized by one or more of the following:
(i) an XRPD pattern substantially similar to that depicted in FIG. 21;
(ii) a TGA pattern substantially similar to that depicted in FIG. 22; and
(iii) a DSC pattern substantially similar to that depicted in FIG. 22.

43. The solid form of claim 36, wherein X is fumaric acid.

44. The solid form of claim 43, wherein the solid form is Compound 1 Fumarate Form A Anhydrate.

45. The solid form of claim 44, wherein the solid form is characterized by one or more peaks in its XRPD pattern selected from those at about 5.77, about 8.23, about 9.25, about 11.47, about 12.50, about 15.28, and about 17.23 degrees 2-theta.

46. The solid form of claim 44, wherein the solid is characterized by peaks in its XRPD pattern at about 5.77, about 8.23, about 9.25, about 11.47, about 12.50, about 15.28, and about 17.23 degrees 2-theta.

47. The solid form of claim 44, wherein Compound 1 Fumarate Form A Anhydrate is characterized by peaks in its XRPD pattern at substantially all of:
Position ± 0.2 (degrees 2-Theta)
5.77
8.23
9.25
11.47
12.50
13.20
13.54
13.95
14.16
15.28
16.39
17.23
17.95
18.40
19.24
19.53
19.98
20.39
21.04
22.58
23.18
24.19
24.76
25.15
25.77
26.47
27.91
28.90
29.51
34.92
38.93

48. The solid form of claim 44, wherein Compound 1 Fumarate Form A Anhydrate is characterized by one or more of the following:
(i) an XRPD pattern substantially similar to that depicted in FIG. 24 and/or FIG. 42;
(ii) a TGA pattern substantially similar to that depicted in FIG. 25 and/or FIG. 43; and
(iii) a DSC pattern substantially similar to that depicted in FIG. 25 and/or FIG. 43.

49. The solid form of claim 43, wherein the solid form is Compound 1 Fumarate Form A Ethyl Acetate Solvate.

50. The solid form of claim 49, wherein the solid form is characterized by one or more peaks in its XRPD pattern selected from those at about 4.94, about 8.18, about 14.14, about 15.95, about 16.38, and about 18.54 degrees 2-theta.

51. The solid form of claim 49, wherein the solid is characterized by peaks in its XRPD pattern at about 4.94, about 8.18, about 14.14, about 15.95, about 16.38, and about 18.54 degrees 2-theta.

52. The solid form of claim 49, wherein the solid form is characterized by peaks in its XRPD pattern at substantially all of:
Position ± 0.2 (degrees 2-Theta)
4.94
8.18
11.85
12.54
14.14
14.68
14.96
15.95
16.38
17.25
18.12
18.54
19.86
20.71

53. The solid form of claim 49, wherein Compound 1 Fumarate Form A Ethyl Acetate Anhydrate is characterized by one or more of the following:
(i) an XRPD pattern substantially similar to that depicted in FIG. 27 and/or FIG. 28;
(ii) a TGA pattern substantially similar to that depicted in FIG. 29; and
(iii) a DSC pattern substantially similar to that depicted in FIG. 29.

54. The solid form of claim 43, wherein the solid form is Compound 1 Fumarate Form C.

55. The solid form of claim 54, wherein the solid form is characterized by one or more peaks in its XRPD pattern selected from those at about 5.67, about 8.14, about 12.40, about 13.84, about 14.04, and about 15.16 degrees 2-theta.

56. The solid form of claim 54, wherein the solid is characterized by peaks in its XRPD pattern at about 5.67, about 8.14, about 12.40, about 13.84, about 14.04, and about 15.16 degrees 2-theta.

57. The solid form of claim 54, wherein the solid form is characterized by peaks in its XRPD pattern at substantially all of:
Position ± 0.2 (degrees 2-Theta)
5.67
6.52
8.14
9.14
11.38
11.95
12.4
13.07
13.44
13.84
14.04
15.16
17.82
19.45

58. The solid form of claim 54, wherein Compound 1 Fumarate Form C is characterized by an XRPD pattern substantially similar to that depicted in FIG. 31 or those depicted in FIG. 41 (top three).

59. The solid form of claim 43, wherein the solid form is Compound 1 Fumarate Form D.

60. The solid form of claim 59, wherein the solid form is characterized by one or more peaks in its XRPD pattern selected from those at about 4.84, about 8.08, about 14.08, about 15.73, and about 18.40 degrees 2-theta.

61. The solid form of claim 59, wherein the solid is characterized by peaks in its XRPD pattern at about 4.84, about 8.08, about 14.08, about 15.73, and about 18.40 degrees 2-theta.

62. The solid form of claim 59, wherein the solid form is characterized by peaks in its XRPD pattern at substantially all of:
Position ± 0.2 (degrees 2-Theta)
4.84
8.08
12.49
13.46
14.08
14.09
14.11
14.54
14.83
15.73
16.18
17.02
18.40
20.41

63. The solid form of claim 59, wherein Compound 1 Fumarate Form D is characterized by one or more of the following:
(i) an XRPD pattern substantially similar to that depicted in FIG. 33;
(ii) a TGA pattern substantially similar to that depicted in FIG. 34; and
(iii) a DSC pattern substantially similar to that depicted in FIG. 34.

64. The solid form of claim 43, wherein the solid form is Compound 1 Fumarate Form F.

65. The solid form of claim 64, wherein the solid form is characterized by one or more peaks in its XRPD pattern selected from those at about 4.77, about 8.03 about 14.56, about 15.90, about 17.82, and about 24.77 degrees 2-theta.

66. The solid form of claim 64, wherein the solid is characterized by peaks in its XRPD pattern at out 4.77, about 8.03 about 14.56, about 15.90, about 17.82, and about 24.77 degrees 2-theta.

67. The solid form of claim 64, wherein the solid form is characterized by peaks in its XRPD pattern at substantially all of:
Position ± 0.2 (degrees 2-Theta)
4.77
8.03
11.54
12.91
13.49
13.98
14.56
15.90
17.17
17.82
20.53
23.10
23.52
24.77

68. The solid form of claim 64, wherein Compound 1 Fumarate Form F is characterized by one or more of the following:
(i) an XRPD pattern substantially similar to that depicted in FIG. 37;
(ii) a TGA pattern substantially similar to that depicted in FIG. 38; and
(iii) a DSC pattern substantially similar to that depicted in FIG. 38.

69. The solid form of claim 43, wherein the solid form is Compound 1 Fumarate Form G.

70. The solid form of claim 69, wherein the solid form is characterized by one or more peaks in its XRPD pattern selected from those at about 5.49, about 8.43, about 12.74, about 13.69, and about 19.56 degrees 2-theta.

71. The solid form of claim 69, wherein the solid is characterized by peaks in its XRPD pattern at about 5.49, about 8.43, about 12.74, about 13.69, and about 19.56 degrees 2-theta.

72. The solid form of claim 69, wherein the solid form is characterized by peaks in its XRPD pattern at substantially all of:
Position ± 0.2 (degrees 2-Theta)
5.49
8.43
12.10
12.74
13.69
14.23
14.71
17.67
19.56
20.98

73. The solid form of claim 69, wherein Compound 1 Fumarate Form G is characterized by one or more of the following:
(i) an XRPD pattern substantially similar to that depicted in FIG. 39;
(ii) a TGA pattern substantially similar to that depicted in FIG. 40; and
(iii) a DSC pattern substantially similar to that depicted in FIG. 40.

74. The solid form of claim 43, wherein the solid form is Compound 1 Fumarate Form J.

75. The solid form of claim 74, wherein the solid form is characterized by one or more peaks in its XRPD pattern selected from those at about 8.28, about 9.18, about 9.53, about 9.77, about 18.66, and about 19.19 degrees 2-theta.

76. The solid form of claim 74, wherein the solid is characterized by peaks in its XRPD pattern at about 8.28, about 9.18, about 9.53, about 9.77, about 18.66, and about 19.19 degrees 2-theta.

77. The solid form of claim 74, wherein the solid form is characterized by peaks in its XRPD pattern at substantially all of:
Position ± 0.2 (degrees 2-Theta)
4.37
5.34
8.28
9.18
9.53
9.77
11.79
13.81
17.03
18.66
19.19

78. The solid form of claim 74, wherein Compound 1 Fumarate Form J is characterized by an XRPD pattern substantially similar to that depicted in FIG. 41 (fourth from the top).

79. The solid form of claim 36, wherein X is oxalic acid.

80. The solid form of claim 79, wherein the solid form is Compound 1 Oxalate Form A.

81. The solid form of claim 80, wherein the solid form is characterized by one or more peaks in its XRPD pattern selected from those at about 4.68, about 8.95, about 9.37, about 12.76, about 14.07, about 18.78 degrees 2-theta.

82. The solid form of claim 80, wherein the solid is characterized by peaks in its XRPD pattern at about 4.68, about 8.95, about 9.37, about 12.76, about 14.07, about 18.78 degrees 2-theta.

83. The solid form of claim 80, wherein the solid form is characterized by peaks in its XRPD pattern at substantially all of:
Position ± 0.2 (degrees 2-Theta)
4.68
8.95
9.37
9.83
11.14
11.58
12.76
14.07
14.47
16.54
18.78
19.67
20.92
21.45
22.90
23.55
28.32

84. The solid form of claim 80, wherein Compound 1 Oxalate Form A is characterized by one or more of the following:
(i) an XRPD pattern substantially similar to that depicted in FIG. 45;
(ii) a TGA pattern substantially similar to that depicted in FIG. 46; and
(iii) a DSC pattern substantially similar to that depicted in FIG. 46.

85. The solid form of claim 36, wherein X is phosphoric acid.

86. The solid form of claim 85, wherein the solid form is Compound 1 Phosphate Form A.

87. The solid form of claim 86, wherein the solid form is characterized by one or more peaks in its XRPD pattern selected from those at about 3.86, about 4.30, about 5.05, about 13.76, about 17.85, and about 20.42 degrees 2-theta.

88. The solid form of claim 86, wherein the solid is characterized by peaks in its XRPD pattern at about 3.86, about 4.30, about 5.05, about 13.76, about 17.85, and about 20.42 degrees 2-theta.

89. The solid form of claim 86, wherein the solid form is characterized by peaks in its XRPD pattern at substantially all of:
Position ± 0.2 (degrees 2-Theta)
3.86
4.30
5.05
7.72
8.57
10.01
12.84
13.76
14.82
15.57
16.93
17.20
17.85
18.71
19.17
19.82
20.42
21.04
21.49
21.85
22.17
22.76
23.89
24.24
24.77
25.34
25.81
26.35
30.26
32.75

90. The solid form of claim 86, wherein Compound 1 Phosphate Form A is characterized by one or more of the following:
(i) an XRPD pattern substantially similar to that depicted in FIG. 47;
(ii) a TGA pattern substantially similar to that depicted in FIG. 48; and
(iii) a DSC pattern substantially similar to that depicted in FIG. 48.

91. The solid form of claim 85, wherein the solid form is Compound 1 Phosphate Form B.

92. The solid form of claim 91, wherein the solid form is characterized by one or more peaks in its XRPD pattern selected from those at about 4.38, about 6.54, about 13.07, about 14.89, and about 23.76 degrees 2-theta.

93. The solid form of claim 91, wherein the solid is characterized by peaks in its XRPD pattern at about 4.38, about 6.54, about 13.07, about 14.89, and about 23.76 degrees 2-theta.

94. The solid form of claim 91, wherein the solid form is characterized by peaks in its XRPD pattern at substantially all of:
ion ± 0.2 (degrees 2-Theta
3.29
4.38
6.54
7.72
8.70
9.04
10.93
13.07
14.89
16.94
17.47
19.70
21.18
23.76
24.10
25.08

95. The solid form of claim 91, wherein Compound 1 Phosphate Form B is characterized by one or more of the following:
(i) an XRPD pattern substantially similar to that depicted in FIG. 49;
(ii) a TGA pattern substantially similar to that depicted in FIG. 50; and
(iii) a DSC pattern substantially similar to that depicted in FIG. 50.

96. A solid form obtainable by a process described herein (e.g., in Example 2 or Example 3).

97. The solid form of claim 96, wherein the solid form is the solid form of any one of claims 1-34 and 36-95.

98. A pharmaceutical composition comprising the compound of claim 35 or the solid form of any one of claims 1-34 and 36-97 and a pharmaceutically acceptable carrier.

99. The pharmaceutical composition of claim 98, wherein the pharmaceutical composition is solid.

100. The pharmaceutical composition of claim 98 or 99, wherein the pharmaceutical composition is formulated for oral administration.

101. A method of inhibiting an estrogen receptor or mutant thereof in a biological sample comprising contacting the biological sample with the compound of claim 35 or the solid form of any one of claims 1-34 and 36-97.

102. A method of inhibiting an estrogen receptor or mutant thereof in a patient comprising contacting the patient with the compound of claim 35 or the solid form of any one of claims 1-34 and 36-97.

103. A method of treating a disease, disorder, or condition associated with the estrogen receptor in a patient, comprising administering to the patient a therapeutically effective amount of the compound of claim 35 or the solid form of any one of claims 1-34 and 36-97.

104. The method of claim 103, wherein the disease, disorder, or condition is selected from the group consisting of breast cancer, bone cancer, lung cancer, colorectal cancer, endometrial cancer, prostate cancer, ovarian cancer, vaginal cancer, endometriosis, and uterine cancer.

105. The method of claim 103, wherein the disease, disorder, or condition is breast cancer.

106. The method of any one of claims 103-105, further comprising administering another anti-cancer agent.

107. The method of claim 106, wherein the anti-cancer agent is a CDK4/6 inhibitor, a PI3KCA inhibitor, or an mTOR inhibitor.

108. A method of preparing the compound of claim 35 or the solid form of any one of claims 1-34 and 36-97 according to a method described herein.
